(19) United States
(12) Patent Application Publication
(10) Pub. No.: US 2025/0376726 A1
(43) Pub. Date: Dec. 11, 2025

(54) METHODS AND COMPOSITIONS FOR IN SITU ANALYSIS OF DNA METHYLATION

(71) Applicant: 10x Genomics, Inc., Pleasanton, CA (US)

(72) Inventor: Robert HENLEY, Castro Valley, CA (US)

(21) Appl. No.: 19/231,198

(22) Filed: Jun. 6, 2025

Related U.S. Application Data
(60) Provisional application No. 63/657,708, filed on Jun. 7, 2024.

(51) Int. Cl. C12Q 1/6874; C12Q 1/6886

(52) U.S. Cl. CPC C12Q 1/6874; C12Q 1/6886; C12Q 2600/154

(57) ABSTRACT

The present disclosure generally relates to methods and compositions for interrogating and/or analyzing DNA methylation in a biological sample. In some aspects, the present disclosure relates to methods for determining the methylation status of a region of interest of genomic DNA. In some aspects, the methylation status is analyzed by interrogating converted DNA in which the sequence of the converted DNA is indicative of the methylation state of the DNA. In some aspects, the methods comprise generating a collective signal that is based on the methylation states of a plurality of sequences or residues in the DNA, and that is representative of the methylation status of the region of interest as a whole.

### CROSS-REFERENCE TO RELATED APPLICATIONS

This application claims priority to U.S. Provisional Patent Application No. 63/657,708, filed Jun. 7, 2024, entitled “METHODS AND COMPOSITIONS FOR IN SITU ANALYSIS OF DNA METHYLATION,” which is herein incorporated by reference in its entirety for all purposes.

### FIELD

The present disclosure generally relates to methods and compositions for interrogating and/or analyzing DNA methylation.

### BACKGROUND

DNA methylation analysis can provide valuable insight into gene regulation and identify potential biomarkers. Aberrant DNA methylation has been implicated in many disease processes, including cancer, obesity, and addiction. Given the value of potential insights based on DNA methylation analysis, there is a need for improved methods of DNA methylation analysis.

### SUMMARY

In some aspects, provided herein are methods for interrogating and/or analyzing methylation, such as interrogating and/or analyzing a methylation status of a region of interest in a deoxyribonucleic acid (DNA). In some aspects, the methods comprise providing a biological sample comprising converted DNA generated by converting the DNA. In some aspects, the nucleotide sequence of the converted DNA is indicative of the methylation state of the DNA. In some aspects, the methods comprise interrogating the converted DNA to analyze methylation of the DNA (e.g. the methylation status of the DNA). In some aspects, the methods comprise generating a collective signal based on the methylation states of a plurality of sequences or residues in the DNA that is representative of the methylation status of the region of interest as a whole. In some aspects, the methods can facilitate analysis of methylation at one or more regions of interest. In some aspects, the methods involve interrogation and/or analysis of methylation in situ in a biological sample.

In some aspects, provided herein is a method of analyzing a methylation status of a region of interest in a deoxyribonucleic acid (DNA) comprising: providing a biological sample comprising converted DNA generated by converting the DNA, wherein the nucleotide sequence of the converted DNA is indicative of the methylation state of the DNA; contacting the biological sample with a plurality of methylation-state-specific probes, wherein each methylation-state-specific probe is complementary to a converted DNA target sequence indicative of a methylation state of a target sequence in the region of interest, and wherein the plurality of methylation state-specific probes collectively target a plurality of converted DNA target sequences corresponding to a plurality of target sequences in the region of interest; detecting a methylation-state-specific signal associated with hybridization of methylation-state-specific probes to the converted DNA; and using the methylation-state-specific signal to analyze the methylation status of the region of interest. In some aspects, provided herein is a method for interrogating methylation of a region of interest in a deoxyribonucleic acid (DNA) comprising: providing a biological sample comprising converted DNA generated by converting the DNA, wherein the nucleotide sequence of the converted DNA is indicative of the methylation state of the DNA; contacting the biological sample with a plurality of methylation-state-specific probes, wherein each methylation-state-specific probe is complementary to a converted DNA target sequence indicative of a methylation state of a target sequence in the region of interest, and wherein the plurality of methylation state-specific probes collectively target a plurality of converted DNA target sequences corresponding to a plurality of target sequences in the region of interest; and detecting a methylation-state-specific signal associated with hybridization of methylation-state-specific probes to the converted DNA. In some embodiments, the method comprises using the methylation-state-specific signal to analyze the methylation status of the region of interest.

In some embodiments, the methylation-state-specific signal is collectively generated from the methylation-state-specific probes that hybridize to the converted DNA.

In some embodiments, the plurality of methylation-state-specific probes is a plurality of first methylation-state-specific probes; each first methylation-state-specific probe is complementary to a converted DNA target sequence indicative of a first methylation state of a target sequence in the region of interest; the methylation-state-specific signal is a first methylation-state-specific signal; and the method comprises detecting the first methylation-state-specific signal associated with hybridization of first methylation-state-specific probes to the converted DNA. In some embodiments, the method comprises using the first methylation-state-specific signal to analyze the methylation status of the region of interest.

In some embodiments, the method further comprises: contacting the biological sample with a plurality of second methylation-state-specific probes, wherein each second methylation-state-specific probe is complementary to a converted DNA target sequence indicative of a second methylation state of a target sequence in the region of interest, wherein the plurality of second methylation state-specific probes collectively target a plurality of converted DNA target sequences corresponding to a plurality of target sequences in the region of interest; and detecting a second methylation-state-specific signal associated with hybridization of second methylation-state-specific probes to the converted DNA. In some embodiments, the method comprises using the second methylation-state-specific signal to analyze the methylation status of the region of interest.

In some embodiments, the first methylation-state-specific signal is collectively generated from the first methylation-state-specific probes that hybridize to the converted DNA; and/or the second methylation-state-specific signal is collectively generated from the second methylation-state-specific probes that hybridize to the converted DNA.

In some embodiments, the plurality of converted DNA target sequences targeted by the first methylation state-specific probes and the plurality of converted DNA target sequences targeted by the second methylation state-specific probes correspond to the same plurality of target sequences in the region of interest. In some embodiments, the plurality of converted DNA target sequences targeted by the first methylation state-specific probes and the plurality of converted DNA target sequences targeted by the second methylation state-specific probes do not correspond to the same plurality of target sequences in the region of interest.

In some embodiments, the region of interest is at least 200 bases, at least 500 bases, or at least 1000 bases in length.

In some embodiments, the plurality of methylation-state-specific probes comprises at least 3, 5, 10, 20, 50, 100, or 500 methylation-state-specific probes; the plurality of first methylation-state-specific probes comprises at least 3, 5, 10, 20, 50, 100, or 500 first methylation-state-specific probes; and/or the plurality of second methylation-state-specific probes comprises at least 3, 5, 10, 20, 50, 100, or 500 second methylation-state-specific probes.

In some embodiments, each target sequence in the region of interest comprises 1, 2, 3, 4, or more cytosine residues. In some embodiments, each target sequence in the region of interest comprises 1, 2, 3, 4, or more CpG cytosine residues. In some embodiments, each target sequence in the region of interest is independently between 10 and 50 nucleotides in length.

In some embodiments, analyzing the methylation status of the region of interest comprises measuring the size, intensity, and/or abundance of the methylation-state-specific signal, the first methylation-state-specific signal, the second methylation-state-specific signal, and/or a reference signal. In some embodiments, the method comprises measuring the size, intensity, and/or abundance of the methylation-state-specific signal, the first methylation-state-specific signal, the second methylation-state-specific signal, and/or a reference signal. In some embodiments, analyzing the methylation status of the region of interest in the DNA comprises comparing the methylation-state-specific signal, the first methylation-state-specific signal, and/or the second methylation-state-specific signal to the reference signal. In some embodiments, the method comprises comparing the methylation-state-specific signal, the first methylation-state-specific signal, and/or the second methylation-state-specific signal to the reference signal. In some embodiments, increasing size, intensity, and/or abundance of the first methylation-state-specific signal is indicative of a methylation status of the region of interest that is increasingly similar to the first methylation states of the target sequences in the region of interest. In some embodiments, increasing size, intensity, and/or abundance of the first methylation-state-specific signal in comparison to the reference signal is indicative of a methylation status of the region of interest that is increasingly similar to the first methylation states of the target sequences in the region of interest.

In some embodiments, analyzing the methylation status of the region of interest in the DNA comprises comparing the first methylation-state-specific signal to the second methylation-state-specific signal. In some embodiments, the method comprises comparing the first methylation-state-specific signal to the second methylation-state-specific signal. In some embodiments, increasing size, intensity, and/or abundance of the first methylation-state-specific signal in comparison to the second methylation-state-specific signal is indicative of a methylation status of the region of interest that is increasingly similar to the first methylation states of the target sequences in the region of interest than to the second methylation states of the target sequences in the region of interest.

In some embodiments, the first methylation states of the target sequences in the region of interest comprise fewer methylated cytosines than the second methylation states of the target sequences in the region of interest; the first methylation states of the target sequences in the region of interest comprise a smaller proportion of methylated CpG cytosine residues than the second methylation states of the target sequences in the region of interest; the first methylation states are methylation states in which none of the CpG cytosine residues in the target sequences in the region of interest are methylated; and/or the second methylation states are methylation states in which all of the CpG cytosine residues in the target sequences in the region of interest are methylated. In some embodiments, increased size, intensity, and/or abundance of the first methylation-state-specific signal in comparison to a reference signal or to the second methylation-state-specific signal is indicative of a methylation status of the region of interest in which a lower proportion of cytosine residues are methylated.

In some embodiments, the first methylation states of the target sequences in the region of interest comprise more methylated cytosines than the second methylation states of the target sequences in the region of interest; the first methylation states of the target sequences in the region of interest comprise a larger proportion of methylated CpG cytosine residues than the second methylation states of the target sequences in the region of interest; the first methylation states are methylation states in which all of the CpG cytosine residues in the target sequences in the region of interest are methylated; and/or the second methylation states are methylation states in which none of the CpG cytosine residues in the target sequences in the region of interest are methylated.

In some embodiments, increased size, intensity, and/or abundance of the first methylation-state-specific signal in comparison to a reference signal or to the second methylation-state-specific signal is indicative of a methylation status of the region of interest in which a higher proportion of cytosine residues are methylated.

In some embodiments, the methylation-state-specific probes are directly or indirectly associated with a detectable label, and detecting the methylation-state-specific signal comprises detecting the detectable label; the first methylation-state-specific probes are directly or indirectly associated with a first detectable label, and detecting the first methylation-state-specific signal comprises detecting the first detectable label; and/or the second methylation-state-specific probes are directly or indirectly associated with a second detectable label, and detecting the second methylation-state-specific signal comprises detecting the second detectable label.

In some embodiments, the methylation-state-specific probes are directly associated with the detectable label, the first methylation-state-specific probes are directly associated with the first detectable label, and/or the second methylation-state-specific probes are directly associated with the second detectable label.

In some embodiments, the methylation-state-specific probes are configured to directly or indirectly bind and/or hybridize to detectably labeled probes comprising the detectable label, the first methylation-state-specific probes are configured to directly or indirectly bind and/or hybridize to detectably labeled probes comprising the first detectable label, and/or the second methylation-state-specific probes are configured to directly or indirectly bind and/or hybridize to detectably labeled probes comprising the second detectable label. In some embodiments, the detectable label, the first detectable label, and/or the second detectable label are fluorophores.

In some embodiments, one or more of the methylation-state-specific probes, first methylation-state-specific probes, and/or second methylation-state-specific probes are independently selected from the group consisting of: a probe not comprising an overhang, a probe comprising a 3′ and/or 5′ overhang; a circular probe; and a circularizable probe or probe set.

In some embodiments, one or more of the methylation-state-specific probes, first methylation-state-specific probes, and/or second methylation-state-specific probes comprise a barcode region associated with: a) the region of interest, b) the target sequence in the region of interest corresponding to the converted DNA target sequence to which it hybridizes, and/or c) the methylation state of the target sequence in the region of interest corresponding to the converted DNA target sequence to which it hybridizes.

In some embodiments, the methylation-state-specific signal, the first methylation-state-specific signal, and/or the second methylation-state-specific signal is amplified. In some embodiments, the signal amplification comprises using the methylation-state-specific probes, first methylation-state-specific probes, and/or second methylation-state-specific probes to perform: rolling circle amplification (RCA); hybridization chain reaction (HCR); linear oligonucleotide hybridization chain reaction (LO-HCR); primer exchange reaction (PER); assembly of branched structures; hybridization of a plurality of detectable probes directly or indirectly on the methylation-state-specific probes, first methylation-state-specific probes, and/or second methylation-state-specific probes or products thereof; or any combination thereof.

In some aspects, provided herein is a method of analyzing a methylation status of a region of interest in a deoxyribonucleic acid (DNA) comprising: providing a biological sample comprising converted DNA generated by converting the DNA, wherein the nucleotide sequence of the converted DNA is indicative of the methylation state of the DNA, contacting the biological sample with a plurality of competing probe sets, each competing probe set comprising: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest; detecting a first signal associated with hybridization of first competing probes of the plurality of competing probe sets to the converted DNA; and using the first signal to analyze the methylation status of the region of interest in the DNA.

In some aspects, provided herein is a method for interrogating methylation of a region of interest in a deoxyribonucleic acid (DNA) comprising: providing a biological sample comprising converted DNA generated by converting the DNA, wherein the nucleotide sequence of the converted DNA is indicative of the methylation state of the DNA, contacting the biological sample with a plurality of competing probe sets, each competing probe set comprising: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest; and detecting a first signal associated with hybridization of first competing probes of the plurality of competing probe sets to the converted DNA.

In some embodiments, the method further comprises detecting a second signal associated with hybridization of second competing probes of the plurality of competing probe sets to the converted DNA. In some embodiments, the method comprises using the first signal and/or the second signal to analyze the methylation status of the region of interest in the DNA. In some embodiments, the method comprises using the first signal and the second signal to analyze the methylation status of the region of interest in the DNA.

In some embodiments, one or more competing probe sets of the plurality of competing probe sets further comprise a third competing probe that is complementary to a third converted DNA target sequence indicative of a third methylation state of the target sequence in the region of interest. In some embodiments, the method comprises detecting a third signal associated with hybridization of third competing probes of the plurality of competing probe sets to the converted DNA. In some embodiments, the method further comprises using the first signal, second signal, and third signal to analyze the methylation status of the region of interest in the DNA.

In some embodiments, one or more competing probe sets of the plurality of competing probe sets comprise further competing probes that are complementary to further converted DNA target sequences indicative of further methylation states of the target sequence in the region of interest. In some embodiments, the method further comprises detecting further signals associated with hybridization of further competing probes of the plurality of competing probe sets to the converted DNA. In some embodiments, the method comprises using the first signal, second signal, third signal, and/or further signals to analyze the methylation status of the region of interest in the DNA.

In some embodiments, the first signal corresponds to the first methylation states of the target sequences; the second signal corresponds to the second methylation states of the target sequences; the third signal corresponds to the third methylation states of the target sequences; and/or the further signals correspond to further methylation states of the target sequences.

In some embodiments, the plurality of competing probe sets comprises a first competing probe set comprising competing probes complementary to converted DNA target sequences indicative of different methylation states of a first target sequence in the region of interest; and a second competing probe set comprising competing probes complementary to converted DNA target sequences indicative of different methylation states of a second target sequence in the region of interest. In some embodiments, the plurality of competing probe sets further comprises a third competing probe set comprising competing probes complementary to converted DNA target sequences indicative of different methylation states of a third target sequence in the region of interest. In some embodiments, the plurality of competing probe sets comprises further competing probe sets, each further competing probe set comprising competing probes complementary to converted DNA target sequences indicative of different methylation states of further target sequences in the region of interest.

In some embodiments, the plurality of competing probe sets comprises at least 10, 20, 50, 100, or 500 competing probe sets for at least 10, 20, 50, 100, or 500 target sequences in the region of interest, respectively. In some embodiments, the plurality of competing probe sets comprises at least 3, 5, 10, 20, 50, 100, or 500 competing probe sets for at least 3, 5, 10, 20, 50, 100, or 500 target sequences in the region of interest, respectively. In some embodiments, a competing probe set is provided for each of the target sequences in the region of interest.

In some embodiments, the first, second, third, and/or further methylation states of the target sequences in the region of interest each comprise a different proportion of methylated CpG cytosine residues. In some embodiments, each target sequence in the region of interest comprises 1, 2, 3, 4, or more cytosine residues. In some embodiments, each target sequence in the region of interest comprises 1, 2, 3, 4, or more CpG cytosine residues. In some embodiments, each target sequence in the region of interest is independently between 10 and 50 nucleotides in length.

In some embodiments, the method comprises measuring the size, intensity, and/or abundance of the first signal, second signal, third signal, further signals, and/or a reference signal. In some embodiments, the method comprises comparing at least two of: the first signal, second signal, third signal, further signals, and reference signal. In some embodiments, the method comprises comparing the first signal, second signal, third signal, and/or further signals to the reference signal. In some embodiments, analyzing the methylation status of the region of interest comprises measuring the size, intensity, and/or abundance of the first signal, second signal, third signal, further signals, and/or a reference signal. In some embodiments, analyzing the methylation status of the region of interest comprises comparing at least two of: the first signal, second signal, third signal, further signals, and reference signal. In some embodiments, analyzing the methylation status of the region of interest comprises comparing the first signal, second signal, third signal, and/or further signals to the reference signal.

In some embodiments, increasing size, intensity, and/or abundance of a detected signal is indicative of a methylation status of the region of interest that is increasingly similar to the methylation state to which the signal corresponds. In some embodiments, increasing size, intensity, and/or abundance of the first signal is indicative of a methylation status of the region of interest that is increasingly similar to the first methylation states of the target sequences in the region of interest. In some embodiments, increasing size, intensity, and/or abundance of the first signal in comparison to the reference signal is indicative of a methylation status of the region of interest that increasingly similar to the first methylation states of the target sequences in the region of interest. In some embodiments, the method comprises comparing the first signal to the second signal. In some embodiments, analyzing the methylation status of the region of interest comprises comparing the first signal to the second signal.

In some embodiments, increasing size, intensity, and/or abundance of the first signal in comparison to the second signal is indicative of a methylation status of the region of interest that is increasingly similar to the first methylation states of the target sequences in the region of interest than to the second methylation states of the target sequences in the region of interest.

In some embodiments, the first methylation states of the target sequences in the region of interest comprise fewer methylated cytosines than the second methylation states of the target sequences in the region of interest; the first methylation states of the target sequences in the region of interest comprise a smaller proportion of methylated CpG cytosine residues than the second methylation states of the target sequences in the region of interest; the first methylation states are methylation states in which none of the CpG cytosine residues in the target sequences in the region of interest are methylated; and/or the second methylation states are methylation states in which all of the CpG cytosine residues in the target sequences in the region of interest are methylated. In some embodiments, increased size, intensity, and/or abundance of the first signal in comparison to the second signal or to the reference signal is indicative of a methylation status of the region of interest in which a lower proportion of cytosine residues are methylated.

In some embodiments, the first methylation states of the target sequences in the region of interest comprise more methylated cytosines than the second methylation states of the target sequences in the region of interest; the first methylation states of the target sequences in the region of interest comprise a larger proportion of methylated CpG cytosine residues than the second methylation states of the target sequences in the region of interest; the first methylation states are methylation states in which all of the CpG cytosine residues in the target sequences in the region of interest are methylated; and/or the second methylation states are methylation states in which none of the CpG cytosine residues in the target sequences in the region of interest are methylated. In some embodiments, increased size, intensity, and/or abundance of the first signal in comparison to the second signal or to the reference signal is indicative of a methylation status of the region of interest in which a higher proportion of cytosine residues are methylated.

In some embodiments, the first competing probes of the plurality of competing probe sets are directly or indirectly associated with a first detectable label corresponding to the first methylation state, and detecting the first signal comprises detecting the first detectable label; the second competing probes of the plurality of competing probe sets are directly or indirectly associated with a second detectable label corresponding to the second methylation state, and detecting the second signal comprises detecting the second detectable label; and/or the third and/or further competing probes of the plurality of competing probe sets are directly or indirectly associated with third and/or further detectable labels corresponding to third and/or further methylation states, and detecting the third and/or further signals comprises detecting the third and/or further detectable labels.

In some embodiments, one or more competing probes of the plurality of competing probe sets are directly associated with the detectable labels. In some embodiments, one or more competing probes of the plurality of competing probe sets are configured to directly or indirectly bind and/or hybridize to detectably labeled probes comprising the detectable labels. In some embodiments, the detectable labels are fluorophores.

In some embodiments, one or more competing probes of the plurality of competing probe sets are independently selected from the group consisting of: a probe not comprising an overhang, a probe comprising a 3′ and/or 5′ overhang; a circular probe; and a circularizable probe or probe set.

In some embodiments, one or more competing probes of the plurality of competing probe sets comprises a barcode region associated with: a) the region of interest, b) the target sequence in the region of interest corresponding to the converted DNA target sequence to which it hybridizes, and/or c) the converted DNA target sequence to which it hybridizes.

In some embodiments, the first signal, second signal, third signal, and/or further signals, are amplified. In some embodiments, the signal amplification comprises using one or more of the competing probes of the plurality of competing probe sets to perform: rolling circle amplification (RCA); hybridization chain reaction (HCR); linear oligonucleotide hybridization chain reaction (LO-HCR); primer exchange reaction (PER); assembly of branched structures; hybridization of a plurality of detectable probes directly or indirectly on the competing probes or products thereof; or any combination thereof.

In some embodiments, the first signal is collectively generated from the first competing probes of the plurality of competing probe sets that hybridize to the converted DNA; the second signal is collectively generated from the second competing probes of the plurality of competing probe sets that hybridize to the converted DNA; the third signal is collectively generated from the third competing probes of the plurality of competing probe sets that hybridize to the converted DNA; and/or the further signals are collectively generated from further competing probes of the plurality of competing probe sets that hybridize to the converted DNA.

In some aspects, provided herein is a method of analyzing a methylation status of a region of interest in a deoxyribonucleic acid (DNA) comprising: providing a biological sample comprising converted DNA generated by converting the DNA, wherein the nucleotide sequence of the converted DNA is indicative of the methylation state of the DNA, and wherein the converted DNA comprises target residues indicative of the methylation state of corresponding cytosine residues in the region of interest, contacting the biological sample with one or more sequencing primers that hybridize to converted DNA target sequences in the converted DNA, and performing a sequencing reaction to generate: a first signal associated with target residues indicative of unmethylated cytosine at a plurality of cytosine residues in the region of interest; and/or a second signal associated with target residues indicative of methylated cytosine at a plurality of cytosine residues in the region of interest. In some aspects, provided herein is a method for interrogating methylation of a region of interest in a deoxyribonucleic acid (DNA) comprising: providing a biological sample comprising converted DNA generated by converting the DNA, wherein the nucleotide sequence of the converted DNA is indicative of the methylation state of the DNA, and wherein the converted DNA comprises target residues indicative of the methylation state of corresponding cytosine residues in the region of interest, contacting the biological sample with one or more sequencing primers that hybridize to converted DNA target sequences in the converted DNA, and performing a sequencing reaction to generate: a first signal associated with target residues indicative of unmethylated cytosine at a plurality of cytosine residues in the region of interest; and/or a second signal associated with target residues indicative of methylated cytosine at a plurality of cytosine residues in the region of interest.

In some embodiments, the sequencing reaction comprises sequencing by synthesis and/or sequencing by ligation, and the sequencing reaction comprises generating a product from the one or more sequencing primers. In some embodiments, the sequencing reaction comprises an extension reaction that extends the one or more sequencing primers. In some embodiments, the sequencing reaction is a single-base extension reaction.

In some aspects, provided herein is a method of analyzing a methylation status of a region of interest in a deoxyribonucleic acid (DNA) comprising: providing a biological sample comprising converted DNA generated by converting the DNA, wherein the nucleotide sequence of the converted DNA is indicative of the methylation state of the DNA, and wherein the converted DNA comprises target residues indicative of the methylation state of corresponding cytosine residues in the region of interest, contacting the biological sample with a plurality of sequencing primers that hybridize to converted DNA target sequences that are adjacent and 3′ to target residues; performing an extension reaction that a) incorporates a first detectably labeled nucleotide into sequencing primers that hybridize 3′ to target residues indicative of unmethylated cytosines and/or b) incorporates a second detectably labeled nucleotide into sequencing primers that hybridize 3′ to target residues indicative of methylated cytosines; detecting a first signal associated with incorporation of the first detectably labeled nucleotide and/or detecting a second signal associated with the second detectably labeled nucleotide; and using the first signal and/or second signal to analyze the methylation status of the region of interest. In some aspects, provided herein is a method for interrogating methylation of a region of interest in a deoxyribonucleic acid (DNA) comprising: providing a biological sample comprising converted DNA generated by converting the DNA, wherein the nucleotide sequence of the converted DNA is indicative of the methylation state of the DNA, and wherein the converted DNA comprises target residues indicative of the methylation state of corresponding cytosine residues in the region of interest, contacting the biological sample with a plurality of sequencing primers that hybridize to converted DNA target sequences that are adjacent and 3′ to target residues; performing an extension reaction that a) incorporates a first detectably labeled nucleotide into sequencing primers that hybridize 3′ to target residues indicative of unmethylated cytosines and/or b) incorporates a second detectably labeled nucleotide into sequencing primers that hybridize 3′ to target residues indicative of methylated cytosines; and detecting a first signal associated with incorporation of the first detectably labeled nucleotide and/or detecting a second signal associated with the second detectably labeled nucleotide. In some embodiments, the method comprises using the first signal and/or second signal to analyze the methylation status of the region of interest. In some embodiments, the sequencing primers hybridize to converted DNA target sequences that are immediately 3′ to target residues. In some embodiments, the extension reaction is a single-base extension reaction.

In some embodiments, the first signal corresponds collectively to the target residues indicative of unmethylated cytosine at the plurality of cytosine residues in the region of interest; and/or the second signal corresponds collectively to the target residues indicative of methylated cytosine at the plurality of cytosine residues in the region of interest. In some embodiments, the first signal corresponds collectively to at least 10, 20, 50, or 100 target residues indicative of unmethylated cytosine at the plurality of cytosine residues in the region of interest; and/or the second signal corresponds collectively to at least 10, 20, 50, or 100 target residues indicative of methylated cytosine at the plurality of cytosine residues in the region of interest. In some embodiments, the first signal is collectively generated from the first detectably labeled nucleotides that are incorporated in the extension reaction; and/or the second signal is collectively generated from the second detectably labeled nucleotides that are incorporated in the extension reaction.

In some embodiments, the region of interest is at least 200 bases, at least 500 bases, or at least 1000 bases in length. In some embodiments, the method comprises contacting the biological sample with at least 10, 20, 50, or 100 sequencing primers.

In some embodiments, a non-cytosine target residue is indicative of unmethylated cytosine at the corresponding cytosine residue in the region of interest, and/or a cytosine target residue is indicative of methylated cytosine at the corresponding cytosine residue in the region of interest. In some embodiments, a non-cytosine target residue is indicative of methylated cytosine at the corresponding cytosine residue in the region of interest, and/or a cytosine target residue is indicative of unmethylated cytosine at the corresponding cytosine residue in the region of interest. In some embodiments, the non-cytosine target residue is uracil or dihydrouracil.

In some embodiments, the method comprises measuring size, intensity, and/or abundance of the first signal, the second signal, and/or a reference signal. In some embodiments, the method comprises comparing the first signal and/or second signal to the reference signal. In some embodiments, analyzing the methylation status of the region of interest comprises measuring size, intensity, and/or abundance of the first signal, the second signal, and/or a reference signal. In some embodiments, analyzing the methylation status of the region of interest in the DNA comprises comparing the first signal and/or second signal to the reference signal.

In some embodiments, increased size, intensity, and/or abundance of the first signal is indicative of the region of interest having a methylation status with a decreased proportion of cytosines that are methylated; and/or increased size, intensity, and/or abundance of the first signal in comparison to the reference signal is indicative of the region of interest having a methylation status with a decreased proportion of cytosines that are methylated. In some embodiments, increased size, intensity, and/or abundance of the second signal is indicative of the region of interest having a methylation status with an increased proportion of cytosines that are methylated; and/or increased size, intensity, and/or abundance of the second signal in comparison to the reference signal is indicative of the region of interest having a methylation status with an increased proportion of cytosines that are methylated.

In some embodiments, the method comprises comparing the first signal to the second signal. In some embodiments, analyzing the methylation status of the region of interest in the DNA comprises comparing the first signal to the second signal. In some embodiments, increased size, intensity, and/or abundance of the first signal in comparison to the second signal is indicative of the region of interest having a methylation status with a decreased proportion of cytosines that are methylated.

In some embodiments, the first and/or second signal is amplified. In some embodiments, one or more of the sequencing primers are independently selected from the group consisting of: a probe not comprising an overhang, a probe comprising a 5′ overhang; and a circularizable probe or probe set. In some embodiments, one or more of the sequencing primers comprise a barcode region associated with: a) the region of interest, b) the target residue to which the sequencing primer hybridizes immediately 3′ to, and/or c) the converted DNA target sequence that the sequencing primer hybridizes to.

In some embodiments, the DNA is genomic DNA. In some embodiments, the method comprises converting the DNA to generate the converted DNA.

In some embodiments, the method comprises determining a spatially localized position of the region of interest in the biological sample. In some embodiments, the methylation status of the region of interest is analyzed at the spatially localized position of the region of interest in the biological sample. In some embodiments, determining the spatially localized position of the region of interest comprises contacting the biological sample with one or more detectably-labeled probes that hybridize to the region of interest in the DNA and detecting a signal associated with the one or more detectably-labeled probes at the spatially localized position. In some embodiments, determining the spatially localized position of the region of interest comprises contacting the sample with one or more detectably-labeled probes that hybridize to a converted region of interest resulting from conversion of the DNA of the region of interest and detecting a signal associated with the one or more detectably-labeled probes at the spatially localized position.

In some embodiments, the one or more detectably-labeled probes comprise one or more of the competing probes; one or more of the methylation-state-specific probes; or one or more of the sequencing primers. In some embodiments, the one or more detectably-labeled probes do not comprise the competing probes; the methylation-state-specific probes; or the sequencing primers. In some embodiments, the spatially localized position of the region of interest is determined prior to generation of the converted DNA.

In some embodiments, the spatially localized position of the region of interest is determined after generation of the converted DNA. In some embodiments, the spatially localized position of the region of interest is in a cell in a tissue. In some embodiments, the spatially localized position of the region of interest is in a nucleus in a cell in a tissue.

In some embodiments, the region of interest is between about 100 bases and 1 kilobase, between about 1 kilobase and about 15 kilobases, between about 15 kilobases and about 25 kilobases, between about 25 kilobases and about 50 kilobases, between about 50 kilobases and about 75 kilobases, between about 75 kilobases and about 100 kilobases, or more than 100 kilobases in length. In some embodiments, the region of interest is at least 200 bases in length. In some embodiments, the region of interest is at least 500 bases in length. In some embodiments, the region of interest is at least 1000 bases in length. In some embodiments, the methylation status of the region of interest is, or is indicative of, the proportion of cytosine residues that are methylated in the region of interest. In some embodiments, the methylation status of the region of interest is, or is indicative of, the proportion of CpG cytosine residues that are methylated in the region of interest.

In some embodiments, the region of interest is a first region of interest, and the methylation status of a second region of interest is analyzed. In some embodiments, the first and second region of interest are at the same genomic locus on different chromosomes. In some embodiments, the first and second region of interest are at the same genomic locus in different cells. In some embodiments, the first and second region of interest are at different genomic loci.

In some embodiments, the biological sample is non-homogenized. In some embodiments, the biological sample is selected from the group consisting of a formalin-fixed, paraffin-embedded (FFPE) sample, a frozen tissue sample, and a fresh tissue sample. In some embodiments, the biological sample is fixed. In some embodiments, the biological sample is not fixed. In some embodiments, the biological sample is permeabilized. In some embodiments, the biological sample is embedded in a matrix. In some embodiments, wherein the matrix comprises a hydrogel. In some embodiments, the biological sample is cleared. In some embodiments, the method comprises clearing the biological sample. In some embodiments, clearing the biological sample comprises contacting the biological sample with a proteinase. In some embodiments, the biological sample is crosslinked. In some embodiments, the biological sample is a tissue slice between about 1 μm and about 50 μm in thickness. In some embodiments, the tissue slice is between about 5 μm and about 35 μm in thickness.

In some embodiments, converting the DNA comprises converting cytosines in the DNA to a different nucleotide, and wherein converting cytosines in the DNA is methylation-state-dependent. In some embodiments, cytosines that are not methylated are converted to a different nucleotide. In some embodiments, cytosines that are not methylated are converted to uracil. In some embodiments, cytosines that are methylated are converted to a different nucleotide. In some embodiments, cytosines that are methylated are converted to dihydrouracil. In some embodiments, cytosines that are methylated comprise methylated and/or hydroxymethylated cytosines. In some embodiments, converting the DNA comprises bisulfite conversion. In some embodiments, the bisulfite conversion comprises contacting the sample with a bisulfite reagent. In some embodiments, the bisulfite reagent is sodium bisulfite or ammonium bisulfite. In some embodiments, converting the DNA comprises enzymatic conversion. In some embodiments, converting the DNA comprises converting a first nucleotide in the DNA to a different nucleotide, wherein the converting depends on the methylation state of the first nucleotide. In some embodiments, the first nucleotide is an unmethylated cytosine residue and the different nucleotide is uracil. In some embodiments, converting the unmethylated cytosine residue in the DNA to uracil comprises contacting the sample with a cytidine or cytosine deaminase. In some embodiments, the cytidine or cytosine deaminase converts unmethylated cytosine residues and methylcytosine residues to uracil. In some embodiments, prior to contacting the sample with the cytidine or cytosine deaminase, the method comprises contacting the sample with a methylcytosine dioxygenase enzyme that catalyzes the conversion of methylcytosine residues to hydroxymethylcytosine residues. In some embodiments, hydroxymethylcytosine residues are not converted to uracil.

In some embodiments, converting unmethylated cytosine residues in the DNA to uracil comprises performing bisulfite conversion. In some embodiments, methylcytosine and/or hydroxymethylcytosine residues are not converted to uracil. In some embodiments, performing bisulfite conversion comprises contacting the sample with a bisulfite reagent. In some embodiments, the bisulfite reagent is sodium bisulfite or ammonium bisulfite. In some embodiments, contacting the sample with the bisulfite reagent leads to deamination of unmethylated cytosine to produce uracil, and does not lead to conversion of methylcytosine to uracil.

In some embodiments, the first nucleotide is a methylcytosine or hydroxymethylcytosine residue and the different nucleotide is dihyrdrouracil (DHU), and unmethylated cytostine residues in the DNA are not converted to uracil. In some embodiments, converting the first nucleotide to the different nucleotide comprises converting methylcytosine and/or hydroxymethylcytosine residues in the DNA to dihyrdrouracil by oxidation using ten-eleven translocation (TET) enzyme to 5-carboxylcytosine (5caC) prior to reduction to DHU.

In some aspects, provided herein is a kit for interrogating methylation of a region of interest in a deoxyribonucleic acid (DNA) in a biological sample, wherein the biological sample comprises converted DNA generated by converting the DNA, wherein the nucleotide sequence of the converted DNA is indicative of the methylation state of the DNA, wherein the kit comprises a plurality of methylation-state-specific probes, wherein each methylation-state-specific probe is complementary to a converted DNA target sequence indicative of a methylation state of a target sequence in the region of interest, and wherein the plurality of methylation state-specific probes collectively target a plurality of converted DNA target sequences corresponding to a plurality of target sequences in the region of interest.

In some embodiments, the plurality of methylation state-specific probes are configured to be used to generate a methylation-state-specific signal associated with hybridization of methylation-state-specific probes to the converted DNA. In some embodiments, the plurality of methylation-state-specific probes is a plurality of first methylation-state-specific probes; each first methylation-state-specific probe is complementary to a converted DNA target sequence indicative of a first methylation state of a target sequence in the region of interest; and the methylation-state-specific signal is a first methylation-state-specific signal. In some embodiments, the kit further comprises a plurality of second methylation-state-specific probes, wherein each second methylation-state-specific probe is complementary to a converted DNA target sequence indicative of a second methylation state of a target sequence in the region of interest, and wherein the plurality of second methylation state-specific probes collectively target a plurality of converted DNA target sequences corresponding to a plurality of target sequences in the region of interest. In some embodiments, the second methylation state-specific probes are configured to be used to generate a second methylation-state-specific signal associated with hybridization of second methylation-state-specific probes to the converted DNA. In some embodiments, the plurality of converted DNA target sequences targeted by the first methylation state-specific probes and the plurality of converted DNA target sequences targeted by the second methylation state-specific probes correspond to the same plurality of target sequences in the region of interest. In some embodiments, the plurality of converted DNA target sequences targeted by the first methylation state-specific probes and the plurality of converted DNA target sequences targeted by the second methylation state-specific probes do not correspond to the same plurality of target sequences in the region of interest. In some embodiments, the plurality of methylation-state-specific probes comprises at least 3, 5, 10, 20, 50, 100, or 500 methylation-state-specific probes; the plurality of first methylation-state-specific probes comprises at least 3, 5, 10, 20, 50, 100, or 500 first methylation-state-specific probes; and/or the plurality of second methylation-state-specific probes comprises at least 3, 5, 10, 20, 50, 100, or 500 second methylation-state-specific probes. In some embodiments, each target sequence in the region of interest comprises 1, 2, 3, 4, or more cytosine residues. In some embodiments, each target sequence in the region of interest comprises 1, 2, 3, 4, or more CpG cytosine residues. In some embodiments, the methylation-state-specific probes are directly or indirectly associated with a detectable label; the first methylation-state-specific probes are directly or indirectly associated with a first detectable label; and/or the second methylation-state-specific probes are directly or indirectly associated with a second detectable label. In some embodiments, the kit comprises the detectable label, the first detectable label, and/or the second detectable label. In some embodiments, the kit comprises a reagent for converting the DNA. In some embodiments, the biological sample is a cell or a tissue sample.

In some aspects, provided herein is a kit for interrogating methylation of a region of interest in a deoxyribonucleic acid (DNA) in a biological sample, wherein the biological sample comprises converted DNA generated by converting the DNA, wherein the nucleotide sequence of the converted DNA is indicative of the methylation state of the DNA, and wherein the kit comprises a plurality of competing probe sets, each competing probe set comprising: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest. In some embodiments, the plurality of competing probe sets are configured to be used to generate a first signal associated with hybridization of first competing probes of the plurality of competing probe sets to the converted DNA. In some embodiments, the plurality of competing probe sets are configured to be used to generate a second signal associated with hybridization of second competing probes of the plurality of competing probe sets to the converted DNA. In some embodiments, one or more competing probe sets of the plurality of competing probe sets further comprise a third competing probe that is complementary to a third converted DNA target sequence indicative of a third methylation state of the target sequence in the region of interest. In some embodiments, the plurality of competing probe sets are configured to be used to generate a third signal associated with hybridization of third competing probes of the plurality of competing probe sets to the converted DNA. In some embodiments, one or more competing probe sets of the plurality of competing probe sets comprise further competing probes that are complementary to further converted DNA target sequences indicative of further methylation states of the target sequence in the region of interest. In some embodiments, the plurality of competing probe sets are configured to be used to generate further signals associated with hybridization of further competing probes of the plurality of competing probe sets to the converted DNA. In some embodiments, the first signal corresponds to the first methylation states of the target sequences; the second signal corresponds to the second methylation states of the target sequences; the third signal corresponds to the third methylation states of the target sequences; and/or the further signals correspond to further methylation states of the target sequences. In some embodiments, the plurality of competing probe sets comprises: a first competing probe set comprising competing probes complementary to converted DNA target sequences indicative of different methylation states of a first target sequence in the region of interest; and a second competing probe set comprising competing probes complementary to converted DNA target sequences indicative of different methylation states of a second target sequence in the region of interest. In some embodiments, the plurality of competing probe sets further comprises a third competing probe set comprising competing probes complementary to converted DNA target sequences indicative of different methylation states of a third target sequence in the region of interest. In some embodiments, the plurality of competing probe sets comprises further competing probe sets, each further competing probe set comprising competing probes complementary to converted DNA target sequences indicative of different methylation states of further target sequences in the region of interest. In some embodiments, the plurality of competing probe sets comprises at least 10, 20, 50, 100, or 500 competing probe sets for at least 10, 20, 50, 100, or 500 target sequences in the region of interest, respectively. In some embodiments, a competing probe set is provided for each of the target sequences in the region of interest. In some embodiments, the first, second, third, and/or further methylation states of the target sequences in the region of interest each comprise a different proportion of methylated CpG cytosine residues. In some embodiments, each target sequence in the region of interest comprises 1, 2, 3, 4, or more cytosine residues, and/or wherein each target sequence in the region of interest comprises 1, 2, 3, 4, or more CpG cytosine residues. In some embodiments, each target sequence in the region of interest is independently between 10 and 50 nucleotides in length. In some embodiments, the first methylation states of the target sequences in the region of interest comprise fewer methylated cytosines than the second methylation states of the target sequences in the region of interest; the first methylation states of the target sequences in the region of interest comprise a smaller proportion of methylated CpG cytosine residues than the second methylation states of the target sequences in the region of interest; the first methylation states are methylation states in which none of the CpG cytosine residues in the target sequences in the region of interest are methylated; and/or the second methylation states are methylation states in which all of the CpG cytosine residues in the target sequences in the region of interest are methylated. In some embodiments, the first methylation states of the target sequences in the region of interest comprise more methylated cytosines than the second methylation states of the target sequences in the region of interest; the first methylation states of the target sequences in the region of interest comprise a larger proportion of methylated CpG cytosine residues than the second methylation states of the target sequences in the region of interest; the first methylation states are methylation states in which all of the CpG cytosine residues in the target sequences in the region of interest are methylated; and/or the second methylation states are methylation states in which none of the CpG cytosine residues in the target sequences in the region of interest are methylated. In some embodiments, the first competing probes of the plurality of competing probe sets are directly or indirectly associated with a first detectable label corresponding to the first methylation state; the second competing probes of the plurality of competing probe sets are directly or indirectly associated with a second detectable label corresponding to the second methylation state; and/or the third and/or further competing probes of the plurality of competing probe sets are directly or indirectly associated with third and/or further detectable labels corresponding to third and/or further methylation states. In some embodiments, the kit comprises the first detectable label, second detectable label, third detectable label, and/or further detectable labels. In some embodiments, the first detectable label, second detectable label, third detectable label, and/or further detectable labels are fluorophores. In some embodiments, the kit comprises a reagent for converting the DNA. In some embodiments, the biological sample is a cell or a tissue sample.

In some aspects, provided herein is a kit for interrogating methylation of a region of interest in a deoxyribonucleic acid (DNA) in a biological sample, wherein the biological sample comprises converted DNA generated by converting the DNA, wherein the nucleotide sequence of the converted DNA is indicative of the methylation state of the DNA, and wherein the converted DNA comprises target residues indicative of the methylation state of corresponding cytosine residues in the region of interest, and wherein the kit comprises a plurality of sequencing primers that hybridize to converted DNA target sequences that are adjacent and 3′ to target residues. In some embodiments, the sequencing primers hybridize to converted DNA target sequences that are immediately 3′ to target residues. In some embodiments, the kit further comprises a first detectably labeled nucleotide that is configured to be incorporated into sequencing primers that hybridize 3′ to target residues indicative of unmethylated cytosines, and/or a second detectably labeled nucleotide that is configured to be incorporated into sequencing primers that hybridize 3′ to target residues indicative of methylated cytosines. In some embodiments, the first detectably labeled nucleotide is configured to be incorporated in a single-base extension reaction into sequencing primers that hybridize 3′ to target residues indicative of unmethylated cytosines, and/or the second detectably labeled nucleotide is configured to be incorporated in a single-base extension reaction into sequencing primers that hybridize 3′ to target residues indicative of methylated cytosines. In some embodiments, the first detectably labeled nucleotide is configured to be used to generate a first signal that corresponds to the target residues indicative of unmethylated cytosine at the plurality of cytosine residues in the region of interest, and wherein the second detectably labeled nucleotide is configured to be used to generate a second signal that corresponds to the target residues indicative of methylated cytosine at the plurality of cytosine residues in the region of interest. In some embodiments, the kit comprises at least 10, 20, 50, 100, or 500 sequencing primers. In some embodiments, the region of interest is at least 200 bases, at least 500 bases, at least 1000 bases, or at least 5,000 bases in length. In some embodiments, the kit comprises a reagent for converting the DNA. In some embodiments, the biological sample is a cell or a tissue sample.

In some aspects, provided herein is a system, comprising: a biological sample comprising converted DNA generated by converting DNA, wherein the nucleotide sequence of the converted DNA is indicative of the methylation state of the DNA, and wherein the DNA comprises a region of interest; and a plurality of methylation-state-specific probes, wherein each methylation-state-specific probe is complementary to a converted DNA target sequence indicative of a methylation state of a target sequence in the region of interest, and wherein the plurality of methylation state-specific probes collectively target a plurality of converted DNA target sequences corresponding to a plurality of target sequences in the region of interest. In some embodiments, the plurality of methylation-state-specific probes are configured to be used to generate a methylation-state-specific signal associated with hybridization of methylation-state-specific probes to the converted DNA. In some embodiments, the system further comprises an apparatus for detecting the methylation-state-specific signal in the biological sample. In some embodiments, the methylation-state-specific signal is an optical signal at a location in the biological sample.

In some aspects, provided herein is a system, comprising: a biological sample comprising converted DNA generated by converting DNA, wherein the nucleotide sequence of the converted DNA is indicative of the methylation state of the DNA, and wherein the DNA comprises a region of interest; and a plurality of competing probe sets, each competing probe set comprising: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest. In some embodiments, the plurality of competing probe sets are configured to be used to generate a first signal associated with hybridization of first competing probes of the plurality of competing probe sets to the converted DNA. In some embodiments, the plurality of competing probe sets are configured to be used to generate a second signal associated with hybridization of second competing probes of the plurality of competing probe sets to the converted DNA. In some embodiments, the system further comprises an apparatus for detecting the first signal and/or second signal. In some embodiments, the first signal and/or second signal is an optical signal detected at a location in the biological sample.

In some aspects, provided herein is a system, comprising: a biological sample comprising converted DNA generated by converting DNA, wherein the nucleotide sequence of the converted DNA is indicative of the methylation state of the DNA, and wherein the converted DNA comprises target residues indicative of the methylation state of corresponding cytosine residues in a region of interest of the DNA; and a plurality of sequencing primers that hybridize to converted DNA target sequences that are adjacent and 3′ to target residues. In some embodiments, the sequencing primers hybridize to converted DNA target sequences that are immediately 3′ to target residues. In some embodiments, the system further comprises a first detectably labeled nucleotide that is configured to be incorporated into sequencing primers that hybridize 3′ to target residues indicative of unmethylated cytosines, and/or a second detectably labeled nucleotide that is configured to be incorporated into sequencing primers that hybridize 3′ to target residues indicative of methylated cytosines. In some embodiments, the first detectably labeled nucleotide is configured to be incorporated in a single-base extension reaction into sequencing primers that hybridize 3′ to target residues indicative of unmethylated cytosines, and/or the second detectably labeled nucleotide is configured to be incorporated in a single-base extension reaction into sequencing primers that hybridize 3′ to target residues indicative of methylated cytosines. In some embodiments, the first detectably labeled nucleotide is configured to be used to generate a first signal that corresponds to the target residues indicative of unmethylated cytosine at the plurality of cytosine residues in the region of interest, and wherein the second detectably labeled nucleotide is configured to be used to generate a second signal that corresponds to the target residues indicative of methylated cytosine at the plurality of cytosine residues in the region of interest. In some embodiments, the system comprises at least 10, 20, 50, 100, or 500 sequencing primers. In some embodiments, the system further comprises an apparatus for detecting the first signal and/or second signal. In some embodiments, the first signal and/or second signal is an optical signal at a location in the biological sample. In some embodiments, the region of interest is at least 200 bases, at least 500 bases, at least 1000 bases, or at least 5,000 bases in length. In some embodiments, the biological sample is a cell or a tissue sample.

### BRIEF DESCRIPTION OF THE DRAWINGS

The drawings illustrate certain features and advantages of this disclosure. These embodiments are not intended to limit the scope of the appended claims in any manner.

FIG. 1 shows schematics illustrating an exemplary method for generating a methylation-state-specific signal for a region of interest.

FIGS. 2A-2B show schematics illustrating an exemplary method for generating signals corresponding to different methylation states in a region of interest using competing probes.

FIG. 3 shows a schematic illustrating exemplary hybridization of a competing probe set at a converted DNA target sequence that is indicative of a target sequence in the region of interest having an unmethylated (left) or methylated (right) CpG cytosine.

FIGS. 4A-4B show schematics illustrating an exemplary method for generating signals corresponding to different methylation states of cytosine residues in a region of interest using sequencing primers and incorporation of detectably labeled nucleotides.

FIG. 5 shows schematics illustrating exemplary incorporation of detectably labeled nucleotides that are complementary to a target residue that is indicative of a cytosine residue in a region of interest that is unmethylated (left) or methylated (right).

FIG. 6 shows schematics illustrating an exemplary workflow for in situ analysis of DNA methylation at multiple regions of interest.

FIGS. 7A-7D show schematics illustrating exemplary arrangements of a detectable label associated with a probe that is hybridized to a converted DNA target sequence.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

The ability to effectively analyze DNA methylation can provide valuable information about gene expression, cell identity, and disease. For example, epigenomic studies indicate that cytosine methylation is a reliable marker for regulatory elements such as enhancers and promoters (He, Y. et al., PNAS. 2017. 114(9):E1633-E1640). Single-cell DNA methylation sequencing can be used to identify cell types and infer gene expression from identification of differentially methylated regions (Liu, H. et al., Nature. 2021. 598(7879):120-128; Mattesen, T. B. et al., Clin. Epigenetics. 2021. 13 (1): 20).

Despite the rich source of information that DNA methylation can provide, DNA methylation profiling is not as widely utilized as other methods, such as gene expression profiling, for understanding cellular states. This is in part because of challenges associated with current technologies for analysis of DNA methylation. For example, there are comparatively fewer technologies that exist to profile DNA methylation at a single-cell resolution, and especially for profiling DNA methylation in a spatial context. The cost of DNA methylation profiling using sequencing is also higher than RNA sequencing. This is because DNA methylation profiling typically requires whole genome sequencing (WGS), and when applied at the single-cell level, can require profiling of thousands of genomes of individual cells.

Although WGS at single base resolution is the gold standard for DNA methylation profiling, in a typical single-cell DNA methylation experiment, the data analysis comprises “binning,” e.g. collapsing the base level information into 1-100 kilobase (kb) bins (Liu, H. et al., Nature. 2021. 598 (7879): 120-128; Luo, C. et al., Nat. Commun. 2018. 9 (1): 3824). As a result, a great deal of resources are used to produce single-base resolution data when only kilobase level information is analyzed. If a technology existed to produce methylation information over large (e.g. kilobase-sized) regions with greater efficiency or reduced cost, this would greatly increase the capacity to profile DNA methylation across many cells, and increase access to DNA methylation profiling for researchers with fewer resources.

Provided herein are methods that provide an approach for analyzing methylation information over one or more region of interest of DNA. In some aspects, the methods provide a readout that is representative of the methylation status of the region of interest of DNA as a whole. In some aspects, the methods are analogous to “binning” in that they provide overall methylation information for a region of interest (e.g. a 1 kilobase-sized region of interest), rather than at single-base resolution. The readout, for example, can comprise a detected signal, wherein the strength of the detected signal corresponds to a proportion of cytosines (such as a proportion of CpG cytosines) that are methylated across a region of interest. For example, a signal of increasing intensity can correspond to a region of interest having an increased proportion of cytosines that are methylated, optionally in comparison to a reference signal, or in comparison to a comparable signal generated from another region of interest (e.g. a region of interest at the same genetic locus but in a different cell). In embodiments, the detected signal can be a methylation-state-specific signal. In some aspects, the methylation-state-specific signal is generated collectively from a plurality of interrogated sites in the region of interest that share a particular methylation state (such as from a plurality of cytosines that are methylated). In some embodiments, the readout can comprise more than one signal (e.g. more than one methylation-state-specific signal), wherein the strength of each signal corresponds to a different methylation state. For example, a first signal can be generated collectively from a plurality of interrogated sites in the region of interest that are methylated, and a second signal can be generated collectively from a plurality of interrogated sites in the region of interest that are unmethylated. In this example, increased intensity of the first signal can correspond to the proportion of cytosines in the region of interest that are methylated, and increased intensity of the second signal can correspond to the proportion of cytosines in the region of interest that are unmethylated.

In some aspects, the methods provided herein allow for analysis of methylation at the level of a region of interest in DNA, rather than analysis at the individual base level, thus providing valuable information about epigenetic states while also increasing efficiency and facilitating in-situ and/or spatial analysis of methylation. It can be seen that the methods provided herein meet challenges associated with current methods for DNA methylation profiling. In some aspects, generating methylation information from a plurality of target sequences and/or cytosine residues in a region of interest improves accuracy of the method (e.g., by reducing the impact of error at the single-base level).

In some embodiments, to determine the methylation status of a region of interest, a biological sample comprising the region of interest (such as a tissue section) is subjected to methylation-dependent nucleotide conversion (also referred to as converting the DNA, or DNA conversion, herein). For example, using bisulfite conversion, double-stranded genomic DNA is converted to single-stranded DNA fragments, and unmethylated cytosine nucleotides are converted to uracil, whereas methylated cytosines are not converted to uracil. The resulting sequence of the converted DNA is indicative of the methylation state of the DNA, and can be interrogated using the methods described herein. Various alternative methods of methylation-dependent nucleotide conversion are also compatible with the methods described herein, such as enzymatic conversion. Different methods of methylation-dependent nucleotide conversion can produce converted DNA having a sequence with a different correspondence to the sequence of the DNA. For example, in some methods (e.g. bisulfite conversion) unmethylated cytosines in the DNA are converted to non-cytosine residues in the converted DNA, whereas in other methods (e.g. certain enzymatic conversion methods), methylated cytosines in the DNA are converted to non-cytosine residues in the converted DNA. In the various instances of methylation-dependent DNA conversion, the sequence of the converted DNA corresponds to and/or is indicative of the methylation state of the DNA. In some aspects, the methods described herein rely on interrogation of converted DNA to determine the methylation state of DNA in a corresponding region of interest. In some embodiments, the method comprises providing a biological sample comprising converted DNA generated by converting the DNA. In some embodiments, the method comprises converting the DNA.

Various methods are provided herein for analyzing methylation status at the level of a region of interest, each of which is described in more detail herein. In the methods outlined above and herein, an average or relative methylation status is determined for one or more regions of interest (e.g. 1-kb-sized regions of interest), which is comparable to the resolution of information that is typically analyzed in single-cell sequencing methylation assays. The information can be obtained in many cells for a fraction of the cost of single-cell sequencing assays for analyzing DNA methylation, and can further include spatial information, which is not provided in other DNA methylation assays.

In some embodiments, provided herein is a method for interrogating and/or analyzing the methylation status of a region of interest in a deoxyribonucleic acid (DNA) using methylation-state-specific probes. In some aspects, provided herein is a method of interrogating and/or analyzing a methylation status of a region of interest in a deoxyribonucleic acid (DNA). In some embodiments, the method comprises providing a biological sample comprising converted DNA generated by converting the DNA. In some embodiments, the nucleotide sequence of the converted DNA is indicative of the methylation state of the DNA. In some embodiments, the method comprises contacting the biological sample with a plurality of methylation-state-specific probes. In some embodiments, each methylation-state-specific probe is complementary to a converted DNA target sequence indicative of a methylation state of a target sequence in the region of interest. In some embodiments, the plurality of methylation state-specific probes collectively target a plurality of converted DNA target sequences corresponding to a plurality of target sequences in the region of interest. In some embodiments, the method comprises detecting a methylation-state-specific signal associated with hybridization of methylation-state-specific probes to the converted DNA. In some embodiments, the method comprises using the methylation-state-specific signal to analyze the methylation status of the region of interest. In some embodiments, the methylation-state-specific signal is collectively generated from the methylation-state-specific probes that hybridize to the converted DNA.

In some embodiments, provided herein is a method for interrogating and/or analyzing the methylation status of a region of interest in a deoxyribonucleic acid (DNA) using competing probes. In some aspects, provided herein is a method of interrogating and/or analyzing a methylation status of a region of interest in a deoxyribonucleic acid (DNA). In some embodiments, the method comprises providing a biological sample comprising converted DNA generated by converting the DNA. In some embodiments, the nucleotide sequence of the converted DNA is indicative of the methylation state of the DNA. In some embodiments, the method comprises contacting the biological sample with a plurality of competing probe sets. In some embodiments, each (or one or more) competing probe set comprises: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest. In some embodiments, the method comprises detecting a first signal associated with hybridization of first competing probes of the plurality of competing probe sets to the converted DNA. In some embodiments, the method comprises using the first signal to analyze the methylation status of the region of interest in the DNA.

In some embodiments, provided herein is a method for interrogating and/or analyzing the methylation status of a region of interest in a deoxyribonucleic acid (DNA) using sequencing primers. In some aspects, provided herein is a method of interrogating and/or analyzing a methylation status of a region of interest in a deoxyribonucleic acid (DNA). In some embodiments, the method comprises providing a biological sample comprising converted DNA generated by converting the DNA. In some embodiments, the nucleotide sequence of the converted DNA is indicative of the methylation state of the DNA. In some embodiments, the converted DNA comprises target residues indicative of the methylation state of corresponding cytosine residues in the region of interest. In some embodiments, the method comprises contacting the biological sample with a plurality of sequencing primers that hybridize to converted DNA target sequences that are adjacent and 3′ to target residues (e.g. one or more of the target residues). In some embodiments, the method comprises performing an extension reaction that incorporates a first detectably labeled nucleotide into sequencing primers that hybridize 3′ to target residues indicative of unmethylated cytosines. In some embodiments, the method comprises performing an extension reaction that incorporates a second detectably labeled nucleotide into sequencing primers that hybridize 3′ to target residues indicative of methylated cytosines. In some embodiments, the method comprises performing an extension reaction that a) incorporates a first detectably labeled nucleotide into sequencing primers that hybridize 3′ to target residues indicative of unmethylated cytosines and/or b) incorporates a second detectably labeled nucleotide into sequencing primers that hybridize 3′ to target residues indicative of methylated cytosines. In some embodiments, the method comprises detecting a first signal associated with incorporation of the first detectably labeled nucleotide. In some embodiments, the method comprises detecting a second signal associated with the second detectably labeled nucleotide. In some embodiments, the method comprises using the first signal to analyze the methylation status of the region of interest. In some embodiments, the method comprises using the second signal to analyze the methylation status of the region of interest. In some embodiments, the method comprises using the first signal and the second signal to analyze the methylation status of the region of interest. In some embodiments, the sequencing primers hybridize to converted DNA target sequences that are immediately 3′ to target residues (e.g. one or more of the target residues). In some embodiments, the sequencing primers hybridize to converted DNA target sequences that are immediately 3′ to the target residues indicative of unmethylated cytosines and/or methylated cytosines. In some embodiments, the extension reaction is a single-base extension reaction. In some embodiments, the first signal corresponds collectively to the target residues indicative of unmethylated cytosine at the plurality of cytosine residues in the region of interest. In some embodiments, the second signal corresponds collectively to the target residues indicative of methylated cytosine at the plurality of cytosine residues in the region of interest.

In some aspects, also provided herein are kits, systems, and compositions related to the methods provided herein.

### II. Probes and Assays for Analysis of DNA Methylation

Provided herein are compositions and methods for interrogating and/or analyzing DNA methylation in a biological sample. In some aspects, the methods (e.g. interrogation and/or analysis) are performed in situ in a biological sample. In some embodiments, the analysis comprises analyzing the methylation status of a region of interest of DNA. In some aspects, the DNA is converted to generate converted DNA in which the sequence of the converted DNA is indicative of the methylation state of the DNA (e.g. by bisulfite conversion or enzymatic conversion). In some aspects, the converted DNA is contacted with probes that hybridize to specific converted DNA target sequences indicative of methylation states of corresponding target sequences in the region of interest. Various schemes and assays involving such probes are described in the sections below. In some aspects, the methods allow for the collective generation of one or more signals corresponding to the methylation states of many target sequences in the region of interest to provide an overall readout of the methylation status of the region of interest. In some aspects, the collective generation of signals for an entire region of interest increases the utility, efficiency, and robustness of the assay, for example in comparison to assays in which interrogation and/or analysis of methylation is performed at the level of individual nucleotides. Exemplary assays and methods for generating signals from and/or analyzing the methylation status of a region of interest are described further below.

### A. Methylation-State-Specific Probes for Analyzing Methylation Status

In some embodiments, provided herein is a method. In some embodiments, provided herein is a method for interrogating methylation of a region of interest in a deoxyribonucleic acid (DNA). In some embodiments, provided herein is a method for interrogating methylation of a region of interest in a deoxyribonucleic acid (DNA) using methylation-state-specific probes. In some embodiments, provided herein is a method for interrogating the methylation status of a region of interest in a deoxyribonucleic acid (DNA) using methylation-state-specific probes. In some embodiments, interrogating the methylation comprises contacting a biological sample with methylation-state-specific probes. In some embodiments, interrogating the methylation comprises detecting one or more methylation-state-specific signals. In some aspects, interrogating the methylation does not need to comprise downstream analysis of detected methylation-state-specific signals. In some embodiments, provided herein is a method for generating a signal in a biological sample. In some embodiments, provided herein is a method for detecting a signal in a biological sample. In some embodiments, provided herein is a method for detecting a methylation-state-specific signal. In some embodiments, provided herein is a method for detecting a methylation-state-specific signal in a biological sample. In some embodiments, provided herein is a method for analyzing the methylation status of a region of interest in a deoxyribonucleic acid (DNA) using methylation-state-specific probes. In some embodiments, the method comprises providing a biological sample comprising converted DNA generated by converting the DNA, wherein the nucleotide sequence of the converted DNA is indicative of the methylation state of the DNA. In some embodiments, the method comprises contacting a biological sample comprising the converted DNA with a plurality of methylation-state-specific probes. In some embodiments, one or more of the methylation-state-specific probes is complementary to a converted DNA target sequence indicative of a methylation state of a target sequence in the region of interest. In some embodiments, each methylation-state-specific probe is complementary to a converted DNA target sequence indicative of a methylation state of a target sequence in the region of interest. For example, the methylation-state-specific probes can each be complementary to a converted DNA target sequence indicative of a methylation state of a target sequence in which all of the CpG cytosines (cytosines in a CpG context) in the target sequence are methylated. In this example, if the region of interest has a high methylation status (e.g., is highly methylated), then the converted DNA will comprise many converted DNA target sequences complementary to the methylation-state-specific probes, and consequently many of the methylation-state-specific probes will hybridize to the converted DNA. Conversely, if the region of interest has a low methylation status (e.g., a methylation status in which a small proportion of CpG cytosines are methylated), then the converted DNA will comprise few converted DNA target sequences complementary to the methylation-state-specific probes, and consequently few of the methylation-state-specific probes will hybridize to the converted DNA. After allowing the methylation-state-specific probes to hybridize, the method can comprise detecting a methylation-state-specific signal associated with hybridization of methylation-state-specific probes to the converted DNA. In some embodiments, the method further comprises using the methylation-state-specific signal to analyze the methylation status of the region of interest. The methylation-state-specific signal can be detected, for example, by detecting detectable labels (e.g. fluorophores) that are directly or indirectly associated with the methylation-state-specific probes. It can be seen in the above-described example that the strength of the signal will correlate with the degree of methylation in the region of interest; a larger proportion of CpG cytosines in the region of interest that are methylated will result in a stronger signal, whereas a smaller proportion of CpG cytosines in the region of interest that are methylated will result in a weaker signal. In some embodiments, the methylation-state-specific signal is collectively generated from the methylation-state-specific probes that hybridize to the converted DNA. In this way, a single methylation-state-specific signal can represent the collective methylation status of the region of interest, which is based on the methylation states of the plurality of target sequences in the region of interest. Collective generation of the methylation-state-specific signal from a plurality of methylation-state-specific probes further provides a mechanism for generating a signal that is robust to non-specific binding of individual methylation-state-specific probes.

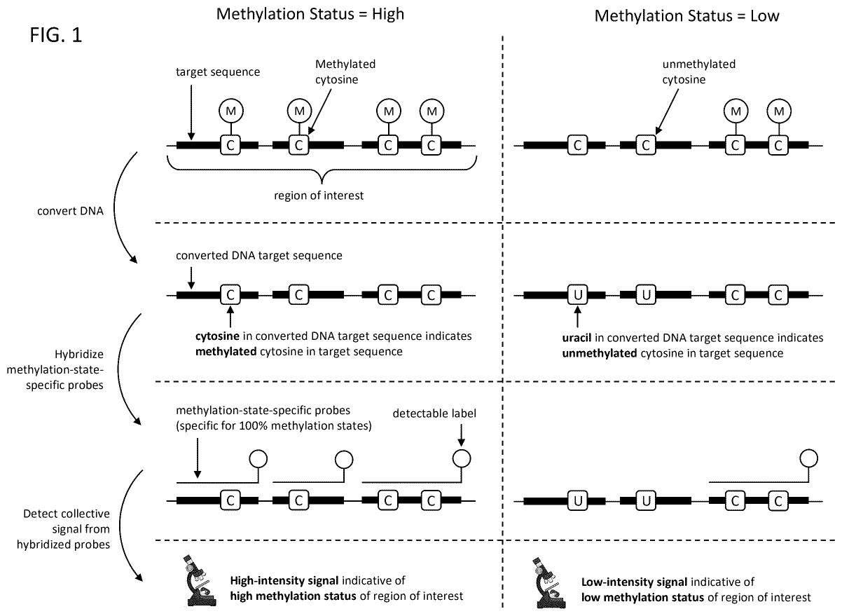

FIG. 1 illustrates an exemplary embodiment of a method that utilizes methylation-state-specific probes for interrogating or analyzing the methylation status of a region of interest in DNA. Scenarios with relatively high methylation status (left) and relatively low methylation status (right) are shown. The region of interest in the DNA comprises a plurality of target sequences, each comprising one or more cytosines that may be methylated or unmethylated. DNA is converted to generate converted DNA comprising converted DNA target sequences indicative of the methylation states of the target sequences in the region of interest. In this example, cytosine in converted DNA is indicative of methylated cytosine at the corresponding residue in the region of interest, and uracil in converted DNA is indicative of unmethylated cytosine at the corresponding residue in the region of interest. After converting the DNA, methylation-state-specific probes are hybridized to converted DNA target sequences. In this example, the methylation-state-specific probes are complementary to converted DNA target sequences indicative of target sequences in the region of interest in which all of the illustrated cytosines (e.g. which may be all cytosines, or all CpG cytosines) are methylated. The methylation state-specific probes can be associated with a detectable label. A methylation-state-specific signal associated with hybridization of methylation-state-specific probes to the converted DNA is generated (e.g. from the detectable label associated with the methylation-state-specific probes). If a large proportion of the target sequences are fully methylated (e.g. all of the CpG cytosines in a target sequence are methylated), then a large proportion of the methylation-state-specific probes will hybridize to converted DNA target sequences in the converted DNA (e.g. as shown on the left). Conversely, if a small proportion of the target sequences are fully methylated, then a small proportion of the methylation-state-specific probes will hybridize to converted DNA target sequences in the converted DNA (e.g. as shown on the right). Consequently, the methylation-state-specific signal can be used to analyze the methylation status of the region of interest; in this example, a stronger (e.g. higher intensity) methylation-state-specific signal is indicative of higher methylation status (e.g. a higher proportion of methylated cytosines), and a weaker (e.g. lower intensity) methylation-state-specific signal is indicative of lower methylation status (e.g. a lower proportion of methylated cytosines in the region of interest). FIG. 1 illustrates the method in which a single set of methylation-state-specific probes are used, which correspond to methylation states in which all cytosines in a target sequence are methylated. The methylation-state-specific probes can alternatively be specific for any other methylation state (e.g. in which none of the cytosines or CpG cytosines in the target sequences are methylated, or in which a defined proportion of cytosines or CpG cytosines in the target sequences are methylated). In some embodiments, multiple sets of methylation-state-specific probes (e.g. first methylation-state-specific probes corresponding to a first methylation state and second methylation-state-specific probes corresponding to a second methylation state) may be used to generate more than one methylation-state-specific signal, which may be analyzed (e.g. a first methylation-state-specific signal and a second methylation-state-specific signal.

In some aspects, provided herein is a method for interrogating or analyzing a methylation status of a region of interest in a deoxyribonucleic acid (DNA). In some embodiments, the method comprises providing a biological sample comprising converted DNA generated by converting the DNA. In some embodiments, the nucleotide sequence of the converted DNA is indicative of the methylation state of the DNA. In some embodiments, the method comprises contacting the biological sample with a plurality of methylation-state-specific probes. In some embodiments, each methylation-state-specific probe is complementary to a converted DNA target sequence indicative of a methylation state of a target sequence in the region of interest. In some embodiments, the plurality of methylation state-specific probes collectively target a plurality of converted DNA target sequences corresponding to a plurality of target sequences in the region of interest. In some embodiments, the method comprises detecting a methylation-state-specific signal associated with hybridization of methylation-state-specific probes to the converted DNA. In some embodiments, the method comprises detecting a methylation-state-specific signal associated with hybridization of one or more of the methylation-state-specific probes to the converted DNA. In some embodiments, the method comprises using the methylation-state-specific signal to analyze the methylation status of the region of interest. In some embodiments, the methylation-state-specific signal is collectively generated from the methylation-state-specific probes that hybridize to the converted DNA.

In some embodiments, the plurality of methylation-state-specific probes is a plurality of first methylation-state-specific probes. In some embodiments, each (or one or more) first methylation-state-specific probe is complementary to a converted DNA target sequence indicative of a first methylation state of a target sequence in the region of interest. In some embodiments, the methylation-state-specific signal is a first methylation-state-specific signal. In some embodiments, the method comprises detecting the first methylation-state-specific signal associated with hybridization of first methylation-state-specific probes to the converted DNA. In some embodiments, the first methylation-state-specific signal corresponds to and/or is indicative of the methylation status of the region of interest. In some embodiments, the method comprises using the first methylation-state-specific signal to analyze the methylation status of the region of interest. In some embodiments, the method further comprises contacting the biological sample with a plurality of second methylation-state-specific probes. In some embodiments, each (or one or more) second methylation-state-specific probe is complementary to a converted DNA target sequence indicative of a second methylation state of a target sequence in the region of interest. In some embodiments, the plurality of second methylation state-specific probes collectively target a plurality of converted DNA target sequences corresponding to a plurality of target sequences in the region of interest. In some embodiments, the method comprises detecting a second methylation-state-specific signal associated with hybridization of second methylation-state-specific probes to the converted DNA. In some embodiments, the method comprises detecting a second methylation-state-specific signal associated with hybridization of one or more of the second methylation-state-specific probes to the converted DNA. In some embodiments, the second methylation-state-specific signal corresponds to and/or is indicative of the methylation status of the region of interest. In some embodiments, the method comprises using the second methylation-state-specific signal to analyze the methylation status of the region of interest. In some embodiments, the first methylation-state-specific signal is collectively generated from the first methylation-state-specific probes that hybridize to the converted DNA. In some embodiments, the second methylation-state-specific signal is collectively generated from the second methylation-state-specific probes that hybridize to the converted DNA. In some embodiments, the first methylation-state-specific signal and second methylation-state-specific signal correspond to and/or are indicative of the methylation status of the region of interest, alone or in combination.

In some embodiments, the plurality of converted DNA target sequences targeted by the first methylation state-specific probes and the plurality of converted DNA target sequences targeted by the second methylation state-specific probes correspond to the same plurality of target sequences in the region of interest. Thus, in some aspects, a first methylation-state-specific probe and second methylation-state-specific probe can together constitute a competing probe set, e.g. as described in section II.B. Similarly, the first methylation-state-specific probes and second methylation-state-specific probes can together constitute a plurality of competing probe sets, e.g. as described in section II.B. In some embodiments, the plurality of converted DNA target sequences targeted by the first methylation state-specific probes and the plurality of converted DNA target sequences targeted by the second methylation state-specific probes do not correspond to the same plurality of target sequences in the region of interest, or correspond to partially overlapping target sequences in the region of interest. Thus, first methylation-state-specific probes and second methylation-state-specific probes may, but do not need to constitute competing probe sets.

In some embodiments, the region of interest is at least 200 bases, at least 500 bases, or at least 1000 bases in length. In some embodiments, the plurality of methylation-state-specific probes comprises at least 3, 5, 10, 20, 50, 100, or 500 methylation-state-specific probes. In some embodiments, plurality of first methylation-state-specific probes comprises at least 3, 5, 10, 20, 50, 100, or 500 first methylation-state-specific probes. In some embodiments, the plurality of second methylation-state-specific probes comprises at least 3, 5, 10, 20, 50, 100, or 500 second methylation-state-specific probes. In some embodiments, the plurality of methylation-state-specific probes comprises at most 3, 5, 10, 20, 50, 100, or 500 methylation-state-specific probes. In some embodiments, plurality of first methylation-state-specific probes comprises at most 3, 5, 10, 20, 50, 100, or 500 first methylation-state-specific probes. In some embodiments, the plurality of second methylation-state-specific probes comprises at most 3, 5, 10, 20, 50, 100, or 500 second methylation-state-specific probes. In some embodiments, each target sequence in the region of interest comprises 1, 2, 3, 4, or more cytosine residues. In some embodiments, each target sequence in the region of interest comprises 1, 2, 3, 4, or more CpG cytosine residues. In some embodiments, each target sequence in the region of interest is independently between 10 and 50 nucleotides in length. In some embodiments, one or more target sequences in the region of interest is independently between 10 and 50 nucleotides in length. In some embodiments, analyzing the methylation status of the region of interest comprises measuring the size, intensity, and/or abundance of the methylation-state-specific signal, the first methylation-state-specific signal, the second methylation-state-specific signal, and/or a reference signal. In some embodiments, analyzing the methylation status of the region of interest in the DNA comprises comparing the methylation-state-specific signal, the first methylation-state-specific signal, and/or the second methylation-state-specific signal to the reference signal. In some embodiments, the method comprises measuring the size, intensity, and/or abundance of the methylation-state-specific signal, the first methylation-state-specific signal, the second methylation-state-specific signal, and/or a reference signal. In some embodiments, the method comprises comparing the methylation-state-specific signal, the first methylation-state-specific signal, and/or the second methylation-state-specific signal to the reference signal. In some embodiments, the method comprises comparing any two of: the methylation-state-specific signal, the first methylation-state-specific signal, the second methylation-state-specific signal, and a reference signal.

In some embodiments, increasing size, intensity, and/or abundance of the first methylation-state-specific signal is indicative of a methylation status of the region of interest that is increasingly similar to the first methylation states of the target sequences in the region of interest. In some embodiments, increasing size, intensity, and/or abundance of the first methylation-state-specific signal in comparison to the reference signal is indicative of a methylation status of the region of interest that increasingly similar to the first methylation states of the target sequences in the region of interest.

In some embodiments, analyzing the methylation status of the region of interest in the DNA comprises comparing the first methylation-state-specific signal to the second methylation-state-specific signal. In some embodiments, the method comprises comparing the first methylation-state-specific signal to the second methylation-state-specific signal. In some embodiments, increasing size, intensity, and/or abundance of the first methylation-state-specific signal in comparison to the second methylation-state-specific signal is indicative of a methylation status of the region of interest that is increasingly more similar to the first methylation states of the target sequences in the region of interest than to the second methylation states of the target sequences in the region of interest.

In some embodiments, increased size, intensity, and/or abundance of the first methylation-state-specific signal in comparison to a reference signal or to the second methylation-state-specific signal is indicative of a methylation status of the region of interest in which a lower proportion of cytosine residues are methylated. For example, in some embodiments, the first methylation states of the target sequences in the region of interest comprise fewer methylated cytosines than the second methylation states of the target sequences in the region of interest; the first methylation states of the target sequences in the region of interest comprise a smaller proportion of methylated CpG cytosine residues than the second methylation states of the target sequences in the region of interest; the first methylation states are methylation states in which none of the CpG cytosine residues in the target sequences in the region of interest are methylated; and/or the second methylation states are methylation states in which all of the CpG cytosine residues in the target sequences in the region of interest are methylated.

In some embodiments, increased size, intensity, and/or abundance of the first methylation-state-specific signal in comparison to a reference signal or to the second methylation-state-specific signal is indicative of a methylation status of the region of interest in which a higher proportion of cytosine residues are methylated. For example, in some embodiments, the first methylation states of the target sequences in the region of interest comprise more methylated cytosines than the second methylation states of the target sequences in the region of interest; the first methylation states of the target sequences in the region of interest comprise a larger proportion of methylated CpG cytosine residues than the second methylation states of the target sequences in the region of interest; the first methylation states are methylation states in which all of the CpG cytosine residues in the target sequences in the region of interest are methylated; and/or second methylation states are methylation states in which none of the CpG cytosine residues in the target sequences in the region of interest are methylated.

In some embodiments, the methylation-state-specific probes are directly or indirectly associated with a detectable label, and detecting the methylation-state-specific signal comprises detecting the detectable label. In some embodiments, the first methylation-state-specific probes are directly or indirectly associated with a first detectable label, and detecting the first methylation-state-specific signal comprises detecting the first detectable label. In some embodiments, the second methylation-state-specific probes are directly or indirectly associated with a second detectable label, and detecting the second methylation-state-specific signal comprises detecting the second detectable label. In some embodiments, the methylation-state-specific probes are directly associated with the detectable label, the first methylation-state-specific probes are directly associated with the first detectable label, and/or the second methylation-state-specific probes are directly associated with the second detectable label.

In some embodiments, the methylation-state-specific probes are configured to directly or indirectly bind and/or hybridize to detectably labeled probes comprising the detectable label. In some embodiments, the first methylation-state-specific probes are configured to directly or indirectly bind and/or hybridize to detectably labeled probes comprising the first detectable label. In some embodiments, the second methylation-state-specific probes are configured to directly or indirectly bind and/or hybridize to detectably labeled probes comprising the second detectable label. Any suitable detectable label(s) may be used. In some embodiments, the detectable label, the first detectable label, and/or the second detectable label are optical labels. In some embodiments, the detectable label, the first detectable label, and/or the second detectable label are fluorophores.

In some embodiments, one or more of the methylation-state-specific probes, first methylation-state-specific probes, and/or second methylation-state-specific probes are independently selected from the group consisting of: a probe not comprising an overhang, a probe comprising a 3′ and/or 5′ overhang; a circular probe; and a circularizable probe or probe set. In some embodiments, the methylation-state-specific probes, first methylation-state-specific probes, and/or second methylation-state-specific probes comprise a 3′ overhang.

In some embodiments, one or more of the methylation-state-specific probes, first methylation-state-specific probes, and/or second methylation-state-specific probes comprise a barcode region associated with: a) the region of interest, b) the target sequence in the region of interest corresponding to the converted DNA target sequence to which it hybridizes, and/or c) the methylation state of the target sequence in the region of interest corresponding to the converted DNA target sequence to which it hybridizes. In some embodiments, the barcode region can be used to determine the identity of the region of interest (e.g. if different regions of interest corresponding to different genomic coordinates are being analyzed in the biological sample). In some embodiments, the barcode region can be used to determine the spatially localized position of the region of interest in the biological sample. In some embodiments, the barcode region can be used to generate any of the methylation-state-specific signals. For example, the barcode region can comprise a sequence that corresponds to the same methylation state that the methylation-state-specific probe corresponds to, and the sequence can be hybridized by an oligonucleotide that is directly or indirectly associated with a detectable label corresponding to the methylation state. Thus, it can be seen that a single methylation-state-specific probe can comprise a barcode region enabling identification and localization of the region of interest, as well as methylation analysis.

In some embodiments, the methylation-state-specific signal, the first methylation-state-specific signal, and/or the second methylation-state-specific signal is amplified. In some embodiments, the signal amplification comprises using the methylation-state-specific probes, first methylation-state-specific probes, and/or second methylation-state-specific probes to perform: rolling circle amplification (RCA); hybridization chain reaction (HCR); linear oligonucleotide hybridization chain reaction (LO-HCR); primer exchange reaction (PER); assembly of branched structures; hybridization of a plurality of detectable probes directly or indirectly on the methylation-state-specific probes, first methylation-state-specific probes, and/or second methylation-state-specific probes or products thereof; or any combination thereof. This disclosure makes reference to corresponding components used in the described methods. It can be seen that various components of the method have a clear correspondence to one another. For example, in some embodiments, each methylation-state-specific probe corresponds to a target sequence in the region of interest. In some embodiments, each methylation-state-specific probe also corresponds to a methylation state (which the converted DNA target sequence to which the methylation-state-specific probe is complementary is indicative of). In some embodiments, each methylation-state-specific signal generated from hybridized methylation-state-specific probes in turn corresponds to the methylation state for which the methylation-state-specific probe is specific. In some embodiments, each detectable label used to generate a methylation-state-specific signal in turn corresponds to a methylation state.

### B. Competing Probe Sets for Analyzing Methylation Status

In some embodiments, provided herein is a method. In some embodiments, provided herein is a method for interrogating methylation of a region of interest in a deoxyribonucleic acid (DNA). In some embodiments, provided herein is a method for interrogating methylation of a region of interest in a deoxyribonucleic acid (DNA) using a plurality of competing probe sets. In some embodiments, provided herein is a method for interrogating the methylation status of a region of interest in a deoxyribonucleic acid (DNA) using a plurality of competing probe sets. In some embodiments, interrogating the methylation comprises contacting a biological sample with a plurality of competing probe sets. In some embodiments, interrogating the methylation comprises detecting one or more signals. In some aspects, interrogating the methylation does not need to comprise downstream analysis of detected signals. In some embodiments, provided herein is a method for generating a signal in a biological sample. In some embodiments, provided herein is a method for detecting a signal in a biological sample. In some embodiments, provided herein is a method for analyzing the methylation status of a region of interest in a deoxyribonucleic acid (DNA) using a plurality of competing probe sets. In some embodiments, the method comprises providing a biological sample comprising converted DNA generated by converting the DNA, wherein the nucleotide sequence of the converted DNA is indicative of the methylation state of the DNA. In some embodiments, the method comprises contacting a biological sample comprising the converted DNA with a plurality of competing probe sets. In some embodiments, each (or one or more) competing probe set comprises a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest. In some embodiments, one or more of the competing probe sets comprises a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest. In some embodiments, the first and second converted DNA target sequences are indicative of different methylation states of the same target sequence in the region of interest. Thus, in a given molecule of converted DNA, only one of the first and second converted DNA target sequence targeted by a first and second competing probe of a competing probe set will be present, depending on the methylation state of the target sequence in the region of interest. It can be seen that in some embodiments, the first and second converted DNA target sequences will be identical to one another, with the exception of residues in the converted DNA target sequences that correspond to cytosines that are differentially methylated between the first and second methylation states (e.g. methylated in the first methylation state of the target sequence and unmethylated in the second methylation state of the target sequence). Under hybridization conditions (e.g. stringent hybridization conditions), the first competing probe will preferentially hybridize to the converted DNA when the first converted DNA target sequence corresponding to the first methylation state is present, and the second competing probe will preferentially hybridize to the converted DNA when the second converted DNA target sequence corresponding to the second methylation state is present. The method can comprise detecting signals associated with hybridization of first competing probes and/or second competing probes of the plurality of competing probe sets to the converted DNA. For example, the method can comprise detecting a first signal associated with hybridization of first competing probes of the plurality of competing probes, and/or detecting a second signal associated with hybridization of second competing probes of the plurality of competing probes.

In an exemplary embodiment, the first competing probes are complementary to converted DNA target sequences indicative of first methylation states of target sequences in the region of interest, wherein the first methylation states are methylation states in which none of the CpG cytosine residues in the target sequences in the region of interest are methylated. In this example, the strength (e.g. size, intensity, and/or abundance) of a first signal associated with hybridization of first competing probes would correlate with the degree of methylation in the region of interest; a smaller proportion of CpG cytosines in the region of interest that are methylated will result in a stronger first signal. In some aspects, the first signal can be collectively generated from the first competing probes that hybridize to the converted DNA. In this way, a single first signal can represent the collective methylation status of the region of interest, which is based on the methylation states of the plurality of target sequences in the region of interest targeted by the plurality of competing probe sets.

In some embodiments, the second competing probes can be complementary to converted DNA target sequences indicative of second methylation states of target sequences in the region of interest, wherein the second methylation states are methylation states in which all of the CpG cytosine residues in the target sequences in the region of interest are methylated. In this example, the strength (e.g. size, intensity, and/or abundance) of a second signal associated with hybridization of second competing probes would correlate with the degree of methylation in the region of interest; a higher proportion of CpG cytosines in the target sequences of the region of interest that are methylated will result in a stronger second signal. In some aspects, the second signal can be collectively generated from the second competing probes that hybridize to the converted DNA. In this way, a single second signal can represent the collective methylation status of the region of interest, which is based on the methylation states of the plurality of target sequences in the region of interest targeted by the plurality of competing probe sets.

Collective generation of signal(s) from a plurality of competing probe sets further provides a mechanism for generating signals that are robust to non-specific binding of individual competing probes, or individual instances in which the non-complementary competing probe of a competing probe set hybridizes instead of the complementary competing probe.

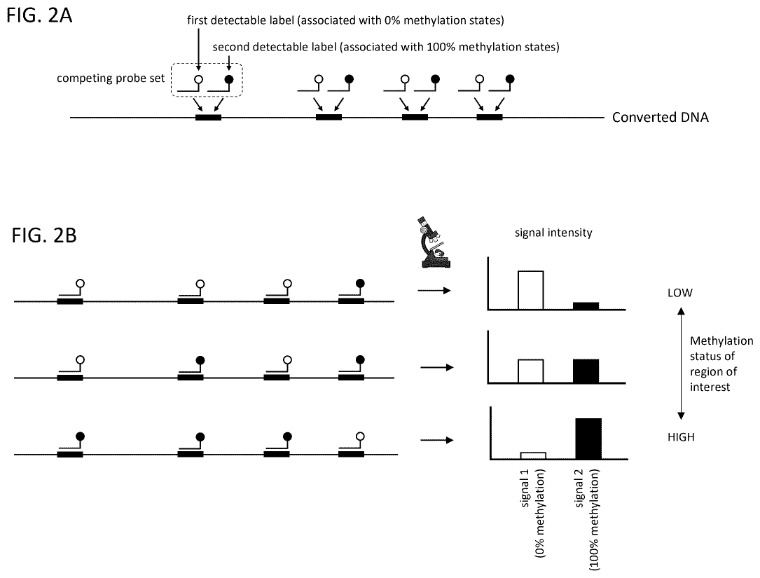

An exemplary embodiment of a method that utilizes competing probe sets for interrogating and/or analyzing the methylation status of a region of interest is illustrated in FIGS. 2A-2B. FIG. 2A shows converted DNA generated from a region of interest of DNA in a biological sample. The converted DNA comprises a plurality of converted DNA target sequences, each corresponding to a target sequence in the region of interest in the DNA. The biological sample is contacted with a plurality of competing probe sets, which, as shown in the figure, can each comprise: a first competing probe that is complementary to a first converted DNA target sequence indicative of a first methylation state of a target sequence in the region of interest; and a second competing probe that is complementary to a second converted DNA target sequence indicative of a second methylation state of the target sequence in the region of interest. Only one of the first converted DNA target sequence and second converted DNA target sequence will be present in the converted DNA molecule, depending on whether the first methylation state or second methylation state was present in the target sequence in the region of interest. The competing probes of a competing probe set will compete for the converted DNA target sequence, and only one of the competing probes will be perfectly complementary to the DNA target sequence, and will preferentially hybridize, in particular under stringent conditions. Thus, hybridization of first competing probes is indicative of first methylation states in the region of interest, and hybridization of second competing probes to converted DNA is indicative of second methylation states in the region of interest. Methylation-state-specific detectable labels can be associated with competing probes (and by extension, specific methylation states). As shown in FIG. 2A, first detectable labels can be associated with the first competing probes of the plurality of competing probe sets, which in turn can be associated with first methylation states, such as methylation states in which none of the CpG cytosines in the target sequence are methylated. In addition, second detectable labels can be associated with the second competing probes of the plurality of competing probe sets, which in turn can be associated with second methylation states, such as methylation states in which all of the CpG cytosines in the target sequence are methylated. FIG. 2B shows competing probes hybridized to converted DNA in three different scenarios ranging from low methylation status (top); to high methylation status (bottom). In the low methylation status scenario (top), a greater number of first competing probes are hybridized to the converted DNA than second competing probes. Consequently, a first signal generated from the first detectable label is relatively strong (e.g. in comparison to the second signal or reference signal), whereas a second signal generated from the second detectable label is relatively weak (e.g. in comparison to the first signal or a reference signal). In the high methylation status scenario, a greater number of second competing probes are hybridized to the converted DNA than first competing probes. Consequently, a second signal generated from the second detectable label is relatively strong (e.g. in comparison to the first signal or reference signal), whereas a first signal generated from the first detectable label is relatively weak (e.g. in comparison to the second signal or a reference signal). It can be seen that the methylation status can be analyzed based on the strength of the first signal and/or second signal. In some embodiments, both the first and second signal can be used in the analysis of methylation status. However, it is possible to exclude either the first or second signal from analysis, and rely on either the first signal or the second signal for analyzing methylation status (e.g. by comparing the first or second signal to a reference signal).

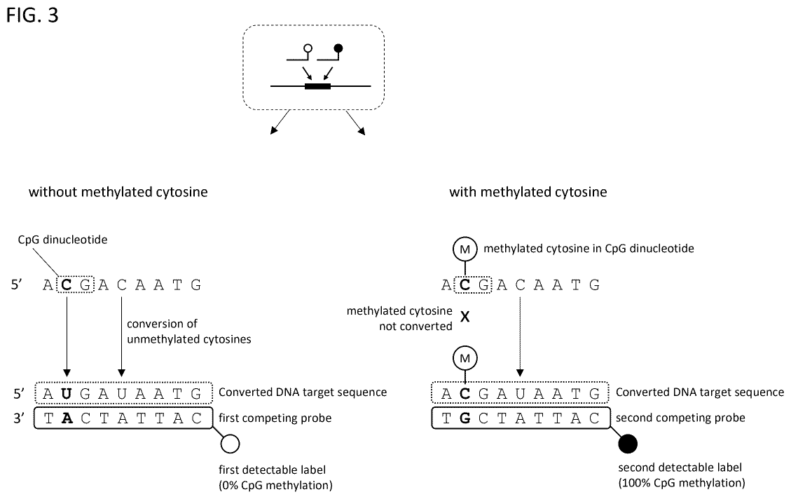

FIG. 3 shows a schematic illustrating hybridization of competing probes of a single competing probe set to converted DNA target sequences that correspond to different methylation states of the same target sequence in a region of interest. For purposes of illustration, in this example, the converted DNA is generated by converting unmethylated cytosines to uracil, and not converting methylated cytosines to a different nucleotide (e.g. by bisulfite sequencing). The competing probe set comprises a first competing probe and second competing probe. The first competing probe is complementary to a first converted DNA target sequence indicative of a first methylation state of a target sequence in the region of interest in which none of the CpG cytosines are methylated. The second competing probe is complementary to a second converted DNA target sequence indicative of a second methylation state of the target sequence in the region of interest in which all of the CpG cytosines are methylated. The first competing probe preferentially hybridizes when the first methylation state is present in the target sequence (left) and the second competing probe preferentially hybridizes when the second methylation state is present in the target sequence (right). Hybridization of the first competing probe or second competing probe can be detected, for example, by first and second detectable labels that can be directly or indirectly associated with the first and second competing probes, respectively.

In some embodiments, a target sequence can comprise a single potentially methylated cytosine (e.g. a single CpG cytosine in a human genome context, such as shown in FIG. 3). Such a target sequence can be interrogated with a set of two competing probes, wherein the first competing probe corresponds to 0% methylation of CpGs in the target sequence, and the second competing probe corresponds to 100% methylation of CpGs in the target sequence. In some embodiments, however, a target sequence comprises 2, 3, 4, or more CpG cytosine residues. In some embodiments, any of the 2, 3, 4, or more CpG cytosine residues is potentially methylated. In some embodiments, the competing probe set for a given target sequence comprises a plurality of competing probes corresponding to a plurality of converted DNA target sequences indicative of different methylation states of the target sequence. In some embodiments, the competing probes of the competing probe set that correspond to the same percentage of methylated cytosines are directly or indirectly associated with the same detectable label. In some embodiments, competing probes can correspond to methylation of any suitable proportion of cytosine residues in a target sequence.

In a particular example, in a given 1 kb region of interest, the region of interest may be broken down into 50 different 20 nucleotide target sequences. Within any one of those target sequences, there may be, for example, four different CpG cytosines. An individual target sequence can then be targeted with a set of competing probes corresponding to 0%, 25%, 50%, 75%, or 100% of the CpG cytosines in the target sequence. In some embodiments, all permutations of methylation states for a given target sequence (e.g., 0%, 25%, 50%, 75%, or 100%) can be assigned to different detectable labels (e.g., different fluorescent labels). For example, 0% methylation of the target sequence could correspond to an ATTO488 labeled competing probe (or a competing probe comprising an overhang for binding of an ATTO488 labeled probe), 25% methylation of the target sequence could correspond to a ATTO532 labeled competing probe (or a competing probe comprising an overhang for binding of an ATTO532 labeled probe), 50% methylation of the target sequence could correspond to a dual-labeled ATTO488 and ATTO647 competing probe (or a competing probe comprising an overhang for binding of dual-labeled ATTO488 and ATTO647 probe), 75% methylation of the target sequence could correspond to an ATTO590 labeled competing probe (or a competing probe comprising an overhang for binding of an ATTO590 probe), and 100% methylation of the target sequence could correspond to an ATTO647 labeled competing probe (or a competing probe comprising an overhang for binding of an ATTO647 probe).

In some embodiments, different competing probes or methylation-state-specific probes can be provided for any or all possible permutations of a given methylation state for a target sequence. In some embodiments, all possible permutations of a given methylation state for the target sequence are associated with the same detectable label. For example, a single competing probe or methylation-state-specific probe for a target sequence can be provided corresponding to a methylation level of 0% (e.g., methylation of 0 out of 4 potentially methylated cytosines in the target sub-region). Similarly, a single competing probe or methylation-state-specific probe for the target sequence can be provided corresponding to a methylation level of 100% (e.g., methylation of 4 out of 4 potentially methylated cytosines in the target sub-region). In contrast, 4 different competing probes (or methylation-state-specific probes) can be provided corresponding to a methylation state of 25% methylation for a single target sequence (e.g., methylation of any one of 4 potentially methylated cytosines in the target sequence). The different competing probes or methylation-state-specific probes corresponding to the same methylation state (e.g., 25%) can be directly or indirectly associated with the same detectable label. Not all methylation states need to be interrogated by probes. For example, the method may be carried out with only methylation-state-specific probes for 0% and 100% methylation states for one, more, or all of the target sequences, even if one or more of the target sequences comprise more than two possible methylation states.

Each region of interest can contain a plurality of target sequences. In some embodiments, each competing probe set corresponds to a single target sequence, and different competing probes in a competing probe set are complementary to different converted DNA target sequences that are indicative of different methylation states of the target sequence. For example, a first competing probe can be complementary to a first converted DNA target sequence corresponding to 0% of the cytosines being methylated within the target sequence, a second competing probe can be complementary to a second converted DNA target sequence corresponding to 25% of the cytosines being methylated within the target sequence, and so on. Each competing probe can be associated with a detectable label (such as a fluorescent label, for example, ATTO488, ATTO532, ATTO647, ATTO590, ATTO647) that corresponds to the methylation state (e.g. 0%, 25%, 50%, 75%, 100% of cytosines methylated) that the competing probe corresponds to. For example, competing probes complementary to a converted DNA target sequence indicative of 0% of the cytosines being methylated within the target sequence can be associated with a first fluorophore (e.g. ATTO488), competing probes complementary to a converted DNA target sequence corresponding to 25% of the cytosines being methylated within the target sequence can be associated with a second fluorophore (e.g. ATTO532), and so on. In some embodiments, the correspondence of methylation state and detectable label is the same for all competing probe sets for the region of interest, such that a common signal corresponding to each (or one or more) interrogated methylation state is detected across the entire region of interest. The number of competing probes in a competing probe set can vary. The number of methylation states interrogated by a competing probe set can also vary.

In some embodiments, the competing probe sets are added together to the biological sample and hybridized under stringent conditions such that the perfectly complementary probes preferentially hybridize to the converted DNA target sequences. Some error can be tolerated in this process (e.g., hybridization of a competing probe that is not perfectly complementary), since multiple target sequences are assayed within a region of interest to generate a collective signal. One or more wash steps can be performed to remove unhybridized probes. The sample is then imaged to detect signals associated with hybridized competing probes associated with different methylation states (e.g. via detectable labels associated with the competing probes). The signals corresponding to different methylation states are used to analyze the overall methylation status of the region of interest (or multiple regions of interest).

In some aspects, provided herein is a method for interrogating and/or analyzing a methylation status of a region of interest in a deoxyribonucleic acid (DNA). In some embodiments, the method comprises providing a biological sample comprising converted DNA generated by converting the DNA. In some embodiments, the nucleotide sequence of the converted DNA is indicative of the methylation state of the DNA. In some embodiments, the method comprises contacting the biological sample with a plurality of competing probe sets. In some embodiments, each competing probe set comprises: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest. In some embodiments, the method comprises detecting a first signal associated with hybridization of first competing probes of the plurality of competing probe sets to the converted DNA. In some embodiments, the method comprises using the first signal to analyze the methylation status of the region of interest in the DNA.

In some aspects, interrogation and/or analysis of the methylation status of the region of interest does not require generation of a second signal. For example, the first signal generated from first competing probes can be used to analyze the methylation status of the region of interest. The intensity of the first signal can be used, for example, to compare the methylation status of the region of interest in one cell to a second region of interest in a second cell, or to a reference signal. Thus, in some embodiments, only one of the competing probes in a competing probe set is associated with a detectable label. In some embodiments, two competing probes in a competing probe set are associated with a detectable label. In some embodiments, each (or one or more) competing probe in a competing probe set is associated with a detectable label.

In some embodiments, the method further comprises detecting a second signal associated with hybridization of second competing probes of the plurality of competing probe sets to the converted DNA. In some embodiments, the method comprises using the first signal and second signal to analyze the methylation status of the region of interest in the DNA. In some embodiments, one or more competing probe sets of the plurality of competing probe sets further comprise a third competing probe that is complementary to a third converted DNA target sequence indicative of a third methylation state of the target sequence in the region of interest. In some embodiments, the method comprises detecting a third signal associated with hybridization of third competing probes of the plurality of competing probe sets to the converted DNA. In some embodiments, the method further comprises using the first signal, second signal, and third signal to analyze the methylation status of the region of interest in the DNA. In some embodiments, one or more competing probe sets of the plurality of competing probe sets comprise further (e.g. fourth, fifth, sixth, etc.) competing probes that are complementary to further converted DNA target sequences indicative of further methylation states of the target sequence in the region of interest. In some embodiments, the method further comprises detecting further (e.g. fourth, fifth, sixth, etc.) signals associated with hybridization of further competing probes of the plurality of competing probe sets to the converted DNA, and using the first signal, second signal, third signal, and/or further signals to analyze the methylation status of the region of interest in the DNA. In some embodiments, the first signal corresponds to the first methylation states of the target sequences. In some embodiments, second signal corresponds to the second methylation states of the target sequences. In some embodiments, the third signal corresponds to the third methylation states of the target sequences. In some embodiments, the further signals correspond to further (e.g. fourth, fifth, sixth, etc.) methylation states of the target sequences.

In some embodiments, the plurality of competing probe sets comprises: a first competing probe set comprising competing probes complementary to converted DNA target sequences indicative of different methylation states of a first target sequence in the region of interest. In some embodiments, the plurality of competing probe sets comprises a second competing probe set comprising competing probes complementary to converted DNA target sequences indicative of different methylation states of a second target sequence in the region of interest. In some embodiments, the plurality of competing probe sets further comprises a third competing probe set comprising competing probes complementary to converted DNA target sequences indicative of different methylation states of a third target sequence in the region of interest. In some embodiments, the plurality of competing probe sets comprises further (e.g. fourth, fifth, sixth, etc.) competing probe sets, each further competing probe set comprising competing probes complementary to converted DNA target sequences indicative of different methylation states of further (e.g. fourth, fifth, sixth, etc.) target sequences in the region of interest.

In some aspects, each competing probe set can comprise any suitable number of competing probes. For example, in some embodiments, a competing probe set comprises 2 competing probes. In some embodiments, a competing probe set comprises 3 competing probes. In some embodiments, a competing probe set comprises 4, 5, 6, 7, 8, 9, 10, or more competing probes. In some aspects, any suitable number of competing probe sets can be used. In some embodiments, the plurality of competing probe sets comprises at least 2 competing probe sets. In some embodiments, the plurality of competing probe sets comprises at least 3 competing probe sets. In some embodiments, the plurality of competing probe sets comprises at least 4 competing probe sets. In some embodiments, the plurality of competing probe sets comprises at least 5 competing probe sets. In some embodiments, the plurality of competing probe sets comprises at least 10 competing probe sets. In some embodiments, the plurality of competing probe sets comprises at least 20 competing probe sets. In some embodiments, the plurality of competing probe sets comprises at least 50, at least 100, or at least 500 competing probe sets. In some embodiments, each competing probe set is for (e.g. corresponds to and/or interrogates the methylation state of) a target sequence in the region of interest. In some embodiments, the plurality of competing probe sets comprises at least 10, 20, 50, 100, or 500 competing probe sets for at least 10, 20, 50, 100, or 500 target sequences in the region of interest, respectively. In some embodiments, a competing probe set is provided for each (or one or more) of the target sequences in the region of interest. In some embodiments, the plurality of competing probe sets comprises at most 5 competing probe sets. In some embodiments, the plurality of competing probe sets comprises at most 10 competing probe sets. In some embodiments, the plurality of competing probe sets comprises at most 20 competing probe sets. In some embodiments, the plurality of competing probe sets comprises at most 50, at most 100, or at most 500 competing probe sets. In some embodiments, each competing probe set is for (e.g. corresponds to and/or interrogates the methylation state of) a target sequence in the region of interest. In some embodiments, the plurality of competing probe sets comprises at most 10, 20, 50, 100, or 500 competing probe sets for at most 10, 20, 50, 100, or 500 target sequences in the region of interest, respectively.

In some embodiments, the first, second, third, and/or further methylation states of the target sequences in the region of interest each comprise a different proportion of methylated CpG cytosine residues. In some embodiments, each target sequence in the region of interest comprises 1, 2, 3, 4, or more cytosine residues. In some embodiments, each target sequence in the region of interest comprises 1, 2, 3, 4, or more CpG cytosine residues. In some embodiments, each target sequence in the region of interest is independently between 10 and 50 nucleotides in length. In some embodiments, one or more target sequence in the region of interest is independently between 10 and 50 nucleotides in length.

In some embodiments, the method comprises measuring the first signal, second signal, third signal, further signals, and/or a reference signal. In some embodiments, interrogating and/or analyzing the methylation status of the region of interest comprises measuring the first signal, second signal, third signal, further signals, and/or a reference signal. In some embodiments, interrogating and/or analyzing the methylation status of the region of interest comprises measuring the first signal. In some embodiments, interrogating and/or analyzing the methylation status of the region of interest comprises measuring the second signal. In some embodiments, interrogating and/or analyzing the methylation status of the region of interest comprises measuring the third signal. In some embodiments, interrogating and/or analyzing the methylation status of the region of interest comprises measuring the further signals. In some embodiments, interrogating and/or analyzing the methylation status of the region of interest comprises measuring a reference signal. In some embodiments, the method comprises measuring the size, intensity, and/or abundance of the first signal, second signal, third signal, further signals, and/or a reference signal. In some embodiments, interrogating and/or analyzing the methylation status of the region of interest comprises measuring the size, intensity, and/or abundance of the first signal, second signal, third signal, further signals, and/or a reference signal. In some embodiments, interrogating and/or analyzing the methylation status of the region of interest comprises comparing at least two of: the first signal, second signal, third signal, further signals, and reference signal. In some embodiments, interrogating and/or analyzing the methylation status of the region of interest comprises comparing the first signal and the second signal. In some embodiments, interrogating and/or analyzing the methylation status of the region of interest comprises comparing the first signal and the reference signal. In some embodiments, interrogating and/or analyzing the methylation status of the region of interest comprises comparing the first signal, second signal, third signal, and/or further signals to the reference signal. In some embodiments, increasing size, intensity, and/or abundance of a detected signal is indicative of a methylation status of the region of interest that is increasingly similar to the methylation state to which the signal corresponds. For example, for a first signal generated from hybridized first probes complementary to converted DNA target sequences indicative of 0% methylation states (i.e. a first signal corresponding to 0% methylation states), an increasing intensity of the first signal is indicative of a methylation status in which a larger proportion of target sequences are unmethylated. In some embodiments, increasing size, intensity, and/or abundance of the first signal is indicative of a methylation status of the region of interest that is increasingly similar to the first methylation states of the target sequences in the region of interest. In some embodiments, increasing size, intensity, and/or abundance of the second signal is indicative of a methylation status of the region of interest that is increasingly similar to the second methylation states of the target sequences in the region of interest. In some embodiments, increasing size, intensity, and/or abundance of the first signal in comparison to the reference signal is indicative of a methylation status of the region of interest that increasingly similar to the first methylation states of the target sequences in the region of interest. In some embodiments, the method comprises comparing the first signal to the second signal. In some embodiments, interrogating and/or analyzing the methylation status of the region of interest comprises comparing the first signal to the second signal. In some embodiments, increasing size, intensity, and/or abundance of the first signal in comparison to the second signal is indicative of a methylation status of the region of interest that is increasingly similar to the first methylation states of the target sequences in the region of interest than to the second methylation states of the target sequences in the region of interest.

In particular embodiments, increased size, intensity, and/or abundance of the first signal in comparison to the second signal or to the reference signal can be indicative of a methylation status of the region of interest in which a lower proportion of cytosine residues are methylated. For example, in some particular embodiments, the first methylation states of the target sequences in the region of interest comprise fewer methylated cytosines than the second methylation states of the target sequences in the region of interest; the first methylation states of the target sequences in the region of interest comprise a smaller proportion of methylated CpG cytosine residues than the second methylation states of the target sequences in the region of interest; the first methylation states are methylation states in which none of the CpG cytosine residues in the target sequences in the region of interest are methylated; and/or the second methylation states are methylation states in which all of the CpG cytosine residues in the target sequences in the region of interest are methylated.

In particular embodiments, increased size, intensity, and/or abundance of the first signal in comparison to the second signal or to the reference signal is indicative of a methylation status of the region of interest in which a higher proportion of cytosine residues are methylated. For example, in some particular embodiments, the first methylation states of the target sequences in the region of interest comprise more methylated cytosines than the second methylation states of the target sequences in the region of interest; the first methylation states of the target sequences in the region of interest comprise a larger proportion of methylated CpG cytosine residues than the second methylation states of the target sequences in the region of interest; the first methylation states are methylation states in which all of the CpG cytosine residues in the target sequences in the region of interest are methylated; and/or the second methylation states are methylation states in which none of the CpG cytosine residues in the target sequences in the region of interest are methylated.

Any of the competing probes can be associated with a detectable label to facilitate generation of signals associated with hybridization of the competing probes. In some embodiments, the first competing probes of the plurality of competing probe sets are directly or indirectly associated with a first detectable label corresponding to the first methylation state, and detecting the first signal comprises detecting the first detectable label. In some embodiments, the second competing probes of the plurality of competing probe sets are directly or indirectly associated with a second detectable label corresponding to the second methylation state, and detecting the second signal comprises detecting the second detectable label. In some embodiments, the third competing probes of the plurality of competing probe sets are directly or indirectly associated with third detectable labels corresponding to third methylation states, and detecting the third signals comprises detecting the third detectable labels. In some embodiments, the further (e.g. fourth, fifth, sixth, etc.) competing probes of the plurality of competing probe sets are directly or indirectly associated with further detectable labels corresponding to further methylation states, and detecting the further signals comprises detecting the further detectable labels. In some embodiments, one or more competing probes of the plurality of competing probe sets are directly associated with the detectable labels. In some embodiments, one or more competing probes of the plurality of competing probe sets are configured to directly or indirectly bind and/or hybridize to detectably labeled probes comprising the detectable labels. The detectable labels directly or indirectly associated with the competing probes can be any suitable detectable labels. In some embodiments, the detectable labels are fluorophores. In some embodiments a detectable label directly or indirectly associated with a competing probe can be a combination of detectable labels.

In some embodiments, one or more competing probes of the plurality of competing probe sets are independently selected from the group consisting of: a probe not comprising an overhang, a probe comprising a 3′ and/or 5′ overhang; a circular probe; and a circularizable probe or probe set. In some embodiments, one or more competing probes of the plurality of competing probe sets comprise a 3′ overhang. In some embodiments, one or more competing probes of the plurality of competing probe sets comprises a barcode region associated with: a) the region of interest, b) the target sequence in the region of interest corresponding to the converted DNA target sequence to which it hybridizes, and/or c) the converted DNA target sequence to which it hybridizes. In some embodiments, the barcode region can be used to determine the identity of the region of interest (e.g. if different regions of interest corresponding to different genomic coordinates are being analyzed in the biological sample). In some embodiments, the barcode region can be used to determine the spatially localized position of the region of interest in the biological sample. In some embodiments, the barcode region can be used to generate any of the signals. For example, the barcode region can comprise a sequence that corresponds to the same methylation state that the competing probe corresponds to, and the sequence can be hybridized by an oligonucleotide that is directly or indirectly associated with a detectable label corresponding to the methylation state. Thus, it can be seen that a single competing probe can comprise a barcode region enabling identification and localization of the region of interest, as well as methylation analysis.

In some embodiments, the first signal, second signal, third signal, and/or further signals, are amplified. In some embodiments, the signal amplification comprises using one or more of the competing probes of the plurality of competing probe sets to perform: rolling circle amplification (RCA); hybridization chain reaction (HCR); linear oligonucleotide hybridization chain reaction (LO-HCR); primer exchange reaction (PER); assembly of branched structures; hybridization of a plurality of detectable probes directly or indirectly on the competing probes or products thereof; or any combination thereof.

In some embodiments, the first signal is collectively generated from the first competing probes of the plurality of competing probe sets that hybridize to the converted DNA. In some embodiments, the second signal is collectively generated from the second competing probes of the plurality of competing probe sets that hybridize to the converted DNA. In some embodiments, the third signal is collectively generated from the third competing probes of the plurality of competing probe sets that hybridize to the converted DNA. In some embodiments, the further signals are collectively generated from further competing probes of the plurality of competing probe sets that hybridize to the converted DNA.

This disclosure makes reference to corresponding components used in the described methods. It can be seen that various components of the method have a clear correspondence to one another. For example, in some embodiments, each competing probe set (and each competing probe therein) corresponds to a target sequence in the region of interest. In some embodiments, each competing probe of a competing probe set corresponds to a methylation state (which the converted DNA target sequence to which the competing probe is complementary is indicative of). In some embodiments, each first signal, second signal, third signal, and/or further signal corresponds to a methylation state (e.g. first, second, third, and/or further methylation state). In some embodiments, each detectable label (e.g. first, second, third, and/or further detectable label) used to generate the first signal, second signal, third signal, and/or further signal corresponds to a methylation state (e.g. first, second, third, and/or further methylation state).

### C. Sequencing Primers for Analyzing Methylation Status

In some embodiments, provided herein is a method. In some embodiments, provided herein is a method for interrogating methylation of a region of interest in a deoxyribonucleic acid (DNA). In some embodiments, provided herein is a method for interrogating methylation of a region of interest in a deoxyribonucleic acid (DNA) using a plurality of sequencing primers. In some embodiments, provided herein is a method for interrogating the methylation status of a region of interest in a deoxyribonucleic acid (DNA) using a plurality of sequencing primers. In some embodiments, interrogating the methylation comprises contacting a biological sample with a plurality of sequencing primers. In some embodiments, interrogating the methylation comprises detecting one or more signals. In some aspects, interrogating the methylation does not need to comprise downstream analysis of detected signals. In some embodiments, provided herein is a method for generating a signal in a biological sample. In some embodiments, provided herein is a method for detecting a signal in a biological sample. In some embodiments, provided herein is a method for analyzing the methylation status of a region of interest in a deoxyribonucleic acid (DNA) using a plurality of sequencing primers. In some embodiments, the method comprises providing a biological sample comprising converted DNA generated by converting the DNA, wherein the nucleotide sequence of the converted DNA is indicative of the methylation state of the DNA, and wherein the converted DNA comprises target residues indicative of the methylation states of corresponding cytosine residues in the region of interest. In some embodiments, the method comprises contacting the biological sample with a plurality of sequencing primers that hybridize to converted DNA target sequences that are adjacent and 3′ to target residues. In some embodiments, the method comprises contacting the biological sample with a plurality of sequencing primers that hybridize to converted DNA target sequences that are adjacent and 3′ (e.g. immediately 3′) to target residues indicative of unmethylated cytosines and/or methylated cytosines. In some embodiments, the method comprises contacting the biological sample with a plurality of sequencing primers that hybridize to converted DNA target sequences that are adjacent and 3′ to one or more of the target residues. For example, a sequencing primer can hybridize to a sequence that is adjacent and 3′ to a target residue, such that a sequence in the converted DNA comprising the target residue is configured to serve as template for extending the 3′ end of the sequencing primer (e.g. in a single base-pair or multiple base-pair extension reaction). In some embodiments, a sequencing primer can hybridize to a sequence that is immediately 3′ to a target residue, such that the target residue is configured to serve as template for a single-base extension reaction. In some embodiments, the method comprises performing an extension reaction (e.g. a single-base extension reaction) that incorporates a first detectably labeled nucleotide into sequencing primers that hybridize 3′ to target residues (e.g. one or more of the target residues) indicative of unmethylated cytosines. In some embodiments, the method comprises performing an extension reaction (e.g. a single-base extension reaction) that incorporates a second detectably labeled nucleotide into sequencing primers that hybridize 3′ to target residues (e.g. one or more of the target residues) indicative of methylated cytosines. In some embodiments, the extension reaction is a single-base extension reaction. In some embodiments, the extension reaction is a reaction that incorporates more than one base (e.g. incorporates 2, 3, 4, 5, or more bases).

In some embodiments, the method comprises detecting a first signal associated with incorporation of the first detectably labeled nucleotide. In some embodiments, the method comprises detecting a second signal associated with the second detectably labeled nucleotide. In some embodiments, the method comprises using the first signal and/or second signal to analyze the methylation status of the region of interest.

In some embodiments, decreased methylation in the region of interest (e.g. decreased proportion of methylated CpG cytosine residues) will result in a larger number of first detectably labeled nucleotides being incorporated into the sequencing primers during the extension reaction. Thus, in some embodiments, increased strength of the first signal generated from the first detectably labeled nucleotides that are incorporated in the extension reaction is indicative of increased proportion of unmethylated CpG cytosine residues. Similarly, increased methylation in the region of interest (e.g. increased proportion of methylated CpG cytosine residues) will result in a larger number of second detectably labeled nucleotides being incorporated into the sequencing primers during the extension reaction. Thus, in some embodiments, increased strength of the second signal generated from the second detectably labeled nucleotides that are incorporated in the extension reaction is indicative of increased proportion of methylated CpG cytosine residues. It can be seen that analysis of the first signal alone, or the second signal alone, can be sufficient to allow analysis of the methylation status of the region of interest. In some embodiments, the method comprises using the first signal and/or second signal to analyze the methylation status of the region of interest.

In some embodiments, the first signal corresponds collectively to the target residues indicative of unmethylated cytosine at the plurality of cytosine residues in the region of interest; and/or the second signal corresponds collectively to the target residues indicative of methylated cytosine at the plurality of cytosine residues in the region of interest. In some embodiments, the first signal corresponds collectively to at least 10, 20, 50, or 100 target residues indicative of unmethylated cytosine at the plurality of cytosine residues in the region of interest; and/or the second signal corresponds collectively to at least 10, 20, 50, or 100 target residues indicative of methylated cytosine at the plurality of cytosine residues in the region of interest. In some embodiments, the first signal is collectively generated from the first detectably labeled nucleotides that are incorporated in the extension reaction. In some embodiments, the second signal is collectively generated from the second detectably labeled nucleotides that are incorporated in the extension reaction. In some aspects, the collective generation of the first signal and second signal based on the methylation states of a plurality of cytosine residues across the region of interest allows the strength of the first signal and second signal to correspond to the methylation status of the region of interest as a whole. As in other methods described herein, the collective generation of signal(s) corresponding to many individual methylation states across a region of interest provides a mechanism for generating signals that are robust to individual non-specific detection events. In some embodiments, the plurality of sequencing primers comprise only sequencing primers targeting converted DNA target sequences adjacent to the target residues indicative of the methylation states of corresponding cytosine residues in the region of interest. In some embodiments, each sequencing primer of the plurality of sequencing primers targets a converted DNA target sequence adjacent to a target residue indicative of the methylation state of a corresponding cytosine residues in the region of interest. In some embodiments, the method does not comprise contacting the biological sample with sequencing primers that target converted DNA corresponding to regions of the DNA outside of the region of interest.

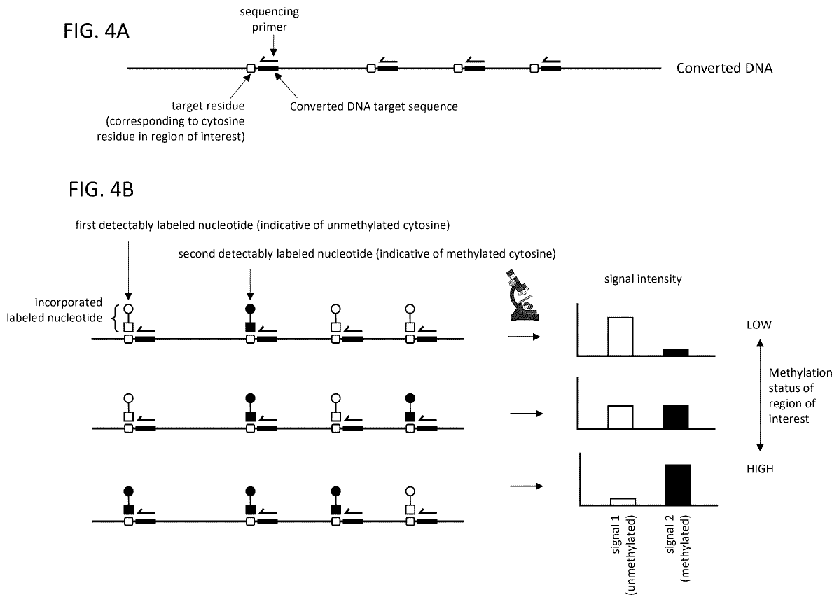

An exemplary embodiment of a method that utilizes sequencing probes for analyzing the methylation status of a region of interest is illustrated in FIGS. 4A-4B. FIG. 4A shows converted DNA generated from a region of interest of DNA in a biological sample. The converted DNA comprises a plurality of target residues corresponding to cytosine residues in the region of interest. The biological sample is contacted with sequencing primers that hybridize to converted DNA target sequences that are adjacent and 3′ to target residues (e.g. one or more of the target residues). FIG. 4B shows the result of performing an extension reaction (e.g. single-base extension reaction) that a) incorporates a first detectably labeled nucleotide into sequencing primers that hybridize 3′ to target residues indicative of unmethylated cytosines and/or b) incorporates a second detectably labeled nucleotide into sequencing primers that hybridize 3′ to target residues indicative of methylated cytosines. FIG. 4B shows three different scenarios ranging from low methylation status (top); to high methylation status (bottom). In the low methylation status scenario (top), a greater number of target residues correspond to unmethylated cytosines than methylated cytosines; consequently a greater number of first detectably labeled nucleotides are incorporated in the extension reaction than second detectably labeled nucleotides. The first signal collectively generated from the incorporated first detectably labeled nucleotides is relatively strong (e.g. in comparison to a reference signal or the second signal). In the high methylation status scenario, a relatively small proportion of target residues correspond to unmethylated cytosines, and a relatively large proportion of target residues correspond to methylated cytosines; consequently a greater number of second detectably labeled nucleotides are incorporated in the extension reaction, and the second signal is relatively strong (e.g. in comparison to a reference signal or the first signal). As described herein, either the first signal or second signal alone, or both the first signal and second signal can be used to analyze the methylation status of the region of interest.

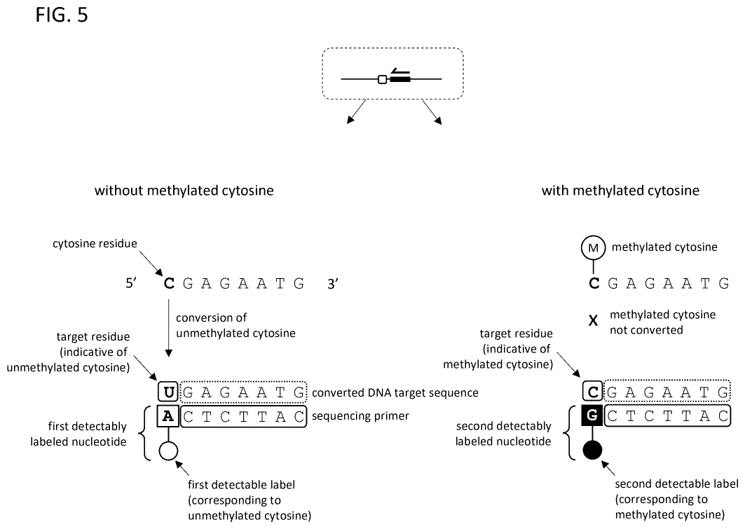

FIG. 5 shows schematics illustrating an exemplary single base-pair extension reaction for an individual sequencing primer. The reaction incorporates a detectably labeled nucleotide that corresponds to the methylation state of a cytosine in a region of interest that is unmethylated (left) or methylated (right). For purposes of illustration, in this example, the converted DNA is generated by converting unmethylated cytosines to uracil, and not converting methylated cytosines (e.g. by bisulfite sequencing). A sequencing primer is hybridized to a converted DNA target sequence that is immediately 3′ to a target residue corresponding to the cytosine residue. When the cytosine residue in the DNA is unmethylated (left), a first detectably labeled nucleotide (adenine) is complementary to the target residue (uracil) in the converted DNA, and the first detectably labeled nucleotide is thus incorporated into the sequencing primer in the single-base extension reaction. When the cytosine residue in the DNA is methylated (right), a second detectably labeled nucleotide (guanine) is complementary to the target residue (cytosine) in the converted DNA, and the second detectably labeled nucleotide is thus incorporated into the sequencing primer in the single-base extension reaction.

In some aspects, provided herein is a method of interrogating and/or analyzing a methylation status of a region of interest in a deoxyribonucleic acid (DNA). In some embodiments, the method comprises providing a biological sample comprising converted DNA generated by converting the DNA. In some embodiments, the nucleotide sequence of the converted DNA is indicative of the methylation state of the DNA. In some embodiments, the converted DNA comprises target residues indicative of the methylation state of corresponding cytosine residues in the region of interest. In some embodiments, the method comprises contacting the biological sample with one or more sequencing primers that hybridize to converted DNA target sequences in the converted DNA. In some embodiments, the method comprises performing a sequencing reaction to generate a first signal associated with target residues indicative of unmethylated cytosine at a plurality of cytosine residues in the region of interest. In some embodiments, the method comprises performing a sequencing reaction to generate a second signal associated with target residues indicative of methylated cytosine at a plurality of cytosine residues in the region of interest.

The sequencing reaction can be any suitable sequencing reaction. In some aspects, once the sequencing primers are hybridized, any suitable sequencing reaction can be used to generate the first signal and/or second signal. Any suitable and compatible sequencing reaction may be used, such as any described herein, including sequencing by synthesis (SBS), sequencing by binding (SBB), or sequencing by ligation (SBL), for example as described in Section III and elsewhere. In some embodiments, the sequencing reaction comprises sequencing by synthesis. In some embodiments, the sequencing reaction comprises sequencing by binding. In some embodiments, the sequencing reaction comprises sequencing by ligation. In some embodiments, the sequencing reaction comprises generating a product from the one or more sequencing primers. In some embodiments, the sequencing reaction is a single-base extension reaction.

In some embodiments, the first signal corresponds collectively to the target residues indicative of unmethylated cytosine at the plurality of cytosine residues in the region of interest; and/or the second signal corresponds collectively to the target residues indicative of methylated cytosine at the plurality of cytosine residues in the region of interest. As in other methods described herein, an advantage of this method is that the first signal and/or second signal can be generated collectively, and can be used to analyze the overall methylation status at the level of the entire region of interest, rather than at single-nucleotide resolution. This collective signal generation is robust to error at the single-nucleotide level.

In some aspects, provided herein is a method for interrogating and/or analyzing a methylation status of a region of interest in a deoxyribonucleic acid (DNA). In some embodiments, the method comprises providing a biological sample comprising converted DNA generated by converting the DNA. In some embodiments, the nucleotide sequence of the converted DNA is indicative of the methylation state of the DNA. In some embodiments, the converted DNA comprises target residues indicative of the methylation state of corresponding cytosine residues in the region of interest. In some embodiments, the method comprises contacting the biological sample with a plurality of sequencing primers that hybridize to converted DNA target sequences that are adjacent and 3′ to target residues (e.g. one or more of the target residues). In some embodiments, the method comprises performing an extension reaction that incorporates a first detectably labeled nucleotide into sequencing primers that hybridize 3′ to target residues indicative of unmethylated cytosines. In some embodiments, the method comprises performing an extension reaction that incorporates a second detectably labeled nucleotide into sequencing primers that hybridize 3′ to target residues indicative of methylated cytosines. In some embodiments, the method comprises performing an extension reaction that a) incorporates a first detectably labeled nucleotide into sequencing primers that hybridize 3′ to target residues indicative of unmethylated cytosines and b) incorporates a second detectably labeled nucleotide into sequencing primers that hybridize 3′ to target residues indicative of methylated cytosines. In some embodiments, the method comprises detecting a first signal associated with incorporation of the first detectably labeled nucleotide. In some embodiments, the method comprises detecting a second signal associated with the second detectably labeled nucleotide. In some embodiments, the method comprises using the first signal to analyze the methylation status of the region of interest. In some embodiments, the method comprises using the second signal to analyze the methylation status of the region of interest. In some embodiments, the method comprises using the first signal and the second signal to analyze the methylation status of the region of interest.

In some embodiments, the sequencing primers hybridize to converted DNA target sequences that are immediately 3′ to one or more of the target residues. In some embodiments, the sequencing primers hybridize to converted DNA target sequences that are immediately 3′ to the target residues indicative of unmethylated cytosines and/or methylated cytosines. In some embodiments, the extension reaction is a single-base extension reaction. In some embodiments, the first signal corresponds collectively to the target residues indicative of unmethylated cytosine at the plurality of cytosine residues in the region of interest. In some embodiments, the second signal corresponds collectively to the target residues indicative of methylated cytosine at the plurality of cytosine residues in the region of interest.

In some embodiments, the first signal corresponds collectively to at least 10, 20, 50, or 100 target residues indicative of unmethylated cytosine at the plurality of cytosine residues in the region of interest. In some embodiments, the second signal corresponds collectively to at least 10, 20, 50, or 100 target residues indicative of methylated cytosine at the plurality of cytosine residues in the region of interest. In some embodiments, the first signal corresponds collectively to at most 10, 20, 50, or 100 target residues indicative of unmethylated cytosine at the plurality of cytosine residues in the region of interest. In some embodiments, the second signal corresponds collectively to at most 10, 20, 50, or 100 target residues indicative of methylated cytosine at the plurality of cytosine residues in the region of interest. In some embodiments, the first signal is collectively generated from the first detectably labeled nucleotides that are incorporated in the extension reaction. In some embodiments, the second signal is collectively generated from the second detectably labeled nucleotides that are incorporated in the extension reaction.

Any suitable method can be used to detect the first signal and/or second signal. The detectably labeled nucleotides can comprise any suitable detectable labels. In some embodiments, the first detectably labeled nucleotide comprises a first detectable label and the second detectably labeled nucleotide comprises a second detectable label. The first and second detectable labels can allow for generation of the first and second signals, respectively. In some embodiments, the first and second detectably labeled nucleotides comprise first and second fluorophores that can be directly detected. In some embodiments, a detectably labeled nucleotide can comprise a detectable label that is not fluorescent, but that is detected by specific binding of a probe (e.g. antibody) that in turn is associated with a detectable label, such as a fluorophore.

The region of interest can be any suitable length. In some embodiments, the region of interest is at least 200 bases in length. In some embodiments, the region of interest is at least 500 bases in length. In some embodiments, the region of interest is at least 1000 bases in length. Any suitable number of sequencing primers can be used. For example, in some embodiments, the method comprises contacting the biological sample with at least 10, 20, 50, or 100 sequencing primers.

In any of the methods provided herein, any suitable method for converting the DNA can be used in connection with the methods described herein, such as bisulfite conversion or enzymatic conversion. In some embodiments, a non-cytosine target residue is indicative of unmethylated cytosine at the corresponding cytosine residue in the region of interest. In some embodiments, a cytosine target residue is indicative of methylated cytosine at the corresponding cytosine residue in the region of interest. In some embodiments, a non-cytosine target residue is indicative of methylated cytosine at the corresponding cytosine residue in the region of interest. In some embodiments, a cytosine target residue is indicative of unmethylated cytosine at the corresponding cytosine residue in the region of interest. In some embodiments, the non-cytosine target residue is uracil or dihydrouracil.

In some embodiments, the method comprises measuring the first signal, the second signal, and/or a reference signal. In some embodiments, interrogating and/or analyzing the methylation status of the region of interest comprises measuring the first signal, the second signal, and/or a reference signal. In some embodiments, interrogating and/or analyzing the methylation status of the region of interest comprises measuring the first signal. In some embodiments, interrogating and/or analyzing the methylation status of the region of interest comprises measuring the second signal. In some embodiments, interrogating and/or analyzing the methylation status of the region of interest comprises measuring a reference signal. In some embodiments, interrogating and/or analyzing the methylation status of the region of interest comprises measuring size, intensity, and/or abundance of the first signal, the second signal, and/or a reference signal.

In some embodiments, the method comprises comparing the first signal and/or second signal to the reference signal. In some embodiments, interrogating and/or analyzing the methylation status of the region of interest in the DNA comprises comparing the first signal and/or second signal to the reference signal. In some embodiments, interrogating and/or analyzing the methylation status of the region of interest in the DNA comprises comparing the first signal to the reference signal. In some embodiments, interrogating and/or analyzing the methylation status of the region of interest in the DNA comprises comparing the second signal to the reference signal. In some embodiments, interrogating and/or analyzing the methylation status of the region of interest in the DNA comprises comparing the first signal to the second signal. In some embodiments, increased size, intensity, and/or abundance of the first signal is indicative of the region of interest having a methylation status with a decreased proportion of cytosines that are methylated. In some embodiments, increased size, intensity, and/or abundance of the first signal in comparison to the reference signal is indicative of the region of interest having a methylation status with a decreased proportion of cytosines that are methylated. In some embodiments, increased size, intensity, and/or abundance of the second signal is indicative of the region of interest having a methylation status with an increased proportion of cytosines that are methylated. In some embodiments, increased size, intensity, and/or abundance of the second signal in comparison to the reference signal is indicative of the region of interest having a methylation status with an increased proportion of cytosines that are methylated. In some embodiments, interrogating and/or analyzing the methylation status of the region of interest in the DNA comprises comparing the first signal to the second signal. In some embodiments, increased size, intensity, and/or abundance of the first signal in comparison to the second signal is indicative of the region of interest having a methylation status with a decreased proportion of cytosines that are methylated. In some embodiments, the first and/or second signal is amplified.

In some embodiments, one or more of the sequencing primers are independently selected from the group consisting of: a probe not comprising an overhang, a probe comprising a 5′ overhang; and a circularizable probe or probe set. In some embodiments, one or more of the sequencing primers comprise a barcode region associated with: a) the region of interest, b) the target residue to which the sequencing primer hybridizes immediately 3′ to, and/or c) the converted DNA target sequence that the sequencing primer hybridizes to. In some embodiments, the barcode region can be used to determine the identity of the region of interest (e.g. if different regions of interest corresponding to different genomic coordinates are being analyzed in the biological sample). In some embodiments, the barcode region can be used to determine the spatially localized position of the region of interest in the biological sample. In some embodiments, the barcode region can be used to identify the converted DNA target sequence that the sequencing primer hybridizes to.

D. Methylation-dependent DNA conversion

In some aspects, the methods provided herein can comprise providing a biological sample comprising converted DNA generated by converting the DNA. In some embodiments, the method comprises converting the DNA. The DNA can be any suitable DNA. In some embodiments, the DNA is genomic DNA. In some embodiments, converting the DNA can comprise any suitable method for generating converted DNA, wherein the sequence of the converted DNA is indicative of the methylation state of the DNA. For example, converting the DNA can be methylation-state-dependent. In some embodiments, converting the DNA comprises converting cytosines in the DNA to a different nucleotide, wherein converting cytosines in the DNA is methylation-state-dependent. Any suitable method for converting the DNA may be used in connection with the methods described herein, including various methods of bisulfite conversion or enzymatic conversion that are described in detail elsewhere.

In some aspects, the methods provided herein comprise methylation-dependent nucleotide conversion (also referred to herein as DNA conversion or converting the DNA). In some embodiments, methylation-dependent nucleotide conversion comprises converting one or more nucleotides in DNA to a different nucleotide, wherein the converting depends on the methylation status of the nucleotide. For example, in some embodiments, a nucleotide is converted if the nucleotide is not methylated, and a nucleotide is not converted if the nucleotide is methylated. In some embodiments, the nucleotide is cytosine. In some embodiments, the nucleotide is converted if the nucleotide is unmethylated cytosine, and the nucleotide is not converted if the nucleotide is methylated cytosine (i.e., methylcytosine) or hydroxymethylcytosine. In some embodiments, the nucleotide is converted to uracil. In some embodiments, methylation-dependent nucleotide conversion comprises converting the DNA to single-stranded DNA, and/or DNA fragmentation. Thus, in some embodiments, the region of interest that is interrogated does not necessarily comprise a single contiguous DNA molecule.

In some aspects, methylation-dependent nucleotide conversion results in a sequence of DNA that corresponds to and is indicative of the methylation state of the DNA. In some aspects, a region of interest of DNA is interrogated to determine the methylation status of the region of interest, such as by any of the methods provided herein.

Any suitable method for methylation-dependent nucleotide conversion may be used with the methods disclosed herein. In some embodiments, methylation-dependent nucleotide conversion comprises bisulfite conversion. In some embodiments, bisulfite conversion comprises contacting the sample with a bisulfite reagent, such as sodium bisulfite or ammonium bisulfite. In some embodiments, contacting the sample with the bisulfite reagent leads to deamination of unmethylated cytosine to produce uracil, and does not lead to conversion of methylcytosine to uracil. Suitable kits and reagents for bisulfite conversion are commercially available, such as EpiMark® Bisulfite Conversion Kit (NEB Cat #: E3318S), and EZ DNA Methylation-Lightning Kit (Zymo Research Cat #: D5030). In some embodiments, bisulfite conversion converts DNA to single-stranded DNA. In some embodiments, bisulfite conversion leads to fragmentation of DNA. Thus, in some embodiments, the region of interest that is interrogated does not comprise a single contiguous DNA molecule. In some embodiments, the region of interest comprises a contiguous DNA molecule (e.g. a region of a chromosome), but the converted DNA corresponding to the region of interest does not (e.g. as a result of DNA conversion).

In some embodiments, DNA conversion comprises enzymatic conversion. In an exemplary enzymatic conversion, the DNA is first contacted with a TET enzyme (e.g., TET2) and an oxidation enhancer, such that 5-methylcytosine (5mC) is converted to 5-carboxycytosine (5caC), and 5-hydroxymethylcytosine (5-hmC) is converted to beta-glucosyl-5-hydroxymethylcytosine (5ghmC). 5caC and 5ghmC are protected from deamination in a subsequent enzymatic step. The DNA is made single-stranded, for example using formamide or sodium hydroxide. Next, the DNA is treated with the cytidine deaminase APOBEC, which deaminates unmethylated cytosines to uracil, but does not convert 5caC or 5ghmC. The result is that unmethylated cytosine in the DNA prior to treatment is converted to uracil, whereas methylcytosine and hydroxymethylcytosine in the DNA prior to treatment are not converted to uracil. A uracil in the treated DNA is indicative of an unmethylated cytosine in the DNA prior to treatment, whereas a cytosine in the treated DNA is indicative of methylcytosine or hydroxymethylcytosine in the DNA prior to treatment. In some embodiments, enzymatic conversion leads to reduced DNA damage, and/or greater sensitivity or otherwise improved detection of methylation status, in comparison to bisulfite conversion. Suitable kits for enzymatic methylation-dependent nucleotide conversion are commercially available, such as the NEBNext® Enzymatic Methyl-seq Kit (NEB Cat #: E7120S).

In some aspects, the methods provided herein comprise providing a biological sample comprising converted DNA. In some embodiments, the methods provided herein comprise converting the DNA. Any suitable method of converting DNA can be used in connection with the methods provided herein, such as bisulfite conversion or enzymatic conversion, which have been described elsewhere. In some aspects, a critical aspect of the conversion is that following conversion, the sequence of the converted DNA is indicative of and/or corresponds to the methylation state of the DNA.

In some embodiments, converting the DNA comprises converting cytosines in the DNA to a different nucleotide. In some embodiments, converting cytosines in the DNA is methylation-state-dependent. In some embodiments, cytosines that are not methylated are converted to a different nucleotide. In some embodiments, cytosines that are not methylated are converted to uracil. In some embodiments, cytosines that are methylated are converted to a different nucleotide. In some embodiments, cytosines that are methylated are converted to dihydrouracil. In some embodiments, cytosines that are methylated comprise methylated and/or hydroxymethylated cytosines. In some embodiments, converting the DNA comprises bisulfite conversion. In some embodiments, the bisulfite conversion comprises contacting the sample with a bisulfite reagent. In some embodiments, the bisulfite reagent is sodium bisulfite or ammonium bisulfite. In some embodiments, converting the DNA comprises enzymatic conversion.

In some embodiments, converting the DNA comprises converting a first nucleotide in the DNA to a different nucleotide, wherein the converting depends on the methylation state of the first nucleotide. In some embodiments, the first nucleotide is an unmethylated cytosine residue and the different nucleotide is uracil. In some embodiments, converting the unmethylated cytosine residue in the DNA to uracil comprises contacting the sample with a cytidine or cytosine deaminase. In some embodiments, the cytidine or cytosine deaminase converts unmethylated cytosine residues and methylcytosine residues to uracil. In some embodiments, prior to contacting the sample with the cytidine or cytosine deaminase, the method comprises contacting the sample with a methylcytosine dioxygenase enzyme that catalyzes the conversion of methylcytosine residues to hydroxymethylcytosine residues. In some embodiments, hydroxymethylcytosine residues are not converted to uracil. In some embodiments, converting unmethylated cytosine residues in the DNA to uracil comprises performing bisulfite conversion. In some embodiments, methylcytosine and/or hydroxymethylcytosine residues are not converted to uracil. In some embodiments, performing bisulfite conversion comprises contacting the sample with a bisulfite reagent. In some embodiments, the bisulfite reagent is sodium bisulfite or ammonium bisulfite. In some embodiments, contacting the sample with the bisulfite reagent leads to deamination of unmethylated cytosine to produce uracil, and does not lead to conversion of methylcytosine to uracil. In some embodiments, the first nucleotide is a methylcytosine or hydroxymethylcytosine residue and the different nucleotide is dihyrdrouracil (DHU), and wherein unmethylated cytostine residues in the DNA are not converted to uracil. In some embodiments, converting the first nucleotide to the different nucleotide comprises converting methylcytosine and/or hydroxymethylcytosine residues in the DNA to dihyrdrouracil by oxidation using ten-eleven translocation (TET) enzyme to 5-carboxylcytosine (5caC) prior to reduction to DHU.

### E. Analysis of Methylation Status in a Region of Interest

In some embodiments, the methods described herein comprise interrogating and/or analyzing the methylation status of a region of interest in a deoxyribonucleic acid (DNA). In some embodiments, the DNA is genomic DNA. In some embodiments, the method comprises converting the DNA to generate the converted DNA. The majority of methylation in human genomes occurs at cytosines at CpG sites (i.e. CpG cytosines). A CpG cytosine, as used herein, may refer to a cytosine of a CpG dinucleotide (a site where a cytosine nucleotide is followed by a guanine nucleotide in the 5′ to 3′ direction). However, non-CpG cytosines can be methylated in human genomes in some contexts (for example, see Moore, L. D. et al., Neuropsychopharmacology. 2013. 38 (1): 23-38, the content of which is herein incorporated by reference in its entirety). Further, methylation may occur in different contexts in the genomes of different species. Accordingly, the methods described herein may be used to interrogate methylation in any suitable context, including at CpG and/or non-CpG cytosines, and in human or non-human cells. In some embodiments, CpG cytosines are interrogated.

The terms “methylation state” and “methylation status” are used throughout this disclosure to refer to different aspects of methylation. As used herein, a methylation state can refer to a specific and/or defined state of methylation in a nucleotide or sequence of nucleotides. For example, a methylation state of a cytosine residue in DNA can be methylated or unmethylated. A methylation state of a target sequence can refer to a specific pattern of methylated and unmethylated cytosines in the target sequence. A methylation state of a target sequence can also be the proportion or percentage of cytosines or CpG cytosines in the target sequence that are methylated. Thus, many different target sequences can be said to have the same methylation states (e.g. if none of the CpG cytosines are methylated in each of the many different target sequences). In contrast, a target sequence having none of the CpG cytosines methylated and a target sequence having all of the CpG cytosines methylated have different methylation states. As used herein, the methylation status of a region of interest can refer to overall methylation in the region of interest as assessed using the methods herein. In some aspects, methylation status does not need to refer to an exact proportion of cytosines that are methylated, or to a specific pattern or sequence of methylation across the region of interest. In some aspects, a methylation status can be analyzed in a relative manner, for example by analyzing collectively generated signals corresponding to methylation states. In some aspects, a methylation status of a region of interest is reflected by an overall measure of the level of methylation across the entire region of interest, e.g. by analyzing collectively generated signals as described herein.

A methylation state (e.g. a methylation state, first methylation, second methylation state, etc.) of a target sequence, as used herein, can be any suitable methylation state. For example, a methylation state can comprise a state of a target sequence in which none of the CpG cytosines in the target sequence are methylated. Two different target sequences can have different nucleotide sequences, but have the same methylation state, for example, if in each of the two different target sequence, none of the CpG cytosines are methylated. By extension, a plurality of different target sequences in a region of interest can have the same methylation state (e.g. a first methylation state in which none of the CpG cytosines are methylated, or a second methylation state in which all of the CpG cytosines are methylated). In some embodiments, a methylation state can comprise a state in which none of the CpG cytosines are methylated, or a state in which all of the CpG cytosines are methylated. A methylation state can also be a state in which any suitable proportion of CpG cytosines are methylated. For example, a methylation state can be a state in which none, one fourth, one third, one half, two thirds, three fourths, or all of the cytosines or CpG cytosines in a target sequence are methylated.

A methylation status of a region of interest, as used herein, can generally refer to the proportion of cytosines in the region of interest that are methylated. A high methylation status may refer to a methylation status in which a large proportion of cytosines in the region of interest (e.g. >50% of cytosines or CpG cytosines) are methylated. A low methylation status may refer to a methylation status in which a small proportion of cytosines in the region of interest (e.g. <50% of cytosines or CpG cytosines) are methylated. A methylation status does not need to correspond to an absolute value, and may be determined in a relative manner. For example, in some embodiments, a methylation status of a first region of interest can be analyzed in comparison to the methylation status of a second region of interest, such as a second region of interest in a different cell and corresponding to the same genetic locus as the first region of interest. In some embodiments, a methylation status of a first region of interest can be analyzed in comparison to the methylation status of a second region of interest in the same cell and corresponding to the same genetic locus as the first region of interest but on a different chromosome. In some embodiments, a methylation status of a first region of interest can be analyzed in comparison to the methylation status of a second region of interest in the same cell and corresponding to a different genetic locus from the first region of interest. Thus, the methods can be used to compare methylation status among a plurality of regions of interest.

In some embodiments, the methylation status of the region of interest is, corresponds to, and/or is indicative of, the proportion of cytosine residues that are methylated in the region of interest. In some embodiments, the methylation status of the region of interest is, corresponds to, and/or is indicative of, the proportion of CpG cytosine residues that are methylated in the region of interest.

In some embodiments, a methylation status is represented by a value corresponding to the methylation status of a region of interest as a whole. In some embodiments, the methylation status for a region of interest is a value corresponding to an aggregate, average, or overall readout of a methylation status at the region of interest. In some embodiments, the value is determined or estimated based on detected signals that correspond to different methylation states (e.g. of target sequences or cytosine residues) within the region of interest. In some embodiments, the value is calculated by comparing the intensities of signals corresponding to different methylation states in the region of interest. For example, in some embodiments, a first signal corresponding to 0% methylation is detected and a second signal corresponding to 100% methylation is detected, and the signal intensities are proportional to the number of interrogated target sequences within the region of interest with 0% or 100% methylation, respectively. In some embodiments, the value representing methylation status is a relative value. In some embodiments, the methylation status of a region of interest is defined in relation to one or more other regions of interest. For example, in some embodiments, at a first region of interest, the signal corresponding to 0% methylation has a higher intensity than the signal corresponding to 100% methylation, indicative of a relatively low methylation status; at a second region of interest, the signal corresponding to 100% methylation has a higher intensity than the signal corresponding to 0% methylation, indicative of a relatively high methylation status, and/or a methylation status that is higher than the first region of interest.

One or more regions of interest can be detected at one or more locations in a biological sample. For example, a region of interest in genomic DNA can be detected at two or more locations, corresponding to the region of interest in two different cells in the sample. In some embodiments, different regions of interest can be detected at different locations in a biological sample, in the same cell and/or in different cells. In some embodiments, when different regions of interest are interrogated, an identification step is performed, in which the locations of the different regions of interest are identified, for example, using multiplexed FISH assays or any suitable assay for detecting the locations of different nucleic acid sequences. In some embodiments, the identification step can be performed at any stage, including before, during, or after generation and detection of signals corresponding to methylation status. In some embodiments, a separate identification step is not performed. For example, in some embodiments, only one region of interest is interrogated, and the locations of the signals corresponding to methylation status of the region of interest are indicative of the location of the region of interest.

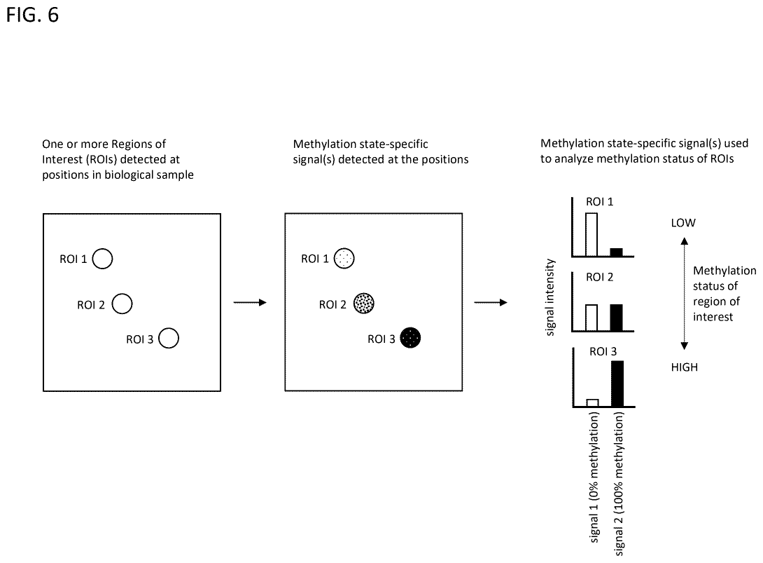

FIG. 6 shows schematics illustrating an exemplary workflow for in situ analysis of DNA methylation. One or more regions of interest are detected at one or more locations in a biological sample (left), for example using fluorescence in situ hybridization (FISH; e.g. multiplexed FISH) and fluorescence imaging. The locations of the regions of interest are recorded. Next, signals corresponding to different methylation states at multiple interrogated sites (e.g., target sequences or cytosine residues) within each region of interest are detected at the one or more locations (middle). For example, as shown in FIG. 6, a first signal is associated with 0% CpG cytosine methylation states and a second signal is associated with 100% CpG cytosine methylation states, and the intensity of each signal is proportional to the number of interrogated sites having the associated methylation state. In some embodiments, more than two signals, each associated with a different methylation state can generated, detected, and analyzed (e.g., 0%, 25%, 50%, 75%, and 100% CpG cytosine methylation states). The relative intensities of the signals can be compared to determine the relative methylation status of each region of interest as a whole (right). For example, as shown for a first region of interest (ROI 1), a first signal associated with 0% methylation states has a relatively high intensity and a second signal associated with 100% methylation states has a relatively low intensity, indicating that the region of interest has a relatively low methylation status (e.g. a low proportion of CpG cytosines in the first region of interest are methylated). As shown for a second region of interest (ROI 2), a first signal associated with 0% methylation states is relatively comparable in intensity to a second signal associated with 100% methylation states, indicating that the region of interest has a relatively intermediate methylation status. As shown for a third region of interest (ROI 3), a first signal associated with 0% methylation states has a relatively low intensity and a second signal associated with 100% methylation states has a relatively high intensity, indicating that the region of interest has a relatively high methylation status (e.g., a high proportion of CpG cytosines in the third region of interest are methylated). The generation and analysis of signals associated with methylation states as illustrated in FIG. 6 can be performed in accordance with any of the methods provided herein. The signals can be analyzed in any other suitable fashion, such as by analyzing a single methylation-state-specific signal, for example in comparison to a reference signal that is not a methylation-state-specific signal, as described herein.

In some embodiments, the methods comprise comparing a signal corresponding to a methylation status of a region of interest to a reference signal. The reference signal can be any suitable reference signal. In some embodiments, the reference signal is a control reference signal, such as one generated from a locus with a known methylation state. In some embodiments, the reference signal is a signal corresponding to the methylation status of the same region of interest in a different cell in the biological sample. In this way, the methylation status of the region of interest can be compared between two cells in the biological sample by detecting and analyzing signals generated using the same set of probes (e.g. methylations-state-specific probes, competing probes, or sequencing primers). Similarly, the methylation status of the region of interest can be profiled and/or compared among any number of cells in a biological sample. In some embodiments, the reference signal is generated from probes (e.g. methylation state-specific probes, competing probes, or sequencing primers) targeting converted DNA generated from the same region of interest in a different cell in the biological sample. Alternatively, the reference signal can be a signal that is generated in a manner that is independent from methylation status. In some embodiments, a reference signal is generated from detectably labeled (e.g., fluorescently labeled) probes that bind directly or indirectly to sequences in the converted DNA that do not correspond to cytosines exhibiting differential methylation. In some embodiments, a reference signal is a fluorescent DNA stain such as a DAPI stain. In some embodiments, a reference signal is generated via detection of non-nucleic acid molecules in the biological sample (e.g. proteins).

In some aspects, the methods provided herein for analyzing the methylation status of a region of interest are not exact, in that they do not provide methylation information at single-nucleotide resolution. Instead, the methods allow for the generation of signals that are generated collectively based on the methylation states of a plurality of target sequences and/or cytosine residues in a region of interest. The strength of a collectively generated methylation-state-specific signal can correspond to the overall methylation status of a region of interest. Analyzing methylation status at the level of a region of interest provides several advantages. For example, the analysis can be both more efficient and more informative than determining methylation status individually at single nucleotides or individual target sequences. Analysis at the level of a region of interest, as provided in the methods described herein, in some aspects is analogous to “binning” single-nucleotide methylation information, e.g. as determined from WGS-based methylation profiling, which is often performed to increase the efficiency and informativeness of analysis. Furthermore, the methods provide a readout of methylation status that is robust to error at the level of individual cytosine residues, target sequences, and/or probes that hybridize to converted DNA target sequences (e.g. methylation-state-specific probes or competing probes).

In some embodiments, the region of interest can comprises any suitable number of target sequences. For example, in some embodiments, the region of interest comprises 2, 3, 4, 5, 6, 7, 8, 9, 10, or more target sequence. In some embodiments, the region of interest comprises fewer than 20, at least 20, at least 50, at least 100, or at least 500 target sequences. In some embodiments, a target sequence is between about 6 and about 50, between about 6 and about 40, between about 6 and about 30, between about 6 and about 25, or between about 10 and about 20 nucleotides in length. In some embodiments, the region of interest is between about 100 bases and about 1 kilobase, between about 1 and about 15 kilobases, between about 15 and about 25 kilobases, between about 25 and about 50 kilobases, between about 50 and about 75 kilobases, between about 75 and about 100 kilobases, or more than 100 kilobases in length. In some embodiments, the region of interest is at least 50 bases in length. In some embodiments, the region of interest is at least 100 bases in length. In some embodiments, the region of interest is at least 200 bases in length. In some embodiments, the region of interest is at least 300 bases in length. In some embodiments, the region of interest is at least 400 bases in length. In some embodiments, the region of interest is at least 500 bases in length. In some embodiments, the region of interest is at least 1000 bases in length. The size of the region of interest may be any suitable size. For example, the region of interest may be between about 100 bases and 1 kilobase, between about 1 and about 15 kilobases, between about 15 and about 25 kilobases, between about 25 and about 50 kilobases, between about 50 and about 75 kilobases, between about 75 and about 100 kilobases, or more than 100 kilobases in length. The region of interest may comprise any number of target sequences, such as at least 20, at least 50, at least 100, at least 200, or at least 500 target sequences. Each (or one or more) target sequence may be independently between 10 and 50 nucleotides in length. In some embodiments, the region of interest is between about 100 bases and 1 kilobase, between about 1 kilobase and about 15 kilobases, between about 15 kilobases and about 25 kilobases, between about 25 kilobases and about 50 kilobases, between about 50 kilobases and about 75 kilobases, between about 75 kilobases and about 100 kilobases, or more than 100 kilobases in length. In some embodiments, the region of interest is at least 200 bases in length. In some embodiments, the region of interest is at least 500 bases in length. In some embodiments, the region of interest is at least 1000 bases in length.

In some embodiments, the method comprises identifying the spatially localized position of the region of interest in the biological sample simultaneously with, before, or after contacting the region with competing probes to determine methylation status. For example, multiplexed fluorescence in situ hybridization (FISH) can be used to identify the spatial coordinates of one or more regions of interest in the DNA in the biological sample that are to be analyzed for methylation status. In some embodiments, once the locations of the regions of interest are identified, the methylation status at each (or one or more) region of interest can be analyzed. In some aspects, the methods can comprise determining the spatially localized position of the region of interest in the biological sample. Any suitable method for identifying the spatially localized position of the region of interest may be used. In some aspects, determining the spatially localized position of the region of interest allows for analysis of methylation status of the region of interest in situ in the biological sample. In some embodiments, determining the spatially localized position of the region of interest can allow for a methylation-state-specific signal to be analyzed at the spatially localized position. In some aspects, by analyzing a signal only at the spatially localized position, the accuracy of the method may be increased (e.g. by disregarding potential background or non-specific signal at locations outside of the spatially localized position). In some aspects, determining the spatially localized position of a first region of interest can allow the region of interest to be differentiated from a second region of interest at a second spatially localized position, such as a second region of interest in the same cell and corresponding to a different genetic locus from the first region of interest. Identifying the spatially distinct positions of two different regions of interest can also allow the two regions of interest to be analyzed using a similar readout (e.g. using the same fluorophore to generate a methylation-state-specific signals for each region of interest).

The spatially localized position of the region of interest can be determined prior to and/or after converting the DNA. For example, the region of interest can be identified in the DNA prior to converting the DNA using probes that hybridize to the region of interest. Once the spatially localized position of the region of interest in the biological sample is identified and/or recorded, the DNA can be converted, and methylation status of the region of interest can be analyzed using the converted DNA according to any of the methods provided herein. Alternatively, the spatially localized position of the region of interest can be identified using probes that hybridize to the converted DNA at locations corresponding to the region of interest. In some embodiments, the probes used to identify the spatially localized position of the region of interest do not include the same probes as are used for analyzing methylation status. In some embodiments, the probes used to identify the spatially localized position of the region of interest can include the same probes as are used for analyzing methylation status (e.g. methylation-state-specific probes, competing probes, or sequencing probes). For example, in some embodiments, the probes used for analyzing methylation status (e.g. methylation-state-specific probes, competing probes, or sequencing probes) can include a barcode region associated with the region of interest and/or target sequence, which can be used to generate a signal that allows for the identification of the spatially localized position of the region of interest.

In some embodiments, the method comprises determining a spatially localized position of the region of interest in the biological sample. In some embodiments, the methylation status of the region of interest is analyzed at the spatially localized position of the region of interest in the biological sample. In some embodiments, the spatially localized position of the region of interest in the biological sample can be determined prior to and/or after generation of the converted DNA. In some embodiments, the spatially localized position of the region of interest is determined prior to generation of the converted DNA. For example, in some embodiments, determining the spatially localized position of the region of interest can comprise contacting the biological sample with one or more detectably-labeled probes that hybridize to the region of interest in the DNA and detecting a signal associated with the one or more detectably-labeled probes at the spatially localized position. In some embodiments, the spatially localized position of the region of interest is determined after generation of the converted DNA. For example, in some embodiments, determining the spatially localized position of the region of interest comprises contacting the sample with one or more detectably-labeled probes that hybridize to a converted region of interest resulting from conversion of the DNA of the region of interest and detecting a signal associated with the one or more detectably-labeled probes at the spatially localized position. In some embodiments, the one or more detectably-labeled probes comprise one or more of the competing probes; one or more of the methylation-state-specific probes; or one or more of the sequencing primers. In some embodiments, the one or more detectably-labeled probes do not comprise the competing probes; the methylation-state-specific probes; or the sequencing primers. In some embodiments, the spatially localized position of the region of interest is in a cell in a tissue. In some embodiments, the spatially localized position of the region of interest is in a nucleus in a cell in a tissue.

In some embodiments, the region of interest is a first region of interest, and the methylation status of a second region of interest is analyzed. In some embodiments, the first and second region of interest are at the same genomic locus on different chromosomes. In some embodiments, the first and second region of interest are at the same genomic locus in different cells. In some embodiments, the first and second region of interest are at different genomic loci.

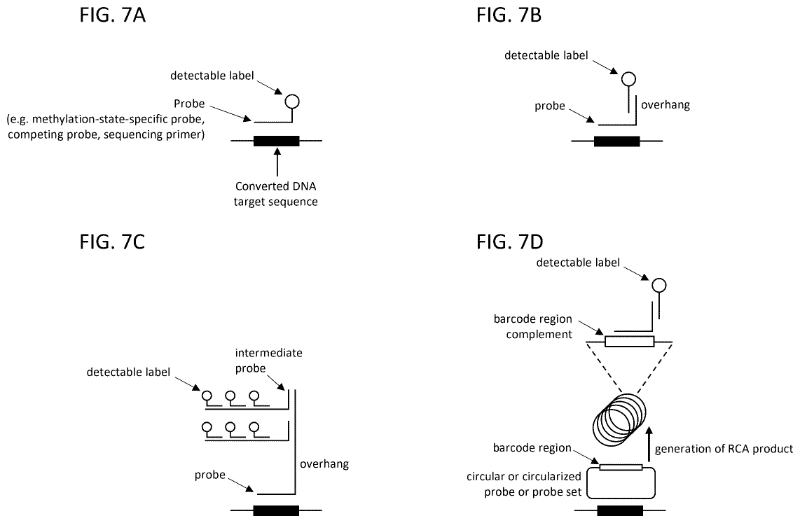

The probes that hybridize to converted DNA target sequences described herein (e.g. a methylation-state-specific probe, competing probe, or sequencing probe) can be detected by any suitable means. FIGS. 7A-7D show schematics illustrating exemplary arrangements of a detectable label associated with a probe (e.g. a methylation-state-specific probe, competing probe, or sequencing probe) that is hybridized to a converted DNA target sequence. In some aspects, any of the probes described herein, including any methylation-state-specific probe, any competing probe, or any sequencing probe, can be detected by any suitable method. In FIG. 7A, the probe is directly labeled (e.g., is conjugated to the detectable label). In FIG. 7B, the probe comprises an overhang sequence that hybridizes to a labeled probe (i.e., a detection oligonucleotide). FIG. 7C and FIG. 7D depict arrangements in which a single probe is associated with a plurality of the detectable label. In FIG. 7C, the probe comprises an overhang that is hybridized at multiple sites by intermediate probes. The intermediate probes in turn are hybridized by a plurality of detection oligonucleotides comprising the detectable label. In FIG. 7D, the hybridized probe is used for rolling circle amplification (RCA). In some embodiments, the probe is circular. In some embodiments, the probe comprises a circularizable probe (e.g., a padlock probe) or probe set, and is circularized following hybridization. The circular or circularized probe or probe set comprises a barcode region, and serves as a template for RCA, generating multiple copies of the complement of the barcode region, which is hybridized directly or indirectly by a detection oligonucleotide. Any of the probes herein (e.g. methylation-state-specific probes, competing probes, or sequencing probes) can comprise any suitable nucleic acid species (e.g., DNA, RNA, PNA, LNA).

In some embodiments, the biological sample is non-homogenized and optionally selected from the group consisting of a formalin-fixed, paraffin-embedded (FFPE) sample, a frozen tissue sample, and a fresh tissue sample. In some embodiments, the biological sample is fixed. In some embodiments, the biological sample is not fixed. In some embodiments, the biological sample is permeabilized. In some embodiments, the biological sample is embedded in a matrix, optionally wherein the matrix comprises a hydrogel. In some embodiments, the biological sample is cleared, optionally wherein the clearing comprises contacting the biological sample with a proteinase. In some embodiments, the biological sample is crosslinked. In some embodiments, the biological sample is or comprises a cell. In some embodiments, the biological sample is a tissue sample. In some embodiments, the biological sample is a tissue section. In some embodiments, the biological sample is a tissue slice. In some embodiments, the biological sample is a tissue slice between about 1 μm and about 50 μm in thickness, optionally wherein the tissue slice is between about 5 μm and about 35 μm in thickness.

In some aspects, any of the methods provided herein can comprise one or more wash steps. In some embodiments, the wash steps are performed at any suitable stage in the method. In some embodiments, one or more wash steps are performed following probe hybridization, for example to remove unbound probes.

### F. Kits, Systems, and Compositions for Analysis of Methylation Status

In some aspects, provided herein are kits, systems, and compositions. In some aspects, provided herein are kits, systems, and compositions for interrogating or analyzing methylation, for example in accordance with any of the methods provided herein. In some aspects, any of the kits, systems, or compositions described herein can comprise any component described in connection with another one of the kits, systems, or compositions provided herein. In some aspects, any of the kits, systems, or compositions described herein can comprise any component described in connection with the methods provided herein. Similarly, any of the methods provided herein can comprise the use of any component described in the kits, compositions, or systems provided herein. Such components include but are not limited to any of the biological samples, DNA, converted DNA, regions of interest, reagents for converting DNA, probes (e.g. methylation-state-specific probes), detectable labels, competing probes, competing probe sets, detectable labels, sequencing primers, detectably labeled nucleotides, detectably labeled probes, portions or sub-components of any of the foregoing, or combinations of any of the foregoing. In some aspects, any of the compositions provided herein can comprise a composition that is generated in the course of performing any of the methods provided herein.

In some aspects, provided herein are kits. The various components of the kit may be present in separate containers or certain compatible components may be pre-combined into a single container. In some embodiments, the kits further contain instructions for using the components of the kit to practice the provided methods. In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein. In some embodiments, the kits contain reagents for detection and/or sequencing, such as probes and detectable labels. In some embodiments, the kits optionally contain other components, for example primers.

In some aspects, provided herein are kits for interrogating a methylation status of a region of interest in a deoxyribonucleic acid (DNA) in a biological sample. In aspects, the kits can be used to analyze the methylation status of the region of interest. In some embodiments, the kits can be used to generate a signal corresponding to methylation in the region of interest in the biological sample, such as a methylation-state-specific signal.

In some aspects, provided herein is a kit. In some embodiments, the kit is for interrogating methylation of a region of interest in a deoxyribonucleic acid (DNA) in a biological sample. In some embodiments, the kit is for interrogating a methylation status of a region of interest in a deoxyribonucleic acid (DNA) in a biological sample. In some embodiments, the biological sample comprises converted DNA generated by converting the DNA. In some embodiments, the nucleotide sequence of the converted DNA is indicative of the methylation state of the DNA. In some embodiments, the kit comprises a plurality of methylation-state-specific probes. In some embodiments, each (or one or more) methylation-state-specific probe is complementary to a converted DNA target sequence indicative of a methylation state of a target sequence in the region of interest. In some embodiments, the plurality of methylation state-specific probes collectively target a plurality of converted DNA target sequences corresponding to a plurality of target sequences in the region of interest.

In some embodiments, the plurality of methylation state-specific probes are configured to be used to generate a methylation-state-specific signal associated with hybridization of methylation-state-specific probes to the converted DNA, for example in accordance with any of the methods provided herein that involve signal detection. In some embodiments, the plurality of methylation-state-specific probes is a plurality of first methylation-state-specific probes. In some embodiments, each first methylation-state-specific probe is complementary to a converted DNA target sequence indicative of a first methylation state of a target sequence in the region of interest. In some embodiments, the methylation-state-specific signal is a first methylation-state-specific signal. In some embodiments, the kit further comprises a plurality of second methylation-state-specific probes. In some embodiments, each second methylation-state-specific probe is complementary to a converted DNA target sequence indicative of a second methylation state of a target sequence in the region of interest. In some embodiments, the plurality of second methylation state-specific probes collectively target a plurality of converted DNA target sequences corresponding to a plurality of target sequences in the region of interest. In some embodiments, the second methylation state-specific probes are configured to be used to generate a second methylation-state-specific signal associated with hybridization of second methylation-state-specific probes to the converted DNA. In some embodiments, the plurality of converted DNA target sequences targeted by the first methylation state-specific probes and the plurality of converted DNA target sequences targeted by the second methylation state-specific probes correspond to the same plurality of target sequences in the region of interest. In some embodiments, the plurality of converted DNA target sequences targeted by the first methylation state-specific probes and the plurality of converted DNA target sequences targeted by the second methylation state-specific probes do not correspond to the same plurality of target sequences in the region of interest. In some embodiments, the plurality of methylation-state-specific probes comprises at least 3, 5, 10, 20, 50, 100, or 500 methylation-state-specific probes. In some embodiments, the plurality of first methylation-state-specific probes comprises at least 3, 5, 10, 20, 50, 100, or 500 first methylation-state-specific probes. In some embodiments, the plurality of second methylation-state-specific probes comprises at least 3, 5, 10, 20, 50, 100, or 500 second methylation-state-specific probes. In some embodiments, each target sequence in the region of interest comprises 1, 2, 3, 4, or more cytosine residues. In some embodiments, each target sequence in the region of interest comprises 1, 2, 3, 4, or more CpG cytosine residues. In some embodiments, the methylation-state-specific probes are directly or indirectly associated with a detectable label. In some embodiments, the first methylation-state-specific probes are directly or indirectly associated with a first detectable label. In some embodiments, the second methylation-state-specific probes are directly or indirectly associated with a second detectable label. In some embodiments, the kit comprises the detectable label. In some embodiments, the kit comprises the first detectable label. In some embodiments, kit comprises the second detectable label. In some embodiments, the kit comprises a reagent for converting the DNA.

The reagent for converting the DNA can be any suitable reagent, such as any provided herein. In some embodiments, the reagent comprises a bisulfite reagent. In some embodiments, the reagent comprises one or more enzymes. In some embodiments, the biological sample is any suitable biological sample, such as any provided herein. In some embodiments, the biological sample is a cell or a tissue sample. In some embodiments, the kit comprises the biological sample. In some embodiments, the kit does not comprise the biological sample.

In some aspects, provided herein is a kit for interrogating methylation of a region of interest in a deoxyribonucleic acid (DNA) in a biological sample. In some aspects, provided herein is a kit for interrogating a methylation status of a region of interest in a deoxyribonucleic acid (DNA) in a biological sample. In some embodiments, the biological sample comprises converted DNA generated by converting the DNA. In some embodiments, the nucleotide sequence of the converted DNA is indicative of the methylation state of the DNA. In some embodiments, the kit comprises a plurality of competing probe sets. In some embodiments, each (or one or more) competing probe set comprises: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest.

In some embodiments, the plurality of competing probe sets are configured to be used to generate a first signal associated with hybridization of first competing probes of the plurality of competing probe sets to the converted DNA. In some embodiments, the plurality of competing probe sets are configured to be used to generate a second signal associated with hybridization of second competing probes of the plurality of competing probe sets to the converted DNA. In some embodiments, one or more competing probe sets of the plurality of competing probe sets further comprise a third competing probe that is complementary to a third converted DNA target sequence indicative of a third methylation state of the target sequence in the region of interest. In some embodiments, the plurality of competing probe sets are configured to be used to generate a third signal associated with hybridization of third competing probes of the plurality of competing probe sets to the converted DNA. In some embodiments, one or more competing probe sets of the plurality of competing probe sets comprise further competing probes that are complementary to further converted DNA target sequences indicative of further methylation states of the target sequence in the region of interest. In some embodiments, the plurality of competing probe sets are configured to be used to generate further signals associated with hybridization of further competing probes of the plurality of competing probe sets to the converted DNA, for example according to any of the methods provided herein. In some embodiments, the first signal corresponds to the first methylation states of the target sequences. In some embodiments, the second signal corresponds to the second methylation states of the target sequences. In some embodiments, the third signal corresponds to the third methylation states of the target sequences. In some embodiments, the further signals correspond to further methylation states of the target sequences. In some embodiments, any of the competing probes are configured to be used to generate a signal, for example in accordance with any of the methods provided herein that involve signal detection.

In some embodiments, the plurality of competing probe sets comprises: a first competing probe set comprising competing probes complementary to converted DNA target sequences indicative of different methylation states of a first target sequence in the region of interest; and a second competing probe set comprising competing probes complementary to converted DNA target sequences indicative of different methylation states of a second target sequence in the region of interest. In some embodiments, the plurality of competing probe sets further comprises a third competing probe set comprising competing probes complementary to converted DNA target sequences indicative of different methylation states of a third target sequence in the region of interest. In some embodiments, the plurality of competing probe sets comprises further competing probe sets, each further competing probe set comprising competing probes complementary to converted DNA target sequences indicative of different methylation states of further target sequences in the region of interest. In some embodiments, the plurality of competing probe sets comprises at least 10, 20, 50, 100, or 500 competing probe sets for at least 10, 20, 50, 100, or 500 target sequences in the region of interest, respectively. In some embodiments, the plurality of competing probe sets comprises any suitable number of competing probe sets. In some embodiments, a competing probe set is provided for each (or one or more) of the target sequences in the region of interest.

In some embodiments, the first, second, third, and/or further methylation states of the target sequences in the region of interest each comprise a different proportion of methylated CpG cytosine residues. In some embodiments, each target sequence in the region of interest comprises 1, 2, 3, 4, or more cytosine residues, and/or each target sequence in the region of interest comprises 1, 2, 3, 4, or more CpG cytosine residues. In some embodiments, each (or one or more) target sequence in the region of interest is independently between 10 and 50 nucleotides in length. In some embodiments, the first methylation states of the target sequences in the region of interest comprise fewer methylated cytosines than the second methylation states of the target sequences in the region of interest. In some embodiments, the first methylation states of the target sequences in the region of interest comprise a smaller proportion of methylated CpG cytosine residues than the second methylation states of the target sequences in the region of interest. In some embodiments, the first methylation states are methylation states in which none of the CpG cytosine residues in the target sequences in the region of interest are methylated. In some embodiments, the second methylation states are methylation states in which all of the CpG cytosine residues in the target sequences in the region of interest are methylated. In some embodiments, the first methylation states of the target sequences in the region of interest comprise more methylated cytosines than the second methylation states of the target sequences in the region of interest. In some embodiments, the first methylation states of the target sequences in the region of interest comprise a larger proportion of methylated CpG cytosine residues than the second methylation states of the target sequences in the region of interest. In some embodiments, the first methylation states are methylation states in which all of the CpG cytosine residues in the target sequences in the region of interest are methylated. In some embodiments, the second methylation states are methylation states in which none of the CpG cytosine residues in the target sequences in the region of interest are methylated.

In some embodiments, the first competing probes of the plurality of competing probe sets are directly or indirectly associated with a first detectable label corresponding to the first methylation state. In some embodiments, the second competing probes of the plurality of competing probe sets are directly or indirectly associated with a second detectable label corresponding to the second methylation state. In some embodiments, the third and/or further competing probes of the plurality of competing probe sets are directly or indirectly associated with third and/or further detectable labels corresponding to third and/or further methylation states. In some embodiments, the kit comprises the first detectable label. In some embodiments, the kit comprises the second detectable label. In some embodiments, the kit comprises the third detectable label, and/or further detectable labels. In some embodiments, the first detectable label, second detectable label, third detectable label, and/or further detectable labels are optical labels, optionally fluorophores. In some embodiments, the kit comprises a reagent for converting the DNA, such as any reagent for converting the DNA provided herein. In some embodiments, the biological sample is any suitable biological sample. In some embodiments, the biological sample is a cell or a tissue sample.

In some aspects, provided herein is a kit for interrogating methylation of a region of interest in a deoxyribonucleic acid (DNA) in a biological sample. In some aspects, provided herein is a kit for interrogating a methylation status of a region of interest in a deoxyribonucleic acid (DNA) in a biological sample. In some embodiments, the biological sample comprises converted DNA generated by converting the DNA. In some embodiments, the nucleotide sequence of the converted DNA is indicative of the methylation state of the DNA. In some embodiments, the converted DNA comprises target residues indicative of the methylation state of corresponding cytosine residues in the region of interest. In some embodiments, the kit comprises a plurality of sequencing primers that hybridize to converted DNA target sequences that are adjacent and 3′ to target residues.

In some embodiments, the sequencing primers hybridize to converted DNA target sequences that are immediately 3′ to target residues. In some embodiments, the kit further comprises a first detectably labeled nucleotide that is configured to be incorporated into sequencing primers that hybridize 3′ to target residues indicative of unmethylated cytosines. In some embodiments, the kit comprises a second detectably labeled nucleotide that is configured to be incorporated into sequencing primers that hybridize 3′ to target residues indicative of methylated cytosines. In some embodiments, the first detectably labeled nucleotide is configured to be incorporated in a single-base extension reaction into sequencing primers that hybridize 3′ to target residues indicative of unmethylated cytosines. In some embodiments, the second detectably labeled nucleotide is configured to be incorporated in a single-base extension reaction into sequencing primers that hybridize 3′ to target residues indicative of methylated cytosines. In some embodiments, the first detectably labeled nucleotide is configured to be used to generate a first signal that corresponds to the target residues indicative of unmethylated cytosine at the plurality of cytosine residues in the region of interest, and wherein the second detectably labeled nucleotide is configured to be used to generate a second signal that corresponds to the target residues indicative of methylated cytosine at the plurality of cytosine residues in the region of interest. In some embodiments, the kit comprises any suitable number of sequencing primers. In some embodiments, the kit comprises at least 10, 20, 50, 100, or 500 sequencing primers. In some embodiments, the region of interest is any suitable length, such as any described herein. In some embodiments, the region of interest is at least 200 bases, at least 500 bases, at least 1000 bases, or at least 5,000 bases in length. In some embodiments, the kit comprises a reagent for converting the DNA, such as any described herein. In some embodiments, the biological sample is any suitable biological sample, such as a cell or a tissue sample.

In some aspects, provided herein are systems. In some aspects, the systems can be configured to interrogate methylation in a biological sample. In some embodiments, the systems comprise any of the components of the methods and kits provided herein. In some embodiments, the systems comprise means for detecting signals (e.g. an imaging apparatus, such as a microscope) that correspond to and/or are suitable for analyzing methylation status of a region of interest. In some embodiments, the systems comprises means for handling the biological sample, such as a support. In some embodiments, the systems comprise means for analyzing the signals and/or the methylation status of a region of interest, such as computer systems. The computer systems can be programmed to implement any of the methods of the disclosure. In some embodiments, the systems are configured to be used to carry out any of the methods provided herein.

In some aspects, provided herein is a system. In some embodiments, the system comprises a biological sample comprising converted DNA generated by converting DNA. In some embodiments, the nucleotide sequence of the converted DNA is indicative of the methylation state of the DNA. In some embodiments, the DNA comprises a region of interest. In some embodiments, the system comprises a plurality of methylation-state-specific probes. In some embodiments, each methylation-state-specific probe is complementary to a converted DNA target sequence indicative of a methylation state of a target sequence in the region of interest. In some embodiments, one or more of the methylation-state-specific probes is complementary to a converted DNA target sequence indicative of a methylation state of a target sequence in the region of interest. In some embodiments, the plurality of methylation state-specific probes collectively target a plurality of converted DNA target sequences corresponding to a plurality of target sequences in the region of interest. In some embodiments, the plurality of methylation-state-specific probes are configured to be used to generate a methylation-state-specific signal associated with hybridization of methylation-state-specific probes to the converted DNA, for example in accordance with any of the methods provided herein that involve signal detection. In some embodiments, the system further comprises an apparatus for detecting the methylation-state-specific signal in the biological sample. In some embodiments, the methylation-state-specific signal is an optical signal at a location in the biological sample.

In some aspects, provided herein is a system. In some embodiments the system comprises a biological sample comprising converted DNA generated by converting DNA. In some embodiments, the nucleotide sequence of the converted DNA is indicative of the methylation state of the DNA. In some embodiments, the DNA comprises a region of interest. In some embodiments, the system further comprises a plurality of competing probe sets. In some embodiments, each (or one or more) competing probe set comprises: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest. In some embodiments, one or more of the competing probe sets comprises: a first competing probe that is complementary to a first converted DNA target sequence indicative of a first methylation state of a target sequence in the region of interest, and a second competing probe that is complementary to a second converted DNA target sequence indicative of a second methylation state of the target sequence in the region of interest. In some embodiments, the plurality of competing probe sets are configured to be used to generate a first signal associated with hybridization of first competing probes of the plurality of competing probe sets to the converted DNA. In some embodiments, the plurality of competing probe sets are configured to be used to generate a second signal associated with hybridization of second competing probes of the plurality of competing probe sets to the converted DNA. In some embodiments, the system further comprises an apparatus for detecting the first signal and/or second signal (e.g. an imaging system). In some embodiments, the first signal and/or second signal is an optical signal detected at a location in the biological sample. In some embodiments, the optical signal is a fluorescent signal.

In some aspects, provided herein is a system. In some embodiments, the system comprises a biological sample comprising converted DNA generated by converting DNA. In some embodiments, the nucleotide sequence of the converted DNA is indicative of the methylation state of the DNA. In some embodiments, the converted DNA comprises target residues indicative of the methylation state of corresponding cytosine residues in a region of interest of the DNA. In some embodiments, the system comprises a plurality of sequencing primers that hybridize to converted DNA target sequences that are adjacent and 3′ to target residues. In some embodiments, the sequencing primers hybridize to converted DNA target sequences that are immediately 3′ to target residues. In some embodiments, the system comprises a first detectably labeled nucleotide that is configured to be incorporated into sequencing primers that hybridize 3′ to target residues indicative of unmethylated cytosines. In some embodiments, the system comprises a second detectably labeled nucleotide that is configured to be incorporated into sequencing primers that hybridize 3′ to target residues indicative of methylated cytosines. In some embodiments, the first detectably labeled nucleotide is configured to be incorporated in a single-base extension reaction into sequencing primers that hybridize 3′ to target residues indicative of unmethylated cytosines. In some embodiments, the second detectably labeled nucleotide is configured to be incorporated in a single-base extension reaction into sequencing primers that hybridize 3′ to target residues indicative of methylated cytosines. In some embodiments, the first detectably labeled nucleotide is configured to be used to generate a first signal that corresponds to the target residues indicative of unmethylated cytosine at the plurality of cytosine residues in the region of interest, for example in accordance with any of the methods provided herein that involve signal generation or detection. In some embodiments, the second detectably labeled nucleotide is configured to be used to generate a second signal that corresponds to the target residues indicative of methylated cytosine at the plurality of cytosine residues in the region of interest, for example in accordance with any of the methods provided herein that involve signal generation or detection. In some embodiments, the system comprises any suitable number of sequencing primers. In some embodiments, the system comprises at least 10, 20, 50, 100, or 500 sequencing primers. In some embodiments, the system further comprises an apparatus for detecting the first signal and/or second signal. In some embodiments, the first signal and/or second signal is an optical signal (e.g. fluorescent signal) at a location in the biological sample. In some embodiments, the region of interest is any suitable length, such as any provided herein. In some embodiments, the region of interest is at least 200 bases, at least 500 bases, at least 1000 bases, or at least 5,000 bases in length. In some embodiments, the biological sample is any suitable biological sample, such as a cell or a tissue sample.

In some aspects, provided herein are compositions. In some embodiments, the compositions comprise any of the kits or systems provided herein, components thereof, or combinations of components thereof. In some embodiments, the composition comprises any component described in connection with any of the methods provided herein. Such components include but are not limited to any of the biological samples, DNA, converted DNA, regions of interest, reagents for converting DNA, probes (e.g. methylation-state-specific probes), detectable labels, competing probes, competing probe sets, detectable labels, sequencing primers, detectably labeled nucleotides, detectably labeled probes, portions or sub-components of any of the foregoing, or combinations of any of the foregoing. In some embodiments, the composition is generated in the course of performing any of the methods provided herein.

### III. Detection and Analysis

In some aspects, after formation of a hybridization complex comprising nucleic acid probes and/or probe sets, such as any described in Section II, and optionally further processing (e.g., ligation, extension, amplification, or any combination thereof), the method further includes detection of one or more of the probes (e.g., competing probes or sequencing primers hybridized to the target nucleic acid (e.g., converted DNA target sequence) or any products generated therefrom or a derivative thereof. In any of the embodiments herein, the method can further comprise imaging the biological sample to detect a probe or product thereof. In any of the embodiments herein, a sequence of a probe or product thereof, or other generated product can be analyzed in situ in the biological sample. In any of the embodiments herein, the imaging can comprise detecting a signal associated with a detectably labeled probe (e.g., a fluorescently labeled probe) that directly or indirectly binds to a probe or product thereof, such as a competing probe or product thereof. In any of the embodiments herein, the imaging can comprise detecting a signal associated with a detectably labeled nucleotide, such as a detectably labeled nucleotide comprised by a methylation-state-specific probe, competing probe, sequencing primer, or product thereof.

In any of the embodiments herein, a detecting step can comprise contacting the biological sample with one or more detectably-labeled probes that directly or indirectly hybridize to a probe (e.g., a methylation-state-specific probe, competing probe, or sequencing primer) or product thereof.

In any of the embodiments herein, the detecting step can comprise contacting the biological sample with one or more detectable probes that directly hybridize to one or more of the methylation-state-specific probes, competing probes, or sequencing probes. In some instances, the detecting step can comprise contacting the biological sample with one or more detectable probes that indirectly hybridize to one or more of the methylation-state-specific probes, competing probes, or sequencing probes. In any of the embodiments herein, the detecting step can comprise contacting the biological sample with one or more detectable probes that directly or indirectly hybridize to one or more of the methylation-state-specific probes, competing probes, or sequencing probes. In any of the embodiments herein, the detecting step can comprise contacting the biological sample with one or more detectable probes that hybridize directly or indirectly to one or more methylation-state-specific probes, competing probes, sequencing probes, or product thereof. In any of the embodiments herein, the detecting step can comprise a detectable probe that directly or indirectly hybridizes to a product generated using one or more methylation-state-specific probes, competing probes, or sequencing probes, such as an RCA product (e.g., as shown and described for FIG. 7D in Section II.E.).

In some embodiments, the detection may be spatial, e.g., in two or three dimensions. In some embodiments, the detection may be quantitative, e.g., the amount or concentration of a primary nucleic acid probe (e.g., a methylation-state-specific probe, competing probe, or sequencing probe) may be determined. In some embodiments, the primary probes, secondary probes, higher order probes, and/or detectably labeled probes may comprise any of a variety of entities able to hybridize a nucleic acid, e.g., DNA, RNA, LNA, and/or PNA, etc., depending on the application.

In some embodiments, a method disclosed herein may also comprise one or more signal amplification components. In some embodiments, the present disclosure relates to the detection of nucleic acids sequences in situ using probe hybridization and generation of amplified signals associated with hybridized probes (e.g., described in Section II). In some embodiments, a primary probe or product thereof disclosed herein can be detected with a method that comprises signal amplification. In some embodiments, signal amplification may comprise use of one or more of the methylation-state-specific probes, competing probes, or sequencing probes.

Exemplary signal amplification methods include targeted deposition of detectable reactive molecules around the site of probe hybridization, targeted assembly of branched structures (e.g., bDNA or branched assay using locked nucleic acid (LNA)), programmed in situ growth of concatemers by enzymatic rolling circle amplification (RCA) (e.g., as described in US 2019/0055594 incorporated herein by reference), hybridization chain reaction, assembly of topologically catenated DNA structures using serial rounds of chemical ligation (clampFISH), signal amplification via hairpin-mediated concatemerization (e.g., as described in US 2020/0362398 incorporated herein by reference), e.g., primer exchange reactions such as signal amplification by exchange reaction (SABER) or SABER with DNA-Exchange (Exchange-SABER). In some embodiments, a non-enzymatic signal amplification method may be used.

The detectable reactive molecules may comprise tyramide, such as used in tyramide signal amplification (TSA) or multiplexed catalyzed reporter deposition (CARD)-FISH. In some embodiments, the detectable reactive molecule may be releasable and/or cleavable from a detectable label such as a fluorophore. In some embodiments, a method disclosed herein comprises multiplexed analysis of a biological sample comprising consecutive cycles of probe hybridization, fluorescence imaging, and signal removal, where the signal removal comprises removing the fluorophore from a fluorophore-labeled reactive molecule (e.g., tyramide). Exemplary detectable reactive reagents and methods are described in U.S. Pat. No. 6,828,109, US 2019/0376956, WO 2019/236841, US20230384295A1, WO 2020/102094, US20220026433A1, WO 2020/163397, US20220128565A1, WO 2021/067475, and U.S. Pat. No. 12,281,355B2, all of which are incorporated herein by reference in their entireties.

In some embodiments, hybridization chain reaction (HCR) can be used for signal amplification. HCR is an enzyme-free nucleic acid amplification based on a triggered chain of hybridization of nucleic acid molecules starting from HCR monomers, which hybridize to one another to form a nicked nucleic acid polymer. This polymer is the product of the HCR reaction which is ultimately detected in order to indicate the presence of the target analyte. HCR is described in detail in Dirks and Pierce, 2004, PNAS, 101 (43), 15275-15278 and in U.S. Pat. Nos. 7,632,641 and 7,721,721 (see also US 2006/00234261; Chemeris et al, 2008 Doklady Biochemistry and Biophysics, 419, 53-55; Niu et al, 2010, 46, 3089-3091; Choi et al, 2010, Nat. Biotechnol. 28 (11), 1208-1212; and Song et al, 2012, Analyst, 137, 1396-1401). HCR monomers typically comprise a hairpin, or other metastable nucleic acid structure. In the simplest form of HCR, two different types of stable hairpin monomer, referred to here as first and second HCR monomers, undergo a chain reaction of hybridization events to form a long nicked double-stranded DNA molecule when an “initiator” nucleic acid molecule is introduced. The HCR monomers have a hairpin structure comprising a double stranded stem region, a loop region connecting the two strands of the stem region, and a single stranded region at one end of the double stranded stem region. The single stranded region which is exposed (and which is thus available for hybridization to another molecule, e.g. initiator or other HCR monomer) when the monomers are in the hairpin structure may be referred to as the “toehold region” (or “input domain”). The first HCR monomers each further comprise a sequence which is complementary to a sequence in the exposed toehold region of the second HCR monomers. This sequence of complementarity in the first HCR monomers may be referred to as the “interacting region” (or “output domain”). Similarly, the second HCR monomers each comprise an interacting region (output domain), e.g. a sequence which is complementary to the exposed toehold region (input domain) of the first HCR monomers. In the absence of the HCR initiator, these interacting regions are protected by the secondary structure (e.g. they are not exposed), and thus the hairpin monomers are stable or kinetically trapped (also referred to as “metastable”), and remain as monomers (e.g. preventing the system from rapidly equilibrating), because the first and second sets of HCR monomers cannot hybridize to each other. However, once the initiator is introduced, it is able to hybridize to the exposed toehold region of a first HCR monomer, and invade it, causing it to open up. This exposes the interacting region of the first HCR monomer (e.g. the sequence of complementarity to the toehold region of the second HCR monomers), allowing it to hybridize to and invade a second HCR monomer at the toehold region. This hybridization and invasion in turn opens up the second HCR monomer, exposing its interacting region (which is complementary to the toehold region of the first HCR monomers), and allowing it to hybridize to and invade another first HCR monomer. The reaction continues in this manner until all of the HCR monomers are exhausted (e.g. all of the HCR monomers are incorporated into a polymeric chain). Ultimately, this chain reaction leads to the formation of a nicked chain of alternating units of the first and second monomer species. The presence of the HCR initiator is thus required in order to trigger the HCR reaction by hybridization to and invasion of a first HCR monomer. The first and second HCR monomers are designed to hybridize to one another are thus may be defined as cognate to one another. They are also cognate to a given HCR initiator sequence. HCR monomers which interact with one another (hybridize) may be described as a set of HCR monomers or an HCR monomer, or hairpin, system.

An HCR reaction could be carried out with more than two species or types of HCR monomers. For example, a system involving three HCR monomers could be used. In such a system, each first HCR monomer may comprise an interacting region which binds to the toehold region of a second HCR monomer; each second HCR may comprise an interacting region which binds to the toehold region of a third HCR monomer; and each third HCR monomer may comprise an interacting region which binds to the toehold region of a first HCR monomer. The HCR polymerization reaction would then proceed as described above, except that the resulting product would be a polymer having a repeating unit of first, second and third monomers consecutively. Corresponding systems with larger numbers of sets of HCR monomers could readily be conceived. Branching HCR systems have also been devised and described (see, e.g., WO 2020/123742 and US20220064697A1, incorporated herein by reference), and may be used in the methods herein.

In some embodiments, similar to HCR reactions that use hairpin monomers, linear oligo hybridization chain reaction (LO-HCR) can also be used for signal amplification. In some embodiments, provided herein is a method of detecting an analyte in a sample comprising: (i) performing a linear oligo hybridization chain reaction (LO-HCR), wherein an initiator is contacted with a plurality of LO-HCR monomers of at least a first and a second species to generate a polymeric LO-HCR product hybridized to a target nucleic acid molecule, wherein the first species comprises a first hybridization region complementary to the initiator and a second hybridization region complementary to the second species, wherein the first species and the second species are linear, single-stranded nucleic acid molecules; wherein the initiator is provided in one or more parts, and hybridizes directly or indirectly to or is comprised in the target nucleic acid molecule; and (ii) detecting the polymeric product, thereby detecting the analyte. In some embodiments, the first species and/or the second species may not comprise a hairpin structure. In some embodiments, the plurality of LO-HCR monomers may not comprise a metastable secondary structure. In some embodiments, the LO-HCR polymer may not comprise a branched structure. In some embodiments, performing the linear oligo hybridization chain reaction comprises contacting the target nucleic acid molecule with the initiator to provide the initiator hybridized to the target nucleic acid molecule. In any of the embodiments herein, the target nucleic acid molecule and/or the analyte can be an RCA product.

In some embodiments, detection of nucleic acids sequences in situ includes an assembly for branched signal amplification. In some embodiments, the assembly complex comprises an amplifier hybridized directly or indirectly (via one or more oligonucleotides) to a sequence of the probes. In some embodiments, the assembly includes one or more amplifiers each including an amplifier repeating sequence. In some aspects, the one or more amplifiers is labeled. Described herein is a method of using the aforementioned assembly, including for example, using the assembly in multiplexed error-robust fluorescent in situ hybridization (MERFISH) applications, with branched DNA amplification for signal readout. In some embodiments, the amplifier repeating sequence is about 5-30 nucleotides, and is repeated N times in the amplifier. In some embodiments, the amplifier repeating sequence is about 20 nucleotides, and is repeated at least two times in the amplifier. In some aspects, the one or more amplifier repeating sequence is labeled. For exemplary branched signal amplification, see e.g., U.S. Pat. Pub. No. US20200399689A1 and Xia et al., Multiplexed Detection of RNA using MERFISH and branched DNA amplification. Scientific Reports (2019), each of which is fully incorporated by reference herein.

In some embodiments, the probes (e.g., any described in Section II) can be detected with a method that comprises signal amplification by performing a primer exchange reaction (PER). In various embodiments, a primer with a domain on its 3′ end binds to a catalytic hairpin, and is extended with a new domain by a strand displacing polymerase. For example, a primer with domain 1 on its 3′ ends binds to a catalytic hairpin, and is extended with a new domain 1 by a strand displacing polymerase, with repeated cycles generating a concatemer of repeated domain 1 sequences. In various embodiments, the strand displacing polymerase is Bst. In various embodiments, the catalytic hairpin includes a stopper which releases the strand displacing polymerase. In various embodiments, branch migration displaces the extended primer, which can then dissociate. In various embodiments, the primer undergoes repeated cycles to form a concatemer primer. In various embodiments, a plurality of concatemer primers is contacted with a plurality of concatemer primers and a plurality of labeled probes. see e.g., U.S. Pat. Pub. No. US20190106733, which is incorporated herein by reference, for exemplary molecules and PER reaction components.

In some embodiments, the methods comprise determining the sequence of all or a portion of the amplification product, such as one or more barcode sequences present in the amplification product, e.g., by sequencing or by sequential detectable probe hybridization.

In some embodiments, the product or derivative of a first and second probe ligated together after hybridizing to the target nucleic acid can be analyzed by sequencing. In some embodiments, the analysis and/or sequence determination comprises sequencing all or a portion of the amplification product or the probe(s) and/or in situ hybridization to the amplification product or the probe(s). In some embodiments, the sequencing step involves sequencing by hybridization, sequencing by ligation, and/or fluorescent in situ sequencing, hybridization-based in situ sequencing and/or wherein the in situ hybridization comprises sequential fluorescent in situ hybridization. In some embodiments, the analysis and/or sequence determination comprises detecting a polymer generated by a hybridization chain reaction (HCR) reaction, see e.g., US 2017/0009278, which is incorporated herein by reference, for exemplary probes and HCR reaction components. In some embodiments, the detection or determination comprises hybridizing to the amplification product a detection oligonucleotide labeled with a fluorophore, an isotope, a mass tag, or a combination thereof. In some embodiments, the detection or determination comprises imaging the amplification product. In some embodiments, the target nucleic acid is an mRNA in a tissue sample, and the detection or determination is performed when the target nucleic acid and/or the amplification product is in situ in the tissue sample.

In some aspects, the provided methods comprise detecting a probe or product thereof, for example, via binding of a detectable probe and detecting a detectable label comprised by the detectable probe. In some embodiments, the detectable probe comprises a detectable label that can be measured and quantitated. The terms “label” and “detectable label” can refer to a directly or indirectly detectable moiety that is associated with (e.g., conjugated to) a molecule to be detected, e.g., a detectable probe, comprising, but not limited to, fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

The term “fluorophore” can refer to a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. “Autofluorescence” is the general term used to distinguish background fluorescence (that can arise from a variety of sources, including aldehyde fixation, extracellular matrix components, red blood cells, lipofuscin, and the like) from the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

In some embodiments, a detectable probe containing a detectable label can be used to detect one or more polynucleotide(s), probes, and/or amplification products (e.g., amplicon or RCA products) described herein. In some embodiments, the methods involve incubating the detectable probe containing the detectable label with the sample, washing unbound detectable probe, and detecting the label, e.g., by imaging.

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes). Labelling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are described for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or an polynucleotide sequence can be indirectly labeled, for example with a hapten that is then bound by a capture agent, e.g., as disclosed in U.S. Pat. Nos. 5,344,757, 5,702,888, 5,354,657, 5,198,537, 4,849,336, PCT publication WO 91/17160, and U.S. Pat. No. 5,703,562. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detectable probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The “fluorescence microscope” comprises any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detectable probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity-so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also referred to as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PS™, photon scanning tunneling microscopy (PS™), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXS™), and intact tissue expansion microscopy (exM).

In some embodiments, sequencing can be performed in situ. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343 (6177), 1360-1363. In addition, examples of methods and systems for performing in situ sequencing are described in US 2016/0024555, US 2019/0194709, and in U.S. Pat. Nos. 10,138,509, 10,494,662 and 10,179,932. Exemplary techniques for in situ sequencing comprise, but are not limited to, STARmap (described for example in Wang et al., (2018) Science, 361 (6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48 (19): e112, and FISSEQ (described for example in US 2019/0032121). In some cases, sequencing can be performed after the analytes are released from the biological sample.

In some embodiments, sequencing can be performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Exemplary SBS methods comprise those described for example, but not limited to, US 2007/0166705, US 2006/0188901, U.S. Pat. No. 7,057,026, US 2006/0240439, US 2006/0281109, US 2011/005986, US 2005/0100900, U.S. Pat. No. 9,217,178, US 2009/0118128, US 2012/0270305, US 2013/0260372, and US 2013/0079232.

In some embodiments, sequencing can be performed by sequential fluorescence hybridization (e.g., sequencing by hybridization). Sequential fluorescence hybridization can involve sequential hybridization of detection probes comprising an oligonucleotide and a detectable label.

In some embodiments, sequencing can be performed using single molecule sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309:1728-1732, and in U.S. Pat. Nos. 5,599,675; 5,750,341; 6,969,488; 6,172,218; and 6,306,597.

In some embodiments, the primary probes (e.g. methylation-state-specific probes, competing probes, or sequencing probes) or products thereof are targeted by detectably labeled detection oligonucleotides, such as fluorescently labeled oligonucleotides. In some embodiments, one or more decoding schemes are used to decode the signals, such as fluorescence, for sequence determination. In any of the embodiments herein, barcodes (e.g., primary and/or secondary barcode sequences) can be analyzed (e.g., detected or sequenced) using any suitable methods or techniques, comprising those described herein, such as RNA sequential probing of targets (RNA SPOTs), sequential fluorescent in situ hybridization (seqFISH), single-molecule fluorescent in situ hybridization (smFISH), multiplexed error-robust fluorescence in situ hybridization (MERFISH), hybridization-based in situ sequencing (HybISS), in situ sequencing, targeted in situ sequencing, fluorescent in situ sequencing (FISSEQ), or spatially-resolved transcript amplicon readout mapping (STARmap). In some embodiments, the methods provided herein comprise analyzing the barcodes by sequential hybridization and detection with a plurality of labelled probes (e.g., detection oligonucleotides or detectable probes). Exemplary decoding schemes are described in Eng et al., “Transcriptome-scale Super-Resolved Imaging in Tissues by RNA SeqFISH+,” Nature 568 (7751): 235-239 (2019); Chen et al., Science; 348 (6233): aaa6090 (2015); Gyllborg et al., Nucleic Acids Res (2020) 48 (19): e112; U.S. Pat. No. 10,457,980 B2; US 2016/0369329 A1; WO 2018/026873 A1; and US 2017/0220733 A1, all of which are incorporated by reference in their entireties. In some embodiments, these assays enable signal amplification, combinatorial decoding, and error correction schemes at the same time.

In some embodiments, nucleic acid hybridization can be used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), the contents of each of which are herein incorporated by reference in their entireties.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

### IV. Samples, Analytes, and Target Sequences

### A. Samples

A sample disclosed herein can be or be derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein can be any support that is insoluble in aqueous liquid and which allows for positioning of biological samples, analytes, features, and/or reagents (e.g., probes) on the support. In some embodiments, a biological sample can be attached to a substrate. Attachment of the biological sample can be irreversible or reversible, depending upon the nature of the sample and subsequent steps in the analytical method. In certain embodiments, the sample can be attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample can be prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular or padlock probe. In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a padlock probe.

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample can be embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μto about 2 mm.

Additional methods and aspects of hydrogel embedding of biological samples are described for example in Chen et al., Science 347 (6221): 543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample can be contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples can be destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65 (8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) can be isometrically expanded. Isometric expansion methods that can be used include hydration, a preparative step in expansion microscopy, as described in, e.g., Chen et al., Science 347 (6221): 543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl) amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto can be anchored to a polymer matrix. For example, the polymer matrix can be a hydrogel. In some embodiments, one or more of the polynucleotide probe(s) and/or amplification product (e.g., amplicon) thereof can be modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample can be permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample can be permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample can be permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### V. Terminology

Specific terminology is used throughout this disclosure to explain various aspects of the apparatus, systems, methods, and compositions that are described.

Having described some illustrative embodiments of the present disclosure, it should be apparent to those skilled in the art that the foregoing is merely illustrative and not limiting, having been presented by way of example only. Numerous modifications and other illustrative embodiments are within the scope of one of ordinary skill in the art and are contemplated as falling within the scope of the present disclosure. In particular, although many of the examples presented herein involve specific combinations of method acts or system elements, it should be understood that those acts and those elements may be combined in other ways to accomplish the same objectives.

As used herein, the singular forms “a,” “an,” and “the” include plural referents unless the context clearly dictates otherwise. For example, “a” or “an” means “at least one” or “one or more.”

The term “about” as used herein refers to the usual error range for the respective value readily known to the skilled person in this technical field. Reference to “about” a value or parameter herein includes (and describes) embodiments that are directed to that value or parameter per se. In some embodiments, the term “about” refers to a value within 20% of an indicated value. In some embodiments, the term “about” refers to a value within 10% of an indicated value.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### (i) Barcode

A “barcode” is a label, or identifier, that conveys or is capable of conveying information (e.g., information about an analyte in a sample). A barcode can be part of an analyte, or independent of an analyte. A barcode can be attached to an analyte. A barcode can be in a probe or probe set. A particular barcode can be unique relative to other barcodes.

Barcodes can have a variety of different formats. For example, barcodes can include polynucleotide barcodes, random nucleic acid and/or amino acid sequences, and synthetic nucleic acid and/or amino acid sequences. A barcode can be attached to an analyte or to another moiety or structure in a reversible or irreversible manner. A barcode can be associated with an analyte by its inclusion in a probe or probe set that hybridizes to an analyte. A barcode can be added to, for example, a fragment of a deoxyribonucleic acid (DNA) or ribonucleic acid (RNA) sample before or during sequencing of the sample.

In some embodiments, a barcode includes two or more sub-barcodes that together function as a single barcode. For example, a polynucleotide barcode can include two or more polynucleotide sequences (e.g., sub-barcodes) that are separated by one or more non-barcode sequences.

### (ii) Nucleic Acid and Nucleotide

The terms “nucleic acid” and “nucleotide” are intended to be consistent with their use in the art and to include naturally-occurring species or functional analogs thereof. Particularly useful functional analogs of nucleic acids are capable of hybridizing to a nucleic acid in a sequence-specific fashion (e.g., capable of hybridizing to two nucleic acids such that ligation can occur between the two hybridized nucleic acids) or are capable of being used as a template for replication of a particular nucleotide sequence. Naturally-occurring nucleic acids generally have a backbone containing phosphodiester bonds. An analog structure can have an alternate backbone linkage. Naturally-occurring nucleic acids generally have a deoxyribose sugar (e.g., found in deoxyribonucleic acid (DNA)) or a ribose sugar (e.g. found in ribonucleic acid (RNA)).

A nucleic acid can contain nucleotides having any of a variety of suitable analogs of these sugar moieties. A nucleic acid can include native or non-native nucleotides. In this regard, a native deoxyribonucleic acid can have one or more bases selected from the group consisting of adenine (A), thymine (T), cytosine (C), or guanine (G), and a ribonucleic acid can have one or more bases selected from the group consisting of uracil (U), adenine (A), cytosine (C), or guanine (G).

(iii) Probe and Target

A “probe” or a “target,” when used in reference to a nucleic acid or sequence of a nucleic acids, is intended as a semantic identifier for the nucleic acid or sequence in the context of a method or composition, and does not limit the structure or function of the nucleic acid or sequence beyond what is expressly indicated.

### (iv) Oligonucleotide and Polynucleotide

The terms “oligonucleotide” and “polynucleotide” are used interchangeably to refer to a single-stranded multimer of nucleotides from about 2 to about 500 nucleotides in length. Oligonucleotides can be synthetic, made enzymatically (e.g., via polymerization), or using a “split-pool” method. Oligonucleotides can include ribonucleotide monomers (e.g., can be oligoribonucleotides) and/or deoxyribonucleotide monomers (e.g., oligodeoxyribonucleotides). In some examples, oligonucleotides can include a combination of both deoxyribonucleotide monomers and ribonucleotide monomers in the oligonucleotide (e.g., random or ordered combination of deoxyribonucleotide monomers and ribonucleotide monomers). An oligonucleotide can be 4 to 10, 10 to 20, 21 to 30, 31 to 40, 41 to 50, 51 to 60, 61 to 70, 71 to 80, 80 to 100, 100 to 150, 150 to 200, 200 to 250, 250 to 300, 300 to 350, 350 to 400, or 400-500 nucleotides in length, for example. Oligonucleotides can include one or more functional moieties that are attached (e.g., covalently or non-covalently) to the multimer structure. For example, an oligonucleotide can include one or more detectable labels (e.g., a radioisotope or fluorophore).

### (v) Hybridizing, Hybridize, Annealing, and Anneal

The terms “hybridizing,” “hybridize,” “annealing,” and “anneal” are used interchangeably in this disclosure, and refer to the pairing of substantially complementary or complementary nucleic acid sequences within two different molecules. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

### (vi) Primer

A “primer” is a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence.

(vii) Antibody

An “antibody” is a polypeptide molecule that recognizes and binds to a complementary target antigen. As used herein, the term antibody refers to an antibody molecule of any class, or any sub-fragment thereof, such as a Fab. Antibodies typically have a molecular structure shape that resembles a Y shape. Naturally-occurring antibodies, referred to as immunoglobulins, belong to one of the immunoglobulin classes IgG, IgM, IgA, IgD, and IgE. Antibodies can also be produced synthetically. For example, recombinant antibodies, which are monoclonal antibodies, can be synthesized using synthetic genes by recovering the antibody genes from source cells, amplifying into an appropriate vector, and introducing the vector into a host to cause the host to express the recombinant antibody. In general, recombinant antibodies can be cloned from any species of antibody-producing animal using suitable oligonucleotide primers and/or hybridization probes. Recombinant techniques can be used to generate antibodies and antibody fragments, including non-endogenous species.

Synthetic antibodies can be derived from non-immunoglobulin sources. For example, antibodies can be generated from nucleic acids (e.g., aptamers), and from non-immunoglobulin protein scaffolds (such as peptide aptamers) into which hypervariable loops are inserted to form antigen binding sites. Synthetic antibodies based on nucleic acids or peptide structures can be smaller than immunoglobulin-derived antibodies, leading to greater tissue penetration.

Antibodies can also include affimer proteins, which are affinity reagents that typically have a molecular weight of about 12-14 kDa. Affimer proteins generally bind to a target (e.g., a target protein) with both high affinity and specificity. Examples of such targets include, but are not limited to, ubiquitin chains, immunoglobulins, and C-reactive protein. In some embodiments, affimer proteins are derived from cysteine protease inhibitors, and include peptide loops and a variable N-terminal sequence that provides the binding site.

Antibodies can also refer to an “epitope binding fragment” or “antibody fragment,” which as used herein, generally refers to a portion of a complete antibody capable of binding the same epitope as the complete antibody, albeit not necessarily to the same extent. Although multiple types of epitope binding fragments are possible, an epitope binding fragment typically comprises at least one pair of heavy and light chain variable regions (VH and VL, respectively) held together (e.g., by disulfide bonds) to preserve the antigen binding site, and does not contain all or a portion of the Fc region. Epitope binding fragments of an antibody can be obtained from a given antibody by any suitable technique (e.g., recombinant DNA technology or enzymatic or chemical cleavage of a complete antibody), and typically can be screened for specificity in the same manner in which complete antibodies are screened. In some embodiments, an epitope binding fragment comprises an F(ab′) 2 fragment, Fab′ fragment, Fab fragment, Fd fragment, or Fv fragment. In some embodiments, the term “antibody” includes antibody-derived polypeptides, such as single chain variable fragments (scFv), diabodies or other multimeric scFvs, heavy chain antibodies, single domain antibodies, or other polypeptides comprising a sufficient portion of an antibody (e.g., one or more complementarity determining regions (CDRs)) to confer specific antigen binding ability to the polypeptide.

(viii) Label, Detectable Label, and Optical Label

The terms “detectable label,” “optical label,” and “label” are used interchangeably herein to refer to a directly or indirectly detectable moiety that is associated with (e.g., conjugated to) a molecule to be detected, e.g., a probe for in situ assay, or analyte. The detectable label can be directly detectable by itself (e.g., radioisotope labels or fluorescent labels) or, in the case of an enzymatic label, can be indirectly detectable, e.g., by catalyzing chemical alterations of a substrate compound or composition, which substrate compound or composition is directly detectable. Detectable labels can be suitable for small scale detection and/or suitable for high-throughput screening. As such, suitable detectable labels include, but are not limited to, radioisotopes, fluorophores, chemiluminescent compounds, bioluminescent compounds, and dyes.

The detectable label can be qualitatively detected (e.g., optically or spectrally), or it can be quantified. Qualitative detection generally includes a detection method in which the existence or presence of the detectable label is confirmed, whereas quantifiable detection generally includes a detection method having a quantifiable (e.g., numerically reportable) value such as an intensity, duration, polarization, and/or other properties. For example, detectably labelled features can include a fluorescent, a colorimetric, or a chemiluminescent label attached to a bead (see, for example, Rajeswari et al., J. Microbiol Methods 139:22-28, 2017, and Forcucci et al., J. Biomed Opt. 10:105010, 2015, the entire contents of each of which are incorporated herein by reference).

In some embodiments, a plurality of detectable labels can be attached to a polynucleotide disclosed herein (e.g., a probe, probe set, or decoy oligonucleotide). For example, detectable labels can be incorporated during nucleic acid polymerization or amplification (e.g., Cy5®-labelled nucleotides, such as Cy5®-dCTP). Any suitable detectable label can be used. In some embodiments, the detectable label is a fluorophore. For example, the fluorophore can be from a group that includes: 7-AAD (7-Aminoactinomycin D), Acridine Orange (+DNA), Acridine Orange (+RNA), Alexa Fluor® 350, Alexa Fluor® 430, Alexa Fluor® 488, Alexa Fluor® 532, Alexa Fluor® 546, Alexa Fluor® 555, Alexa Fluor® 568, Alexa Fluor® 594, Alexa Fluor® 633, Alexa Fluor® 647, Alexa Fluor® 660, Alexa Fluor® 680, Alexa Fluor® 700, Alexa Fluor® 750, Allophycocyanin (APC), AMCA/AMCA-X, 7-Aminoactinomycin D (7-AAD), 7-Amino-4-methylcoumarin, 6-Aminoquinoline, Aniline Blue, ANS, APC-Cy7, ATTO-TAG™ CBQCA, ATTO-TAG™ FQ, Auramine O-Feulgen, BCECF (high pH), BFP (Blue Fluorescent Protein), BFP/GFP FRET, BOBO™-1/BO-PRO™-1, BOBO™-3/BO-PRO™-3, BODIPY® FL, BODIPY® TMR, BODIPY® TR-X, BODIPY® 530/550, BODIPY® 558/568, BODIPY® 564/570, BODIPY® 581/591, BODIPY® 630/650-X, BODIPY® 650-665-X, BTC, Calcein, Calcein Blue, Calcium Crimson™, Calcium Green-1™, Calcium Orange™, Calcofluor® White, 5-Carboxyfluoroscein (5-FAM), 5-Carboxynaphthofluoroscein, 6-Carboxyrhodamine 6G, 5-Carboxytetramethylrhodamine (5-TAMRA), Carboxy-X-rhodamine (5-ROX), Cascade Blue®, Cascade Yellow™, CCF2 (GeneBLAzer™), CFP (Cyan Fluorescent Protein), CFP/YFP FRET, Chromomycin A3, Cl-NERF (low pH), CPM, 6-CR 6G, CTC Formazan, Cy2®, Cy3®, Cy3.5®, Cy5®, Cy5.5®, Cy7®, Cychrome (PE-Cy5), Dansylamine, Dansyl cadaverine, Dansylchloride, DAPI, Dapoxyl, DCFH, DHR, DiA (4-Di-16-ASP), DiD (DilC18 (5)), DIDS, Dil (DilC18 (3)), DiO (DiOC18 (3)), DiR (DilC18 (7)), Di-4 ANEPPS, Di-8 ANEPPS, DM-NERF (4.5-6.5 pH), DsRed (Red Fluorescent Protein), EBFP, ECFP, EGFP, ELF®-97 alcohol, Eosin, Erythrosin, Ethidium bromide, Ethidium homodimer-1 (EthD-1), Europium (III) Chloride, 5-FAM (5-Carboxyfluorescein), Fast Blue, Fluorescein-dT phosphoramidite, FITC, Fluo-3, Fluo-4, FluorX®, Fluoro-Gold™ (high pH), Fluoro-Gold™ (low pH), Fluoro-Jade, FM® 1-43, Fura-2 (high calcium), Fura-2/BCECF, Fura Red™ (high calcium), Fura Red™/Fluo-3, GeneBLAzer™ (CCF2), GFP Red Shifted (rsGFP), GFP Wild Type, GFP/BFP FRET, GFP/DsRed FRET, Hoechst 33342 & 33258, 7-Hydroxy-4-methylcoumarin (pH 9), 1,5 IAEDANS, Indo-1 (high calcium), Indo-1 (low calcium), Indodicarbocyanine, Indotricarbocyanine, JC-1, 6-JOE, JOJO™-1/JO-PRO™-1, LDS 751 (+DNA), LDS 751 (+RNA), LOLO™-1/LO-PRO™-1, Lucifer Yellow, LysoSensor™ Blue (pH 5), LysoSensor™ Green (pH 5), LysoSensor™ Yellow/Blue (pH 4.2), LysoTracker® Green, LysoTracker® Red, LysoTracker® Yellow, Mag-Fura-2, Mag-Indo-1, Magnesium Green™, Marina Blue®, 4-Methylumbelliferone, Mithramycin, MitoTracker® Green, MitoTracker® Orange, MitoTracker® Red, NBD (amine), Nile Red, Oregon Green® 488, Oregon Green® 500, Oregon Green® 514, Pacific Blue, PBF1, PE (R-phycoerythrin), PE-Cy5, PE-Cy7, PE-Texas Red, PerCP (Peridinin chlorphyll protein), PerCP-Cy5.5 (TruRed), PharRed (APC-Cy7), C-phycocyanin, R-phycocyanin, R-phycoerythrin (PE), PI (Propidium Iodide), PKH26, PKH67, POPO™-1/PO-PRO™-1, POPO™-3/PO-PRO™-3, Propidium Iodide (PI), PyMPO, Pyrene, Pyronin Y, Quantam Red (PE-Cy5), Quinacrine Mustard, R670 (PE-Cy5), Red 613 (PE-Texas Red), Red Fluorescent Protein (DsRed), Resorufin, RH 414, Rhod-2, Rhodamine B, Rhodamine Green™, Rhodamine Red™, Rhodamine Phalloidin, Rhodamine 110, Rhodamine 123, 5-ROX (carboxy-X-rhodamine), S65A, S65C, S65L, S65T, SBFI, SITS, SNAFL®-1 (high pH), SNAFL®-2, SNARF®-1 (high pH), SNARF®-1 (low pH), Sodium Green™, SpectrumAqua®, SpectrumGreen® #1, SpectrumGreen® #2, SpectrumOrange®, SpectrumRed®, SYTO® 11, SYTO® 13, SYTO® 17, SYTO® 45, SYTOX® Blue, SYTOX® Green, SYTOX® Orange, 5-TAMRA (5-Carboxytetramethylrhodamine), Tetramethylrhodamine (TRITC), Texas Red®/Texas Red®-X, Texas Red®-X (NHS Ester), Thiadicarbocyanine, Thiazole Orange, TOTO®-1/TO-PRO®-1, TOTO®-3/TO-PRO®-3, TO-PRO®-5, Tri-color (PE-Cy5), TRITC (Tetramethylrhodamine), TruRed (PerCP-Cy5.5), WW 781, X-Rhodamine (XRITC), Y66F, Y66H, Y66W, YFP (Yellow Fluorescent Protein), YOYO®-1/YO-PRO®-1, YOYO®-3/YO-PRO®-3, 6-FAM (Fluorescein), 6-FAM (NHS Ester), 6-FAM (Azide), HEX, TAMRA (NHS Ester), Yakima Yellow, MAX, TET, TEX615, ATTO 488, ATTO 532, ATTO 550, ATTO 565, ATTO Rho101, ATTO 590, ATTO 633, ATTO 647N, TYE 563, TYE 665, TYE 705, 5′ IRDye® 700, 5′ IRDye® 800, 5′ IRDye® 800CW (NHS Ester), WellRED D4 Dye, WellRED D3 Dye, WellRED D2 Dye, Lightcycler® 640 (NHS Ester), and Dy 750 (NHS Ester).

As mentioned above, in some embodiments, a detectable label is or includes a luminescent or chemiluminescent moiety. Common luminescent/chemiluminescent moieties include, but are not limited to, peroxidases such as horseradish peroxidase (HRP), soybean peroxidase (SP), alkaline phosphatase, and luciferase. These protein moieties can catalyze chemiluminescent reactions given the appropriate substrates (e.g., an oxidizing reagent plus a chemiluminescent compound. A number of compound families provide chemiluminescence under a variety of conditions. Non-limiting examples of chemiluminescent compound families include 2,3-dihydro-1,4-phthalazinedione luminol, 5-amino-6,7,8-trimethoxy- and the dimethylamino[ca]benz analog. These compounds can luminesce in the presence of alkaline hydrogen peroxide or calcium hypochlorite and base. Other examples of chemiluminescent compound families include, e.g., 2,4,5-triphenylimidazoles, para-dimethylamino and -methoxy substituents, oxalates such as oxalyl active esters, p-nitrophenyl, N-alkyl acridinum esters, luciferins, lucigenins, or acridinium esters. In some embodiments, a detectable label is or includes a metal-based or mass-based label. For example, small cluster metal ions, metals, or semiconductors may act as a mass code. In some examples, the metals can be selected from Groups 3-15 of the periodic table, e.g., Y, La, Ag, Au, Pt, Ni, Pd, Rh, Ir, Co, Cu, Bi, or a combination thereof.

As used herein, the term “fluorescent label” comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

### EXAMPLES

The following examples are included for illustrative purposes only and are not intended to limit the scope of the present disclosure.

### Example 1: Analysis of Methylation Status of a Region of Interest Using Methylation-State-Specific Probes

This example demonstrates a method of using methylation-state-specific probes to interrogate and analyze the methylation status of a region of interest in DNA in a biological sample.

The DNA in the biological sample is converted to generate converted DNA. In some embodiments, the DNA conversion comprises performing bisulfite conversion, or enzymatic conversion, as described herein. In this example, following DNA conversion, unmethylated cytosines in the DNA are converted to uracil, whereas methylated cytosines (methylcytosine and/or hydroxymethylcytosine) are not converted to uracil. Thus, the sequence of the converted DNA corresponds to and is indicative of the methylation state of the DNA prior to and/or after conversion.

Next, the biological sample is contacted with first methylation-state-specific probes that are complementary to converted DNA target sequences indicative of first methylation states of corresponding target sequences in the region of interest in the DNA. The first methylation-state-specific probes in this example hybridize to converted DNA target sequences indicative of target sequences in which all of the CpG cytosines are methylated (e.g. as shown in FIG. 1). Following hybridization, unhybridized probes are washed out of the sample. The first methylation-state-specific probes are associated with a first detectable label, which in this example is a fluorophore. A first signal is generated from the first detectable label, and the first signal is used to analyze the methylation status of the region of interest. A high-intensity first signal is indicative of a high proportion of CpG cytosines being methylated in the region of interest (e.g. as shown in FIG. 1; left), whereas a low-intensity first signal is indicative of a low proportion of CpG cytosines being methylated in the region of interest (e.g. as shown in FIG. 1; right). The first signal can be analyzed in comparison to any suitable reference signal. The first signal can also be analyzed in comparison to a second signal generated from a second detectable label associated with second methylation-state-specific probes that hybridize to converted DNA target sequences indicative of target sequences having a second methylation state (e.g. target sequences in which none of the CpG cytosines are methylated). Analysis of the methylation status of the region of interest can comprise analysis and/or comparison of the first signal and/or second signal. Third and/or further (e.g. fourth, fifth, sixth, etc.) signals corresponding to third and/or further methylation states can also be generated and analyzed.

Any of the signals can be detected at one or more specific locations in the biological sample, such as at a spatially localized position of the region of interest in the biological sample. The spatially localized position of the region of interest can be determined by any suitable means, and may be determined using any of the methylation-state-specific probes themselves or using a separate set of probes.

### Example 2: Analysis of the Methylation Status of a Region of Interest Using Competing Probe Sets

This example demonstrates a method of using competing probe sets to interrogate and analyze the methylation status of a region of interest in DNA in a biological sample.

The DNA in the biological sample is converted to generated converted DNA. In some embodiments, the DNA conversion comprises performing bisulfite conversion, or enzymatic conversion, as described herein. In this example, following DNA conversion, unmethylated cytosines in the DNA are converted to uracil, whereas methylated cytosines (methylcytosine and/or hydroxymethylcytosine) are not converted to uracil. Thus, the sequence of the converted DNA corresponds to and is indicative of the methylation state of the DNA prior to and/or after conversion.

Next, the biological sample is contacted with a plurality of competing probe sets for analyzing the methylation status of the region of interest, for example as shown in FIGS. 2A-2B. Each competing probe set corresponds to (e.g., is used for interrogating the methylation state of) a different target sequence in the DNA. Each competing probe of a single competing probe set is complementary to a different converted DNA target sequence that is indicative of a different methylation state of a single target sequence. For example, for a given competing probe set for a given target sequence, a first competing probe of the competing probe set is complementary to a first converted DNA target sequence indicative of a first methylation state of the target sequence (e.g. a 0% methylation state, such as a methylation state in which all CpG cytosines in the target sequence are unmethylated), and a second competing probe of the competing probe set is complementary to a second converted DNA target sequence indicative of a second methylation state of the target sequence (e.g. a 100% methylation state, such as a methylation state in which all CpG cytosines in the target sequence are methylated). In this example, all first probes of the competing probe sets correspond to 0% methylation states, and are associated with a first detectable label which is also associated with 0% methylation states; and all second probes of the competing probes correspond to 100% methylation states, and are associated with a second detectable label which is also associated with 100% methylation states. For example, first competing probes corresponding to 0% methylation states are labeled with GFP, and second competing probes corresponding to 100% methylation states are labeled with Cy5. The plurality of competing probe sets are added to the sample and hybridized under stringent conditions such that the perfectly complementary competing probes preferentially hybridize to the converted DNA target sequences corresponding to specific methylation states. Following hybridization of competing probes, unhybridized competing probes are washed out of the sample.

Next, a first signal can be collectively generated from the first detectable labels of all hybridized first competing probes, and the first signal can be used to analyze the methylation status of the region of interest. A second signal can also be collectively generated from the second detectable labels of all hybridized second competing probes, and the second signal can also be used to analyze the methylation status of the region of interest.

In this example, a high-intensity first signal is indicative of a small proportion of CpG cytosines being methylated in the region of interest (e.g. as shown in FIG. 2B; top), whereas a low-intensity first signal is indicative of a large proportion of CpG cytosines being methylated (e.g. as shown in FIG. 2B; bottom). Similarly, a high-intensity second signal is indicative of a large proportion of CpG cytosines being methylated in the region of interest (e.g. as shown in FIG. 2B; top), whereas a low-intensity second signal is indicative of a small proportion of CpG cytosines being methylated (e.g. as shown in FIG. 2B; bottom). The first signal and/or second signal can be analyzed alone or in comparison to any suitable reference signal. The first signal and second signal can be analyzed in comparison to one another.

Any of the signals can be detected at one or more specific locations in the biological sample, such as at a spatially localized position of the region of interest in the biological sample. The spatially localized position of the region of interest can be determined by any suitable means, and may be determined using any of the competing probes themselves or using a separate set of probes.

### Example 3: Analysis of the Methylation Status of a Region of Interest Using Sequencing Primers

This example demonstrates a method of using sequencing primers to interrogate and analyze the methylation status of a region of interest in DNA in a biological sample.

The DNA in the biological sample is converted to generated converted DNA. In some embodiments, the DNA conversion comprises performing bisulfite conversion, or enzymatic conversion, as described herein. In this example, following DNA conversion, unmethylated cytosines in the DNA are converted to uracil, whereas methylated cytosines (methylcytosine and/or hydroxymethylcytosine) are not converted to uracil. Thus, the sequence of the converted DNA corresponds to and is indicative of the methylation state of the DNA prior to and/or after conversion.

Next, the biological sample is contacted with a plurality of sequencing primers for interrogating the region of interest, for example as shown in FIGS. 4A-4B. In this example, the sequencing primers hybridize to converted DNA target sequences that are adjacent and immediately 3′ to target residues in the converted DNA that are indicative of the methylation state of corresponding cytosine residues in the region of interest. In this example, uracil target residues are indicative of unmethylated cytosine residues in the region of interest, and cytosine target residues are indicative of methylated cytosine residues in the region of interest.

Next, a single-base extension reaction is performed to incorporate first detectably labeled nucleotides or second detectably labeled nucleotides into the sequencing primers. The single-base extension reaction a) incorporates a first detectably labeled nucleotide into sequencing primers that hybridize 3′ to target residues indicative of unmethylated cytosines; and b) incorporates a second detectably labeled nucleotide into sequencing primers that hybridize 3′ to target residues indicative of methylated cytosines. In this example, the first detectably labeled nucleotide can be adenine, which is complementary to a uracil target residue indicative of unmethylated cytosine (e.g. as shown in FIG. 5; left). The second detectably labeled nucleotide can be guanine, which is complementary to a cytosine target residue indicative of methylated cytosine (e.g. as shown in FIG. 5; right).

Following the single-base extension reaction, a first signal is generated collectively from the incorporated first detectably labeled nucleotides, and a second signal is generated collectively from the incorporated second detectably labeled nucleotides. In some embodiments, only a first or second signal is generated. In some embodiments, both a first and second signal is generated. The first signal and/or second signal can be used to analyze the methylation status of the region of interest. In this example, a high-intensity first signal is indicative of a small proportion of CpG cytosines being methylated in the region of interest (e.g. as shown in FIG. 4B; top), whereas a low-intensity first signal is indicative of a large proportion of CpG cytosines being methylated (e.g. as shown in FIG. 4B; bottom). Similarly, a high-intensity second signal is indicative of a large proportion of CpG cytosines being methylated in the region of interest (e.g. as shown in FIG. 4B; top), whereas a low-intensity second signal is indicative of a small proportion of CpG cytosines being methylated (e.g. as shown in FIG. 4B; bottom). The first signal and/or second signal can be analyzed alone or in comparison to any suitable reference signal. The first signal and second signal can be analyzed in comparison to one another.

Any of the signals can be detected at one or more specific locations in the biological sample, such as at a spatially localized position of the region of interest in the biological sample. The spatially localized position of the region of interest can be determined by any suitable means, and may be determined using the sequencing probes themselves or using a separate set of probes.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1. A method for interrogating methylation of a region of interest in a deoxyribonucleic acid (DNA) comprising:
providing a biological sample comprising converted DNA generated by converting the DNA, wherein the nucleotide sequence of the converted DNA is indicative of the methylation state of the DNA;
contacting the biological sample with a plurality of methylation-state-specific probes,
wherein each methylation-state-specific probe is complementary to a converted DNA target sequence indicative of a methylation state of a target sequence in the region of interest, and
wherein the plurality of methylation state-specific probes collectively target a plurality of converted DNA target sequences corresponding to a plurality of target sequences in the region of interest; and
detecting a methylation-state-specific signal associated with hybridization of one or more of the methylation-state-specific probes to the converted DNA.

2. (canceled)

3. The method of claim 1, wherein the methylation-state-specific signal is collectively generated from the methylation-state-specific probes that hybridize to the converted DNA.

4. The method of claim 1, wherein:
the plurality of methylation-state-specific probes is a plurality of first methylation-state-specific probes;
each first methylation-state-specific probe is complementary to a converted DNA target sequence indicative of a first methylation state of a target sequence in the region of interest;
the methylation-state-specific signal is a first methylation-state-specific signal; and
the method comprises detecting the first methylation-state-specific signal associated with hybridization of first methylation-state-specific probes to the converted DNA.

5. The method of claim 4, wherein the method further comprises:
contacting the biological sample with a plurality of second methylation-state-specific probes,
wherein each second methylation-state-specific probe is complementary to a converted DNA target sequence indicative of a second methylation state of a target sequence in the region of interest,
wherein the plurality of second methylation state-specific probes collectively target a plurality of converted DNA target sequences corresponding to a plurality of target sequences in the region of interest; and
detecting a second methylation-state-specific signal associated with hybridization of one or more of the second methylation-state-specific probes to the converted DNA.

6. The method of claim 5, wherein the first methylation-state-specific signal is collectively generated from the first methylation-state-specific probes that hybridize to the converted DNA; and/or
wherein the second methylation-state-specific signal is collectively generated from the second methylation-state-specific probes that hybridize to the converted DNA.

7. The method of claim 5, wherein the plurality of converted DNA target sequences targeted by the first methylation state-specific probes and the plurality of converted DNA target sequences targeted by the second methylation state-specific probes correspond to the same plurality of target sequences in the region of interest.

8. (canceled)

9. (canceled)

10. The method of claim 1, wherein:
the plurality of methylation-state-specific probes comprises at least 3, 5, 10, 20, 50, 100, or 500 methylation-state-specific probes.

11. (canceled)

12. (canceled)

13. The method of claim 5, wherein the method comprises measuring the size, intensity, and/or abundance of the first methylation-state-specific signal, the second methylation-state-specific signal, and/or a reference signal.

14. The method of claim 13, wherein the method comprises comparing the first methylation-state-specific signal, and/or the second methylation-state-specific signal to the reference signal, and/or wherein the method comprises comparing the first methylation-state-specific signal to the second methylation-state-specific signal.

15-22. (canceled)

23. The method of claim 1, wherein:
the methylation-state-specific probes are directly or indirectly associated with a detectable label, and detecting the methylation-state-specific signal comprises detecting the detectable label.

24-30. (canceled)

31. A method for interrogating methylation of a region of interest in a deoxyribonucleic acid (DNA) comprising:
providing a biological sample comprising converted DNA generated by converting the DNA, wherein the nucleotide sequence of the converted DNA is indicative of the methylation state of the DNA,
contacting the biological sample with a plurality of competing probe sets, each competing probe set comprising:
a first competing probe that is complementary to a first converted DNA target sequence indicative of a first methylation state of a target sequence in the region of interest, and
a second competing probe that is complementary to a second converted DNA target sequence indicative of a second methylation state of the target sequence in the region of interest; and
detecting a first signal associated with hybridization of first competing probes of the plurality of competing probe sets to the converted DNA.

32. The method of claim 31, wherein the method further comprises detecting a second signal associated with hybridization of second competing probes of the plurality of competing probe sets to the converted DNA.

33-37. (canceled)

38. The method of claim 32, wherein:
the first signal corresponds to the first methylation states of the target sequences; and
the second signal corresponds to the second methylation states of the target sequences.

39-41. (canceled)

42. The method of claim 31, wherein the plurality of competing probe sets comprises at least 3, 5, 10, 20, 50, 100, or 500 competing probe sets for at least 3, 5, 10, 20, 50, 100, or 500 target sequences in the region of interest, respectively.

43-46. (canceled)

47. The method of claim 32, wherein the method comprises measuring the size, intensity, and/or abundance of the first signal, second signal, and/or a reference signal.

48. The method of claim 47, wherein the method comprises comparing at least two of: the first signal, second signal, and reference signal.

49. (canceled)

50. The method of claim 38, wherein increasing size, intensity, and/or abundance of a detected signal is indicative of a methylation status of the region of interest that is increasingly similar to the methylation state to which the signal corresponds.

51-58. (canceled)

59. The method of claim 32, wherein:
the first competing probes of the plurality of competing probe sets are directly or indirectly associated with a first detectable label corresponding to the first methylation state, and detecting the first signal comprises detecting the first detectable label; and
the second competing probes of the plurality of competing probe sets are directly or indirectly associated with a second detectable label corresponding to the second methylation state, and detecting the second signal comprises detecting the second detectable label.

60-66. (canceled)

67. The method of claim 32, wherein:
the first signal is collectively generated from the first competing probes of the plurality of competing probe sets that hybridize to the converted DNA; and
the second signal is collectively generated from the second competing probes of the plurality of competing probe sets that hybridize to the converted DNA.

68-71. (canceled)

72. A method for interrogating methylation of a region of interest in a deoxyribonucleic acid (DNA) comprising:
providing a biological sample comprising converted DNA generated by converting the DNA, wherein the nucleotide sequence of the converted DNA is indicative of the methylation state of the DNA, and wherein the converted DNA comprises target residues indicative of the methylation state of corresponding cytosine residues in the region of interest,
contacting the biological sample with a plurality of sequencing primers that hybridize to converted DNA target sequences that are adjacent and 3′ to target residues;
performing an extension reaction that a) incorporates a first detectably labeled nucleotide into sequencing primers that hybridize 3′ to target residues indicative of unmethylated cytosines and/or b) incorporates a second detectably labeled nucleotide into sequencing primers that hybridize 3′ to target residues indicative of methylated cytosines; and
detecting a first signal associated with incorporation of the first detectably labeled nucleotide and/or detecting a second signal associated with the second detectably labeled nucleotide.

73-208. (canceled)
